(19) United States
(12) Patent Application Publication
(10) Pub. No.: US 2025/0171533 A1
(43) Pub. Date: May 29, 2025

(54) KIR3DL3 INHIBITORS AND IMMUNE CELL ACTIVATING AGENTS

(71) Applicant: NextPoint Therapeutics, Inc., Cambridge, MA (US)

(72) Inventors: Scott Chappel, Milton, MA (US); Bijan Etemad-Gilbertson, Jamaica Plain, MA (US); Detlev Biniszkiewicz, Boston, MA (US)

(21) Appl. No.: 18/839,953

(22) PCT Filed: Feb. 21, 2023
(86) PCT No.: PCT/US23/13537

§ 371 Date: Aug. 20, 2024

Related U.S. Application Data
(60) Provisional application No. 63/418,105, filed on Oct. 21, 2022.
(60) Provisional application No. 63/312,507, filed on Feb. 22, 2022.

(51) Int. Cl. C07K 16/28; A61K 39/00; A61P 35/00

(52) U.S. Cl. CPC C07K 16/2803; A61P 35/00; A61K 2039/505; C07K 2317/24; C07K 2317/92

(57) ABSTRACT

The present disclosure pertains to KIR3DL3 inhibitors (e.g., anti-KIR3DL3 antibodies or antigen-binding fragments thereof) and immune cell activating agents and uses thereof, such as to modify immune effector cells.

### CROSS REFERENCE TO RELATED APPLICATIONS

This application claims priority to U.S. Provisional Patent Application No. 63/312,507, filed on Feb. 22, 2022, and U.S. Provisional Patent Application No. 63/418,105, filed on Oct. 21, 2022, the contents of each of which are incorporated herein by reference in their entirety.

### BACKGROUND

Although immunotherapies have been investigated for many diseases and disorders, including cancer, functional limitations have been encountered that still need to be addressed. The immune system is tightly controlled by a network of costimulatory and co-inhibitory ligands and receptors. Immune checkpoints negatively regulate immune response progression based on complex interactions. Currently available immune checkpoint inhibitors can modulate immune responses in some patients, but immune checkpoint expression and interactions with natural binding partners can vary between patients.

Therefore, a need exists for the development of new therapeutic modalities optimized to target immune checkpoint pathways.

### SUMMARY OF THE INVENTION

Killer-cell immunoglobulin-like receptor (KIR) proteins include either two (KIR2D) or three (KIR3D) immunoglobulin-like extracellular domains. KIR3DL3 is a member of the KIR family and a receptor demonstrated to be found on both T cells and NK cells. HHLA2, a B7 gene family member, is broadly expressed in a variety of tumors and antigen presenting cells. HHLA2 binding to KIR3DL3 has been shown to inhibit the immune response of activated T cells.

The present disclosure encompasses, inter alia, the discovery of anti-KIR3DL3 antibodies and antigen-binding fragments thereof described herein. Importantly, anti-KIR3DL3 antibodies and antigen-binding fragments thereof described herein can have one or more of the following properties: (1) specifically bind KIR3DL3 (e.g., human KIR3DL3) with high affinity, (2) specifically bind KIR3DL3 expressed on NK cells and/or T cells, (3) block binding of KIR3DL3 to HHLA2, (4) block HHLA2-mediated suppressive activity in T cells, (5) enhance NK cell killing of HHLA2-expressing tumor cells, and (6) enhance anti-tumor activity. Accordingly, the present disclosure provides anti-KIR3DL3 antibodies and antigen-binding fragments thereof described herein that are useful for methods of treating diseases, disorders, and conditions, such as cancers described herein, and for methods of modulating an immune response.

While inhibitors of KIR3DL3 have been investigated as immunotherapies, the present disclosure encompasses, inter alia, the recognition that immune cell activating agents described herein can (i) increase KIR3DL3 expression in order to enhance functional KIR3DL3 inhibition and/or (ii) increases (e.g., epigenetically) the strength of a KIR3DL3 promoter. Accordingly, the present disclosure provides several examples of such immune cell activating agents that are particularly useful in combination with KIR3DL3 inhibitors for treating a variety of cancers, including solid tumors, such as renal cell carcinoma (RCC).

In one aspect, the disclosure provides methods of treating a subject having a disease, disorder or condition comprising administering a population of modified immune effector cells, wherein, prior to administering, a population of immune effector cells were contacted with at least one immune cell activating agent and at least one KIR3DL3 inhibitor, thereby forming a population of modified immune effector cells.

In another aspect, the disclosure provides methods of treating a subject having a disease, disorder, or condition comprising: (i) administering a population of modified immune effector cells to the subject, wherein a population of immune effector cells were contacted with at least one immune cell activating agent prior to administering, thereby forming a population of modified immune effector cells, and (ii) administering at least one KIR3DL3 inhibitor to the subject.

In some embodiments, at least one KIR3DL3 inhibitor is or comprises an anti-KIR3DL3 antibody or an antigen-binding fragment thereof, a miRNA, a shRNA, a siRNA, a CRISPR/Cas guide system, a TALEN, a ZFN, and/or a demethylating agent. In some embodiments, an antigen-binding fragment comprises an scFv, Fab, Fab′, F(ab′)2, Fc, or nanobody.

In some embodiments, a demethylating agent comprises or is 5-aza-2-deoxycytidine (Aza), 5-azacytidine, 1-β-D-arabinofuranosil-5-azacytosine, or dihydro-5-azacytidine.

In some embodiments, an immune cell activating agent results in T cell proliferation and/or increased endogenous expression of at least one cytokine.

In some embodiments, an immune cell activating agent comprises or is a cytokine agent. In some embodiments, a cytokine agent is or comprises IL-2, IL-15, IL-12, IL-17, IL-18, IL-21, IFNγ, and/or TNFα. In some embodiments, IL-2 binds to IL-2Rα, IL-2Rβ, or IL-2Rγ. In some embodiments, IL-2 expands only T cells and does not substantially expand Tregs. In some embodiments, a cytokine agent is or comprises an inhibitor of a suppressor of cytokine signaling (SOCS) protein.

In some embodiments, an immune cell activating agent comprises or is a costimulatory antibody or antigen binding fragment thereof, small molecule, polypeptide, glycoprotein, or exogenous cell. In some embodiments, a costimulatory antibody or antigen binding fragment thereof binds to 4-1BB, CD3, CD40, CD28, OX40, GITR, CTLA-4, PD-1, PD-L1, PD-L2, TIM-3, TGF-β, LAG-3, CD39, or CD73. In some embodiments, a costimulatory antibody or antigen binding fragment thereof comprises or is OKT3. In some embodiments, a costimulatory small molecule binds to 4-1BB, CD3, CD40, CD28, OX40, GITR, CTLA-4, PD-1, PD-L1, PD-L2, TIM-3, TGF-β, LAG-3, CD39, or CD73. In some embodiments, a costimulatory polypeptide comprises or is a soluble HHLA2 polypeptide (e.g., a HHLA2 fusion polypeptide, e.g., a HHLA2 Fc fusion polypeptide) or a fragment thereof. In some embodiments, a costimulatory glycoprotein comprises or is a fibronectin protein or fragment thereof. In some embodiments, a costimulatory exogenous cell comprises or is an artificial antigen presenting cell.

In some embodiments, immune effector cells are isolated from peripheral blood mononuclear cells (PBMCs) or tumor cells. In some embodiments, a modified immune effector cells comprise or are NK cells and/or T cells. In some embodiments, T cells comprise or are CD4+ T cells and/or CD8+ T cells. In some embodiments, modified immune effector cells comprise at least one CAR.

In some embodiments, modified immune effector cells are administered to a subject within less than about 3 hours of contacting with at least one KIR3DL3 inhibitor. In some embodiments, modified immune effector cells are administered to a subject within less than about 1 minute, about 2 minutes, about 3 minutes, about 4 minutes, about 5 minutes, about 10 minutes, about 30 minutes, or about 45 minutes, about 1 hour, about 1.5 hours, about 2 hours, about 2.5 hours, or about 3 hours of contacting with at least one KIR3DL3 inhibitor.

In some embodiments, a population of modified immune effector cells and/or at least one KIR3DL3 inhibitor is administered parenterally. In some embodiments, parenteral administration is or comprises subcutaneous, intravenous, intramuscular, or intrasternal injection or infusion.

In some embodiments, the methods comprise sequential administration of a population of modified immune effector cells and at least one KIR3DL3 inhibitor. In some embodiments, (i) a population of modified immune effector cells are administered prior to administration of at least one KIR3DL3 inhibitor; or (ii) a population of modified immune effector cells are administered after administration of at least one KIR3DL3 inhibitor.

In some embodiments, co-administration of a population of modified immune effector cells and at least one KIR3DL3 inhibitor. In some embodiments, the methods comprise co-administration by injection.

In some embodiments, a subject has a cancer. In some embodiments, a subject has a solid tumor. In some embodiments, a solid tumor is or comprises one or more of a renal cancer, a bone cancer, a skin cancer, a breast cancer, a cervical cancer, a colorectal cancer, an endometrial cancer, a lung cancer, an ovarian cancer, a liver cancer, cholangiocarcinoma, or a thyroid cancer. In some embodiments, a subject has a hematological cancer. In some embodiments, hematological cancer comprises or is a leukemia or lymphoma. In some embodiments, leukemia comprises or is acute lymphocytic leukemia, acute myeloid leukemia, chronic myeloid leukemia, acute lymphoblastic leukemia, chronic lymphocytic leukemia, chronic leukemia, or acute leukemia. In some embodiments, lymphoma comprises or is Hodgkin lymphoma (HL), non-Hodgkin's lymphoma, lymphocytic lymphoma, or diffuse large B cell lymphoma (DLBCL). In some embodiments, a subject has a cancer that is resistant to a treatment comprising a cytokine agent.

In some embodiments, methods described herein further comprise determining expression of TMIGD2 and/or KIR3DL3 by modified immune effector cells. In some embodiments, methods described herein further comprise determining activation of immune effector cells. In some embodiments, methods described herein further comprise determining expression of CD25, CD69, CD137, CD16, CD56, CD96, CD226, KIR2DL5, and/or NKG2D.

In some embodiments, methods described herein further comprise formulating a population of modified immune cells into a composition for administration to a subject.

In another aspect, the disclosure provides methods of making a population of modified immune effector cells comprising: (i) contacting a population of immune effector cells with at least one immune cell activating agent, and (ii) contacting a population of immune effector cells with at least one KIR3DL3 inhibitor, thereby creating a population of modified immune effector cells.

In another aspect, the disclosure provides compositions comprising a population of modified immune effector cells, at least one immune cell activating agent, and at least one KIR3DL3 inhibitor.

In another aspect, the disclosure provides compositions comprising a population of modified immune effector cells and at least one KIR3DL3 inhibitor, wherein the immune effector cells were contacted with at least one immune cell activating agent.

In another aspect, the disclosure provides kits comprising at least one immune cell activating agent, at least one KIR3DL3 inhibitor, and instructions for use and/or administration.

In another aspect, the disclosure provides kits comprising a population of modified immune effector cells and at least one KIR3DL3 inhibitor, and instructions for use and/or administration, wherein the immune effector cells were contacted with at least one immune cell activating agent.

In another aspect, the disclosure provides anti-KIR3DL3 antibodies or antigen-binding fragments thereof, which are or comprise: (a) a heavy chain variable region (VH) comprising one, two, or three VH CDR sequences each with at least about 90% identity to a VH CDR of Table 1; and/or (b) a light chain variable region (VL) comprising one, two, or three VL CDR sequences each with at least about 90% identity to a VL CDR of Table 1. Such anti-KIR3DL3 antibodies or antigen-binding fragments thereof can be used in any aspect or embodiment described herein.

In some embodiments, an anti-KIR3DL3 antibody or antigen-binding fragment thereof is or comprises: (a) a VH comprising one, two, or three VH CDR sequences each with at least about 95%, 96%, 97%, 98%, 99%, 99.5% or higher identity to a VH CDR of Table 1; and/or (b) a VL comprising one, two, or three VL CDR sequences each with at least about 95%, 96%, 97%, 98%, 99%, 99.5% or higher identity to a VL CDR of Table 1. In some embodiments, an anti-KIR3DL3 antibody or antigen-binding fragment thereof is or comprises: (a) a VH comprising one, two, or three VH CDR sequences each comprising or consisting of a VH CDR of Table 1; and/or (b) a VL comprising one, two, or three VL CDR sequences each comprising or consisting of a VL CDR of Table 1.

In some embodiments, an anti-KIR3DL3 antibody or antigen-binding fragment thereof is or comprises: (a) a VH with at least about 90% or more identity to a VH of Table 1; and/or (b) a VL with at least about 90% or more identity to a VL of Table 1. In some embodiments, an anti-KIR3DL3 antibody or antigen-binding fragment thereof is or comprises: (a) a VH with at least about 95%, 96%, 97%, 98%, 99%, 99.5% or higher identity to a VH of Table 1; and/or (b) a VL with at least about 95%, 96%, 97%, 98%, 99%, 99.5% or higher identity to a VL of Table 1. In some embodiments, an anti-KIR3DL3 antibody or antigen-binding fragment thereof is or comprises: (a) a VH comprising or consisting of a VH of Table 1; and/or (b) a VL comprising or consisting of a VL of Table 1.

In some embodiments, an anti-KIR3DL3 antibody or antigen-binding fragment thereof is or comprises: (a) a heavy chain with at least about 90% or more identity to a heavy chain of Table 1; and/or (b) a light chain with at least about 90% or more identity to a light chain of Table 1. In some embodiments, an anti-KIR3DL3 antibody or antigen-binding fragment thereof is or comprises: (a) a heavy chain with at least about 95%, 96%, 97%, 98%, 99%, 99.5% or higher identity to a heavy chain of Table 1; and/or (b) a light chain with at least about 95%, 96%, 97%, 98%, 99%, 99.5% or higher identity to a light chain of Table 1. In some embodiments, an anti-KIR3DL3 antibody or antigen-binding fragment thereof is or comprises: (a) a heavy chain comprising or consisting of a heavy chain of Table 1; and/or (b) a light chain comprising or consisting of a light chain of Table 1.

In another aspect, the disclosure provides nucleic acids encoding anti-KIR3DL3 antibodies or antigen-binding fragments thereof of any aspect or embodiment described herein.

In another aspect, the disclosure provides expression vectors comprising a nucleic acid of any aspect or embodiment described herein.

In another aspect, the disclosure provides host cells comprising or expressing an anti-KIR3DL3 antibody or antigen-binding fragment thereof of any aspect or embodiment described herein, comprising a nucleic acid of any aspect or embodiment described herein, or comprising an expression vector of any aspect or embodiment described herein.

In another aspect, the disclosure provides pharmaceutical compositions comprising at least one anti-KIR3DL3 antibody or antigen-binding fragment thereof of any aspect or embodiment described herein, and a pharmaceutically acceptable carrier, diluent, or excipient.

In another aspect, the disclosure provides methods of treating a subject having a disease, disorder, or condition comprising: administering a therapeutically effective amount of a pharmaceutical composition of any aspect or embodiment described herein.

In another aspect, the disclosure provides methods of modulating an immune response in a subject comprising: administering a therapeutically effective amount of a pharmaceutical composition of any aspect or embodiment described herein.

In some embodiments, a subject has or is at risk of developing a cancer. In some embodiments, a subject has a solid tumor or a hematological cancer. In some embodiments, a solid tumor is or comprises one or more of: a renal cancer, a bone cancer, a skin cancer, a breast cancer, a cervical cancer, a colorectal cancer, an endometrial cancer, a lung cancer, an ovarian cancer, a liver cancer, cholangiocarcinoma, or a thyroid cancer. In some embodiments, a hematological cancer comprises or is a leukemia or lymphoma. In some embodiments, a leukemia comprises or is acute lymphocytic leukemia, acute myeloid leukemia, chronic myeloid leukemia, acute lymphoblastic leukemia, chronic lymphocytic leukemia, chronic leukemia, or acute leukemia. In some embodiments, a lymphoma comprises or is Hodgkin lymphoma (HL), non-Hodgkin's lymphoma, lymphocytic lymphoma, or diffuse large B cell lymphoma (DLBCL).

### BRIEF DESCRIPTION OF THE DRAWING

The Figures described below, which together make up the Drawing, are for illustration purposes only, not for limitation.

FIGS. 1A-1B are schematics of the HHLA2 checkpoint axis showing that HHLA2 expressed on tumor cells modulates NK and T cell activity via interaction with KIR3DL3 or TMIGD2.

FIG. 2 is a graph showing monovalent binding affinity of an anti-KIR3DL3 antibody described herein (NPX267) to recombinant KIR3DL3 protein as determined by SPR using a Biacore instrument.

FIGS. 3A-3C are graphs showing binding of NPX267 to KIR3DL3 expressed on 300.19-KIR3DL3 cells (FIG. 3A), NK92MI cells (FIG. 3B), and primary human NK cells (FIG. 3C) by flow cytometry using an anti-human Phycoerythrin (PE) secondary antibody.

FIG. 4 is a series of graphs showing binding of NPX267 to KIR3DL3 expressed on tumor infiltrating CD56+ NK cells.

FIG. 5 is a graph showing the percentage (%) of KIR3DL3 binding to HHLA2 after 300.19-KIR3DL3 cells were treated with NPX267 or IgG4 isotype control antibody at concentrations ranging from 10 mg/mL to 0.0005 mg/mL.

FIG. 6 is a graph showing fold induction of luminescence in a T cell reporter assay of HHLA2/TCR/CHO cells and Jurkat/IL-2/KIR3DL3 cells pre-complexed with NPX267 and an anti-CD28 agonist antibody as determined using a luminometer (BioTek Synergy™ 2 microplate reader).

FIGS. 7A-7B are graphs showing NK92MI effector cell killing of K562 cells (FIG. 7A) or KIR3DL3+ human NK cell killing of HCC827 cells (FIG. 7B) following treatment with NPX267 or an IgG4 isotype control as assessed using flow cytometry.

FIG. 8 is a graph showing tumor growth, as assessed by imaging, in NSG mice injected intraperitoneally with luciferase tagged HCC827 cells and, after tumors were established, injected with KIR3DL3⁺ primary human NK cells, followed by either NPX267 parent Ab (26E10) or mIgG1 every other day for a total of 5 injections.

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Agent: As used herein, the term “agent” refers to a molecule that may be delivered to, or expressed, released, or secreted from, a target by an immune effector cell described herein. An agent includes, but is not limited to, a cytokine agent (e.g., a cytokine (IL-2, IL-15, IL-12, IL-12, IL-17, and/or IL-18)), a nucleic acid, an antibiotic, an anti-inflammatory agent, an antibody or fragments thereof, a chimeric antigen receptor, an antibody agent or fragments thereof, a glycoprotein, an artificial antigen presenting cell, a growth factor, an enzyme, a protein (e.g., an RNAse inhibitor), a peptide, a fusion protein, a synthetic molecule, an organic molecule (e.g., a small molecule), a carbohydrate, a lipid, a hormone, a microsome, a derivative or a variation thereof, and any combinations thereof. An agent may bind any cell moiety, such as a receptor, an antigenic determinant, or other binding site present on a target or target cell. An agent may diffuse or be transported into a cell, where it may act intracellularly.

Immunotherapeutic agent: The term “immunotherapeutic agent” can include any molecule, peptide, antibody, or other agent which can stimulate a host immune system to generate an immune response to a tumor or cancer in the subject. Various immunotherapeutic agents are useful in the compositions and methods described herein.

Affinity matured (or “affinity matured antibody”): as used herein, refers to an antibody with one or more alterations in one or more CDRs thereof which result an improvement in the affinity of the antibody for antigen, compared to a parent antibody which does not possess those alteration(s). In some embodiments, affinity matured antibodies will have nanomolar or even picomolar affinities for a target antigen. Affinity matured antibodies may be produced by any of a variety of procedures known in the art. Marks et al., BioTechnology 10:779-783 (1992) describes affinity maturation by VH and VL domain shuffling. Random mutagenesis of CDR and/or framework residues is described by: Barbas et al. Proc. Nat. Acad. Sci. U.S.A 91:3809-3813 (1994): Schier et al., Gene 169:147-155 (1995): Yelton et al., J. Immunol. 155:1994-2004 (1995): Jackson et al., J. Immunol. 154 (7): 3310-9 (1995); and Hawkins et al., J. Mol. Biol. 226:889-896 (1992).

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains—an amino-terminal variable (VL) domain, followed by a carboxy-terminal constant (CL) domain, separated from one another by another “switch”. Intact antibody tetramers comprise two heavy chain-light chain dimers in which the heavy and light chains are linked to one another by a single disulfide bond; two other disulfide bonds connect the heavy chain hinge regions to one another, so that the dimers are connected to one another and a tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including, for example, effector cells that mediate cytotoxicity. Affinity and/or other binding attributes of Fc regions for Fc receptors can be modulated through glycosylation or other modification. In some embodiments, antibodies produced and/or utilized in accordance with the present disclosure include glycosylated Fc domains, including Fc domains with modified or engineered glycosylation. In some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal. In some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody”, as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies: bi- or multi-specific antibodies (e.g., Zybodies®, etc): antibody fragments such as is used herein in the broadest sense and encompasses various antibody structures, including but not limited to monoclonal antibodies, polyclonal antibodies, multispecific antibodies (e.g., bispecific antibodies), and/or antibody fragments (preferably those fragments that exhibit the desired antigen-binding activity). An antibody described herein can be an immunoglobulin, heavy chain antibody, light chain antibody, LRR-based antibody, or other protein scaffold with antibody-like properties, as well as other immunological binding moiety known in the art, including, e.g., a Fab, Fab′, Fab′2, Fab2, Fab3, F(ab)2, Fd, Fv, Feb, scFv, SMIP, antibody, diabody, triabody, tetrabody, minibody, maxibody, tandab, DVD, BiTe, TandAb, or the like, or any combination thereof. The subunit structures and three-dimensional configurations of different classes of antibodies are known in the art. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.].

Antigen-binding fragment: An “antigen-binding fragment” refers to a portion of an intact antibody that binds the antigen to which the intact antibody binds. An antigen-binding fragment of an antibody includes any naturally occurring, enzymatically obtainable, synthetic, or genetically engineered polypeptide or glycoprotein that specifically binds an antigen to form a complex. Exemplary antibody fragments include, but are not limited to, Fv, Fab, Fab′, Fab′-SH, F(ab′)2; diabodies; linear antibodies; single-chain antibody molecules (e.g. scFv or VHH or VH or VL domains only); and multispecific antibodies formed from antibody fragments. In some embodiments, the antigen-binding fragments of the antibodies described herein are scFvs. In some embodiments, the antigen-binding fragments of the antibodies described herein are VHH domains only. As with full antibody molecules, antigen-binding fragments may be mono-specific or multispecific (e.g., bispecific). A multispecific antigen-binding fragment of an antibody may comprise at least two different variable domains, wherein each variable domain is capable of specifically binding to a separate antigen or to a different epitope of the same antigen.

Antigen presenting cell: As used herein, the terms “antigen presenting cell” or APC include professional antigen presenting cells (e.g., B lymphocytes, monocytes, dendritic cells, Langerhans cells) in addition to other antigen presenting cells (e.g., keratinocytes, endothelial cells, astrocytes, fibroblasts, and oligodendrocytes).

Antibody heavy chain: As used herein, the term “antibody heavy chain” refers to the larger of the two types of polypeptide chains present in all antibody molecules in their naturally occurring conformations.

Antibody light chain: As used herein, the term “antibody light chain” refers to the smaller of the two types of polypeptide chains present in all antibody molecules in their naturally occurring conformations.

Activation: As used herein, the term “activation” refers to the state of a cell, for example an immune effector cell described herein, that has been sufficiently stimulated to induce detectable cellular proliferation or has been stimulated to exert its effector function. Activation can also be associated with induced proliferation, cytokine production, cytokine secretion, cell signaling (e.g., gene expression changes), target cell killing, metabolic changes, production of inflammatory mediators, and/or antigen processing and presentation.

Synthetic antibody: As used herein, the term “synthetic antibody” refers to an antibody that is generated using recombinant DNA technology, such as, for example, an antibody expressed by a bacteriophage described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Antigen: As used herein, the term “antigen” or “Ag” refers to a molecule that is capable of provoking an immune response. This immune response may involve either antibody production, the activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequence or a partial nucleotide sequence encoding a protein that elicits an immune response encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

Autologous: As used herein, the term “autologous” refers to any material derived from an individual to which it is later to be re-introduced into the same individual.

Allogeneic: As used herein, the term “allogeneic” refers to any material (e.g., a population of cells) derived from a different animal of the same species.

Xenogeneic: As used herein, the term “xenogeneic” refers to any material (e.g., a population of cells) derived from an animal of a different species.

Chimeric Antigen Receptor: As used herein, the terms “Chimeric Antigen Receptor” or “CAR” refers to an artificial cell surface receptor that is engineered to be expressed on an immune effector cell described herein and specifically targets a cell and/or binds an antigen. CARs may be used, for example, as a therapy with adoptive cell transfer. A CAR can comprise at least one extracellular domain, at least one transmembrane domain, and at least one intracellular domain.

Cancer: As used herein, the term “cancer” refers to a disease characterized by the rapid and uncontrolled growth of aberrant cells. Cancer cells can spread locally or through the bloodstream and lymphatic system to other parts of the body. Cancers can include both solid tumors and hematological cancers. Examples of various cancers are described herein and include, but are not limited to, a renal cancer, a bone cancer, a skin cancer, a breast cancer, a cervical cancer, a colorectal cancer, an endometrial cancer, a lung cancer, an ovarian cancer, a liver cancer, cholangiocarcinoma, a thyroid cancer, leukemia, or lymphoma as well as several other types including those as described elsewhere herein. In certain embodiments, the cancer is renal cell carcinoma (RCC).

CDR: as used herein, refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Certain systems have been established in the art for defining CDR boundaries (e.g., Kabat, Chothia, etc.); those skilled in the art appreciate the differences between and among these systems and are capable of understanding CDR boundaries to the extent required to understand and to practice the claimed invention.

Chemotherapeutic Agent: The term “chemotherapeutic agent,” as used herein has its art-understood meaning referring to one or more pro-apoptotic, cytostatic and/or cytotoxic agents, for example specifically including agents utilized and/or recommended for use in treating one or more diseases, disorders or conditions associated with undesirable cell proliferation. In many embodiments, chemotherapeutic agents are useful in the treatment of cancer. In some embodiments, a chemotherapeutic agent may be or comprise one or more alkylating agents, one or more anthracyclines, one or more cytoskeletal disruptors (e.g. microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinum-based agents, one or more retinoids, one or more vinca alkaloids, and/or one or more analogs of one or more of the following (i.e., that share a relevant anti-proliferative activity). In some embodiments, a chemotherapeutic agent may be or comprise one or more of Actinomycin. All-trans retinoic acid, an Auiristatin, Azacitidine, Azathioprine, Bleomycin. Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fluorouracil, Gemcitabine. Hydroxyurea, Idarubicin, Imatinib, Irinotecan. Maytansine and/or analogs thereof (e.g. DM1) Mechlorethamine, Mercaptopurine, Methotrexate, Mitoxantrone, a Maytansinoid, Oxaliplatin, Paclitaxel, Pemetrexed, Teniposide. Tioguanine, Topotecan, Valrubicin, Vinblastine. Vincristine. Vindesine. Vinorelbine, or combinations thereof. In some embodiments, a chemotherapeutic agent may be utilized in the context of an antibody-drug conjugate. In some embodiments, a chemotherapeutic agent is an antibody-drug conjugate comprising: hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, and/or lorvotuzumab mertansine.

Conservative sequence modifications: As used herein, the term “conservative sequence modifications” refers to amino acid modifications that do not significantly affect or alter the binding characteristics of an antibody containing the amino acid sequence. Such conservative modifications include amino acid substitutions, additions and deletions. Modifications can be introduced into an antibody compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

Combination therapy: The term “combination therapy”, as used herein, refers to those situations in which two or more different therapeutic agents (e.g., a population of modified immune effector cells described herein and at least one KIR3DL3 inhibitor described herein) are administered in overlapping regimens so that the subject is simultaneously exposed to both agents. When used in combination therapy, two or more different therapeutic agents may be administered simultaneously or separately. This administration in combination can include simultaneous administration of the two or more therapeutic agents in the same dosage form, simultaneous administration in separate dosage forms, and separate administration. That is, two or more therapeutic agents can be formulated together in the same dosage form and administered simultaneously. Alternatively, two or more therapeutic agents can be simultaneously administered, wherein the agents are present in separate formulations. In another alternative, a first therapeutic agent can be administered followed by one or more additional therapeutic agents. In the separate administration protocol, two or more therapeutic agents may be administered a few minutes apart, or a few hours apart, a few days apart, or a few weeks apart. In some embodiments, two or more therapeutic agents may be administered within hours (e.g., less than about 3 hours) apart.

Composition: Those skilled in the art will appreciate that the term “composition” may be used to refer to a discrete physical entity that comprises one or more specified components. In general, unless otherwise specified, a composition may be of any form—e.g., gas, gel, liquid, or solid.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step

Concurrent administration: As used herein, the term “concurrent administration” with respect to two or more therapeutic agents described herein (e.g., a population of immune effector cells described herein and at least one KIR3DL3 inhibitor described herein), is administration performed using doses and time intervals such that the administered therapeutic agents are present together within the body, e.g., at one or more sites of action in the body, over a time interval in non-negligible quantities. The time interval can be minutes (e.g., at least 1 minute, 1-30 minutes, 30-60 minutes), hours (e.g., at least 1 hour, 1-2 hours, 2-6 hours, 6-12 hours, 12-24 hours), days (e.g., at least 1 day, 1-2 days, 2-4 days, 4-7 days, etc.), or weeks (e.g., at least 1, 2, or 3 weeks, etc.). Accordingly, the therapeutic agents may, but need not be, administered together, e.g., as part of a single composition. In addition, the therapeutic agents may, but need not be, administered essentially simultaneously (e.g., within less than 5 minutes, or within less than 1 minute apart) or within a short time of one another (e.g., less than 1 hour, less than 30 minutes, less than 10 minutes, approximately 5 minutes apart). According to various embodiments of the disclosure, therapeutic agents administered within such time intervals may be considered to be administered at substantially the same time. In certain embodiments of the disclosure, concurrently administered therapeutic agents are present at effective concentrations within the body over the time interval. When administered concurrently, the effective concentration of each of the therapeutic agents needed to elicit a particular biological response may be less than the effective concentration of each therapeutic agent when administered alone, thereby allowing a reduction in the dose of one or more of the therapeutic agents relative to the dose that would be needed if the agent was administered as a single agent. The effects of multiple therapeutic agents may, but need not be, additive or synergistic. The therapeutic agents may be administered multiple times.

Conservative sequence modifications: As used herein, the term “conservative sequence modifications” refers to amino acid modifications that do not significantly affect or alter the binding characteristics of an antibody or antigen-binding fragment thereof containing the amino acid sequence. Such conservative modifications include amino acid substitutions, additions, and deletions. Modifications can be introduced into an antibody compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

Cytotoxic: As used herein, the term “cytotoxic” or “cytotoxicity” refers to killing or damaging cells. In one embodiment, cytotoxicity of the metabolically enhanced cells is improved, e.g. increased cytolytic activity of immune effector cells described herein (e.g., T cells or NK cells).

Effective amount: As used herein, an “effective amount” described herein refers to a dose that is adequate to prevent or treat at least one sign and/or symptom of cancer in an individual. Amounts effective for a therapeutic or prophylactic use will depend on, for example, the stage and severity of the disease, disorder or condition being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations. For purposes of the disclosure, the amount or dose of a therapeutic agent (e.g., a modified immune effector cell described herein and/or at least one KIR3DL3 inhibitor described herein) administered should be sufficient to effect a therapeutic or prophylactic response in a subject over a reasonable time frame (e.g., reduction or other lessening of severity or duration of at least one sign or symptom). For example, the dose should be sufficient to detect, treat or prevent cancer in a period of from about 2 hours or longer, e.g., about 12 to about 24 or more hours, from the time of administration. In certain embodiments, the time period could be even longer. The dose will be determined by the efficacy of the particular one or more therapeutic agents and the condition of the subject (e.g., human), as well as the body weight of the subject (e.g., human) to be treated.

Effector function: As used herein, “effector function” or “effector activity” refers to a specific activity carried out by an immune cell in response to stimulation of the immune cell. For example, an effector function of a T lymphocyte includes, recognizing an antigen and killing a cell that expresses the antigen.

Epitope: As used herein, the term “epitope” refers to any moiety that is specifically recognized by an immunoglobulin (e.g., antibody or receptor) binding component. In some embodiments, an epitope is comprised of a plurality of chemical atoms or groups on an antigen. In some embodiments, such chemical atoms or groups are surface-exposed when the antigen adopts a relevant three-dimensional conformation. In some embodiments, such chemical atoms or groups are physically near to each other in space when the antigen adopts such a conformation. In some embodiments, at least some such chemical atoms are groups are physically separated from one another when the antigen adopts an alternative conformation (e.g., is linearized).

“Framework” or “framework region”: as used herein, refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FR1, for example, represents the first framework region closest to the amino terminal end of the variable region and 5′ with respect to CDR1, and FRs represents two or more of the sub-regions constituting a framework region.

Immune effector function: As used herein, “immune effector function” or “immune effector response,” as that term is used herein, refers to a function or response, e.g., of an immune effector cell described herein (e.g., a T cell or an NK cell), that enhances or promotes an immune attack of a target cell. For example, an immune effector function or response refers to a property of a T cell and/or NK cell that promotes the inhibition of growth or proliferation of a target cell. For example, in the case of a T cell, primary stimulation and co-stimulation are examples of immune effector function or response.

Encoding: As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Endogenous: As used herein “endogenous” refers to any material from or produced inside a particular organism, cell, tissue or system.

Exogenous: As used herein, the term “exogenous” refers to any material introduced from or produced outside a particular organism, cell, tissue or system.

Expand: As used herein, the term “expand” refers to increasing in number, as in an increase in the number of cells, for example, immune effector cells described herein, e.g., T cells or NK cells. In one embodiment, immune cells that are expanded ex vivo increase in number relative to the number originally present in a culture. In another embodiment, immune cells, that are expanded ex vivo increase in number relative to other cell types in a culture. In some embodiments, expansion may occur in vivo. The term “ex vivo,” as used herein, refers to cells that have been removed from a living organism. (e.g., a human) and propagated outside the organism (e.g., in a culture dish, test tube, or bioreactor).

Expression: As used herein, the term “expression” of a nucleic acid sequence refers to generation of any gene product from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription): (2) processing of an RNA transcript (e.g., by splicing, editing, 5′ cap formation, and/or 3′ end formation): (3) translation of an RNA into a polypeptide or protein; and/or (4) post-translational modification of a polypeptide or protein.

Fragment: As used herein, the terms “fragment” or “portion” refers to a structure that includes a discrete portion of the whole, but lacks one or more moieties found in the whole structure. In some embodiments, a fragment consists of such a discrete portion. In some embodiments, a fragment consists of or comprises a characteristic structural element or moiety found in the whole. In some embodiments, a nucleotide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., nucleic acids) as found in the whole nucleotide. In some embodiments, a nucleotide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in the whole nucleotide.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position: when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences.

Host cell: as used herein, the term “host cell” refers to a cell into which exogenous DNA (recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term host cell as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., or Streptomyces spp.), mycobacteria cells, fungal cells, yeast cells (e.g., S. cerevisiae, S. pombe, P. pastoris, or P. methanolica), plant cells, insect cells (e.g., SF-9, SF-21, baculovirus-infected insect cells, or Trichoplusia ni.), non-human animal cells, human cells, or cell fusions (e.g., hybridomas or quadromas). In some embodiments, the cell comprises or is a human, monkey, ape, hamster, rat, or mouse cell. In some embodiments, the cell is a eukaryotic cell chosen from: CHO (e.g., CHO K1, DXB-1 1 CHO, Veggie-CHO), COS (e.g., COS-7), retinal cell, Vero, CVI, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, or a cell line derived from an aforementioned cell. In some embodiments, a cell comprises one or more viral genes.

Human antibody: as used herein, the term “human antibody” is intended to include antibodies having variable and constant regions generated (or assembled) from human immunoglobulin sequences. In some embodiments, antibodies (or antibody components) may be considered to be “human” even though their amino acid sequences include residues or elements not encoded by human germline immunoglobulin sequences (e.g., include sequence variations, for example that may (originally) have been introduced by random or site-specific mutagenesis in vitro or by somatic mutation in vivo), for example in one or more CDRs and in particular CDR3.

Humanized: as is known in the art, the term “humanized” is commonly used to refer to antibodies (or antibody components) whose amino acid sequence includes VH and V_{L} region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody component) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab. Fab′, F(ab′)₂, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a C_{H}1, hinge, C_{H}2, C_{H}3, and, optionally, a C_{H}4 region of a heavy chain constant region. In some embodiments, a humanized antibody only contains a humanized VL region. In some embodiments, a humanized antibody only contains a humanized VH region. In some certain embodiments, a humanized antibody contains humanized VH and VL regions.

Identity: As used herein, the term “identity” refers to the subunit sequence identity between two polymeric molecules particularly between two amino acid molecules, such as, between two polypeptide molecules. When two amino acid sequences have the same residues at the same positions: e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions: e.g., if half (e.g., five positions in a polymer ten amino acids in length) of the positions in two sequences are identical, the two sequences are 50% identical: if 90% of the positions (e.g., 9 of 10), are matched or identical, the two amino acids sequences are 90% identical.

Substantial identity: As used herein, the term “substantial identity” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having an amino acid sequence at least 80%, preferably at least 85%, at least 90%, at least 95%, at least 98% or at least 99% identical to that of a reference CDR.

Immune cell: As used herein, the term “immune cell.” refers to a cell that is involved in an immune response, e.g., promotion of an immune response. Examples of immune cells include, but are not limited to, T cells, natural killer (NK) cells, macrophages, monocytes, dendritic cells, neutrophils, eosinophils, mast cells, platelets, large granular lymphocytes, Langerhans' cells, or B-lymphocytes. A source of immune cells (e.g., T cells or NK cells) can be obtained from a subject.

Immune checkpoint: As used herein, the term “immune checkpoint” refers to a group of molecules on the cell surface of CD4+ and/or CD8+ T cells that fine-tune immune responses by down-modulating or inhibiting an anti-tumor immune response. Immune checkpoint proteins are well-known in the art and include, without limitation. KIR family receptors, HHLA2, CTLA-4, PD-1, VISTA, B7-H2, B7-H3, PD-L1, B7-H4, B7-H6, ICOS, HVEM, PD-L2, CD160, gp49B, PIR-B, TIM-1, TIM-3, TIM-4, LAG-3, GITR, 4-IBB, OX-40, BTLA, SIRPα (CD47), CD48, 2B4 (CD244), B7.1, B7.2, ILT-2, ILT-4, TIGIT, butyrophilins, and A2aR. The term further encompasses biologically active protein fragment, as well as nucleic acids encoding full-length immune checkpoint proteins and biologically active protein fragments thereof. In some embodiment, the term further encompasses any fragment according to homology descriptions provided herein.

Immune response: As used herein the term “immune response” refers to a cellular and/or systemic response to an antigen that occurs when lymphocytes identify antigenic molecules as foreign and induce the formation of antibodies and/or activate lymphocytes to remove the antigen.

Immunoglobulin: As used herein, the term “immunoglobulin” or “Ig,” refers to a class of proteins that function as antibodies. Antibodies expressed by B cells are sometimes referred to as a BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is an immunoglobulin that has no known antibody function, but may serve as an antigen receptor. IgE is an immunoglobulin that mediates immediate hypersensitivity by causing release of mediators from mast cells and basophils upon exposure to allergen.

Isolated: As used herein, the term “isolated” refers to something altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

“Improve,” “increase”, “inhibit” or “reduce”: As used herein, the terms “improve,” “increase,” “inhibit,” “reduce,” or grammatical equivalents thereof, indicate values that are relative to a baseline or other reference measurement. In some embodiments, an appropriate reference measurement is or comprises a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement is or comprises a measurement in comparable system known or expected to respond in a particular way, in presence of the relevant agent or treatment.

K_{D}: as used herein, the term “K_{D}” refers to the dissociation constant of a binding agent (e.g., an antibody or antigen-binding fragment thereof) from a complex with its partner (e.g., the epitope to which the antibody or antigen-binding fragment thereof binds). The term “K_{D},” as used herein, equals K_{off} divided by Kₒₙ.

K_{off}: as used herein, the term “K_{off}” refers to the off rate constant for dissociation of a binding agent (e.g., an antibody or antigen-binding fragment thereof) from a complex with its partner (e.g., the epitope to which the antibody or antigen-binding fragment thereof binds).

Kₒₙ: as used herein, the term “Kₒₙ” refers to the on rate constant for association of a binding agent (e.g., an antibody or antigen-binding fragment thereof) with its partner (e.g., the epitope to which the antibody or antigen-binding fragment thereof binds).

Modulating: As used herein the term “modulating,” refers to mediating a detectable increase or decrease in the level of a response and/or a change in the nature of a response in a subject compared with the level and/or nature of a response in the subject in the absence of a treatment or compound, and/or compared with the level and/or nature of a response in an otherwise identical but untreated subject. The term encompasses perturbing and/or affecting a native signal or response thereby mediating a beneficial therapeutic response in a subject, preferably, a human. Monoclonal Antibody: A “monoclonal antibody” or “mAb” refers to an antibody obtained from a population of substantially homogeneous antibodies, i.e., the individual antibodies comprising the population are identical and/or bind the same epitope, except for possible variant antibodies (e.g., containing naturally occurring mutations or arising during production of a monoclonal antibody preparation), such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

Nucleic acid: As used herein, the term “nucleic acid” refers to a polymer of at least three nucleotides. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a non-natural residue comprises one or more modified sugars (e.g., 2′-fluororibose, ribose. 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Operably linked: As used herein, the term “operably linked” refers to functional linkage between, for example, a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Pharmaceutically acceptable: As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition, or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject. Some examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soy bean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Polynucleotide: As used herein, the term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure: in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known: in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class: in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids: in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Recombinant: as used herein, is intended to refer to polypeptides that are designed, engineered, prepared, expressed, created, manufactured, and/or or isolated by recombinant means, such as polypeptides expressed using a recombinant expression vector transfected into a host cell, polypeptides isolated from a recombinant, combinatorial human polypeptide library (see, e.g., Hoogenboom, TIB Tech 15:62, 1997; Azzazy Clin. Biochem. 35:425, 2002: Gavilondo BioTechniques 29:128, 2002: Hoogenboom Immunology Today 21:371, 2000), antibodies isolated from an animal (e.g., a mouse) that is transgenic for human immunoglobulin genes (see, e.g., Taylor Nuc. Acids Res. 20:6287, 1992: Little Immunology Today 12:364, 2000; Kellermann Curr. Opin. Biotechnol 13:593, 2002; Murphy Proc. Natl Acad Sci USA 111:5153, 2104) or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source. For example, in some embodiments, a recombinant antibody polypeptide is comprised of sequences found in the germline of a source organism of interest (e.g., human, mouse, etc.). In some embodiments, a recombinant antibody has an amino acid sequence that resulted from mutagenesis (e.g., in vitro or in vivo, for example in a transgenic animal), so that the amino acid sequences of the VH and VL regions of the recombinant antibodies are sequences that, while originating from and related to germline VH and VL sequences, do not naturally exist within the germline antibody repertoire in vivo.

Signal transduction pathway: As used herein, the term “signal transduction pathway” refers to the biochemical relationship between a plurality of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

Single chain antibodies: As used herein, the term “single chain antibodies” refers to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442: Huston et al. (1988) Proc. Natl. Acad. Sci. USA 85:5879-5883: Ward et al. (1989) Nature 334:54454: Skerra et al. (1988) Science 242:1038-1041.

Small molecule: As used herein, the term “small molecule” refers to a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide: for example, in some embodiments, a small molecule is not a glycoprotein, proteoglycan, or glycolipid. In some embodiments, a small molecule is not a lipid. In some embodiments, a small molecule is a modulating agent (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent. Those of ordinary skill in the art, reading the present disclosure, will appreciate that certain small molecule compounds may be provided and/or utilized in any of a variety of forms such as, for example, crystal forms, salt forms, protected forms, pro-drug forms, ester forms, isomeric forms (e.g., optical and/or structural isomers), or isotopic forms. Those of skill in the art will appreciate that certain small molecule compounds have structures that can exist in one or more stereoisomeric forms. In some embodiments, such a small molecule may be utilized in accordance with the present disclosure in the form of an individual enantiomer, diastereomer or geometric isomer, or may be in the form of a mixture of stereoisomers: in some embodiments, such a small molecule may be utilized in accordance with the present disclosure in a racemic mixture form. Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms. Those of skill in the art will appreciate that certain small molecule compounds have structures that permit isotopic substitution (e.g., ²H or ³H for H; ¹¹C, ¹³C or ¹⁴C for ¹²C; ¹³N or ¹⁵N for ¹⁴N; ¹⁷O or ¹⁸O for 16O; ³⁶Cl for XXC; ¹⁸F for XXF; 131I for XXXI; etc). In some embodiments, such a small molecule may be utilized in accordance with the present disclosure in one or more isotopically modified forms, or mixtures thereof. In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form. In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound is considered a different form of the compound than a racemic mixture of the compound; a particular salt of a small molecule compound is considered a different form from another salt form of the compound: a preparation that contains only a form of the compound that contains one conformational isomer ((Z) or (E)) of a double bond is considered to a different form of the compound from one that contains the other conformational isomer ((E) or (Z)) of the double bond: or a preparation in which one or more atoms is a different isotope than is present in a reference preparation is considered to be a different form.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments a human subject is an adult, adolescent, or pediatric subject. In some embodiments, a subject is suffering from a disease, disorder or condition, e.g., a disease, disorder, or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with and/or displays one or more symptoms of a disease, disorder, and/or condition.

Target: As used herein, the term “target” refers to a cell, tissue, organ, or site within the body that is the subject of provided methods, systems, and/or compositions, for example, a cell, tissue, organ or site within a body that is in need of treatment or is preferentially bound by, for example, a population of modified immune effector cells described herein or a KIR3DL3 inhibitor described herein.

Therapeutic: As used herein, the term “therapeutic” refers to a treatment and/or prophylaxis. A therapeutic effect is obtained, for example, by suppression, remission, or eradication of a disease state.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent can be an agent that, when administered to a subject, can prevent an undesired side effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition. A therapeutic agent includes, but is not limited to, a population of modified immune effector cells described herein and/or at least one KIR3DL3 inhibitor described herein.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, and/or the target cell or tissue. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose. In some embodiments, multiple unit doses are required to deliver a therapeutically effective amount.

Treat: As used herein, the term “treat,” “treatment,” or “treating” refers to partial or complete alleviation, amelioration, delay of onset of, inhibition, prevention, relief, and/or reduction in incidence and/or severity of one or more symptoms or features of a disease, disorder, and/or condition. In some embodiments, treatment may be administered to a subject who does not exhibit signs or features of a disease, disorder, and/or condition (e.g., may be prophylactic). In some embodiments, treatment may be administered to a subject who exhibits only early or mild signs or features of the disease, disorder, and/or condition, for example for the purpose of decreasing the risk of developing pathology associated with the disease, disorder, and/or condition. In some embodiments, treatment may be administered to a subject who exhibits established, severe, and/or late-stage signs of the disease, disorder, or condition. In some embodiments, treating may comprise administering a population of modified immune effector cell described herein (e.g., a T cell or an NK cell) and/or at least one KIR3DL3 inhibitor described herein to a subject.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DETAILED DESCRIPTION

Provided herein, amongst other things, are anti-KIR3DL3 antibodies and antigen-binding fragments thereof. In some embodiments, anti-KIR3DL3 antibodies and antigen-binding fragments thereof described herein specifically bind KIR3DL3 (e.g., human KIR3DL3) with high affinity. In some embodiments, anti-KIR3DL3 antibodies and antigen-binding fragments thereof described herein block binding of KIR3DL3 to HHLA2. In some embodiments, anti-KIR3DL3 antibodies and antigen-binding fragments thereof described herein specifically bind to KIR3DL3 expressed on NK cells. In some embodiments, anti-KIR3DL3 antibodies and antigen-binding fragments thereof described herein block HHLA2-mediated suppressive activity in T cells. In some embodiments, anti-KIR3DL3 antibodies and antigen-binding fragments thereof described herein enhance NK cell killing of HHLA2-expressing tumor cells. In some embodiments, anti-KIR3DL3 antibodies and antigen-binding fragments thereof described herein enhance anti-tumor activity. Accordingly, the present disclosure provides anti-KIR3DL3 antibodies and antigen-binding fragments thereof described herein that can be useful for methods of treating diseases, disorders, and conditions, such as various cancers described herein, and for methods of modulating an immune response.

### Immune Effector Cells

The present disclosure, among other things, provides immune effector cells described herein (e.g., NK cells or T cells) modified with at least one immune cell activating agent and/or at least one KIR3DL3 inhibitor to produce a population of modified immune effector cells. Accordingly, in some embodiments, a population of modified immune effector cells exhibits enhanced proliferation, e.g., relative to proliferation prior to modification. In some embodiments, a population of modified immune effector cells exhibits increased endogenous expression of at least one cytokine, e.g., relative to endogenous expression of at least one cytokine prior to modification. Methods of making a population of modified immune effector cells described herein can include: (i) contacting a population of immune effector cells with at least one immune cell activating agent described herein, and/or (ii) contacting the population of immune effector cells with at least one KIR3DL3 inhibitor described herein.

As used herein, the term “immune effector cell” refers to a cell that is involved in an immune response, e.g., promotion of an immune response. Examples of immune cell effector cells include, but are not limited to, natural killer (NK) cells, T cells (e.g., alpha/beta T cells or gamma/delta T cells), natural killer T (NKT) cells, B cells, mast cells, and myeloid-derived phagocytes.

As used herein, the terms “modified” or modification” refer to a changed state or structure of a cell (e.g., an immune effector cell described herein) or a molecule described herein. Cells may be modified through the introduction of one or more agents described herein (e.g., at least one immune cell activating agent described herein or at least one KIR3DL3 inhibitor described herein). Molecules may be modified in many ways, including chemically, structurally, and functionally. In some embodiments, a modified immune effector cell has improved effector function as a result of modification, e.g., an immune effector cell contacted with at least one immune cell activating agent described herein (e.g., a cytokine agent described herein) and/or at least one KIR3DL3 inhibitor described herein. In some embodiments, the at least one KIR3DL3 inhibitor is one or more anti-KIR3DL3 antibodies or antigen-binding fragments thereof described herein.

### T Cells

In some embodiments, immune effector cells comprise or are T cells. T cells can have effector functions (Teffs) to increase immune responses by expression of one or more T cell receptors (TCRs). In some embodiments, effector functions comprise or are one or more of cytokine secretion, cytotoxic activity, and/or anti-self-recognition. Conventional T cells (Tconv or Teffs) can be any T cell population that is not a T regulatory cell (Treg) and include, but are not limited to, naive T cells, activated T cells, memory T cells, resting Tcons, or Tcons that have differentiated, e.g., into Th1 or Th2 lineages. In some embodiments, Teffs comprise or are CD4+ Teffs, such as CD4+ helper T cells (e.g., ThO, Thl, Tfh, or Thl 7). In some embodiments, Teffs comprise or are CD8+ cytotoxic T cells. In some embodiments, Teffs comprise or are a subset of non-Treg T cells. In some embodiments, cytotoxic T cells are CD8+ T lymphocytes.

Prior to modification of a T cell described herein, a source of T cells can be obtained from a subject. T cells can be obtained from a number of sources, including peripheral blood mononuclear cells (PBMCs), bone marrow, lymph node tissue, cord blood, thymus tissue, tissue from a site of infection, ascites, pleural effusion, spleen tissue, and tumors. In some embodiments, T cells can be obtained from a unit of blood collected from a subject using any number of techniques known to the skilled artisan, such as Ficoll™ separation or magnetic bead separation. Any number of T cell lines available in the art, may also be used.

T cells may be expanded by any method known in the art, such as by contacting T cells with a surface having attached thereto an agent that stimulates a CD3/TCR complex associated signal and a ligand that stimulates a costimulatory molecule on the surface of the T cells. T cell populations may be stimulated using a variety of methods known in the art, such as by contact with an anti-CD3 antibody, or antigen-binding fragment thereof. For co-stimulation of an accessory molecule on the surface of the T cells, a ligand that binds the accessory molecule may be used. For example, an anti-CD28 antibody may mimic the activity of B7-1 or B7-2 by activating naïve T cells via CD28 prior to translocation of CTLA4 to the cell surface and subsequent T cell suppression. In some embodiments, a population of T cells (e.g., CD4+ T cells or CD8+ T cells) can be contacted with an anti-CD3 antibody and an anti-CD28 antibody, under conditions appropriate for stimulating proliferation of the T cells. In some embodiments, a population of T cells (e.g., CD4+ T cells or CD8+ T cells) can be contacted with an effective amount of a cytokine (e.g., IL-2 or IL-15), under conditions appropriate for stimulating proliferation of the T cells.

### NK Cells

In some embodiments, immune effector cells comprise or are natural killer (NK cells). NK cells can exhibit cytolytic activity against a variety of targets via exocytosis of cytoplasmic granules containing a variety of proteins, including perforin and granzyme proteases. Killing by NK cells can be triggered in a contact-dependent, non-phagocytotic process, which does not require prior sensitization to an antigen. Human NK cells can be characterized by the presence of the cell-surface markers CD16 and CD56, and the absence of the T cell receptor (CD3).

Mature NK cells, NK progenitor cells, or mixed populations of NK progenitor and mature NK cells can be used in the methods and compositions described herein. Mature NK cells comprise or are committed NK cells, having characteristic surface markers (e.g., CD16 and CD56) and NK cell function, and lacking the potential for further differentiation. NK progenitor cells may be derived from common lymphoid progenitors (CLPs).

Prior to modification of NK cells described herein, a source of NK cells can be obtained from a subject. NK cells can be obtained from a number of sources, including peripheral blood mononuclear cells (PBMCs), bone marrow, lymph node tissue, cord blood, thymus tissue, tissue from a site of infection, ascites, pleural effusion, spleen tissue, and tumors. In some embodiments, NK cells can be obtained from a unit of blood collected from a subject using any number of techniques known to the skilled artisan, such as Ficoll™ separation or magnetic bead isolation. Any number of NK cell lines (e.g., NK-92 cell line) available in the art, may also be used.

NK cells may be stimulated using a variety of methods known in the art, such as by incubating with an anti-CD20 antibody or co-culture with CD20-expressing cells. In some embodiments, activation statute of NK cell is determined by assessing expression of markers (e.g., CD25, CD69, CD137, CD16, CD56, CD96, CD226, TIGIT, KIR2DL5, and/or NKG2D). Cell surface expression of markers can be determined, for example, via FACS analysis or immunohistological staining techniques.

Antibody-dependent cell-mediated cytotoxicity (ADCC) may also be determined for NK cells described herein. ADCC refers to a form of cytotoxicity in which secreted antibodies bound onto Fc receptors (FcRs) present on cytotoxic cells (e.g. Natural Killer (NK) cells, neutrophils, and macrophages) enable these cytotoxic effector cells to bind specifically to an antigen-bearing target cell and subsequently kill the target cell. To assess ADCC activity of NK cells, an in vitro ADCC assay, such as that described in U.S. Pat. No. 5,500,362 or 5,821,337 may be performed.

Immune effector cells described herein may be activated and expanded generally using methods as described, for example, in U.S. Pat. No. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005, each of which is hereby incorporated by its reference in its entirety.

### Chimeric Antigen Receptors

In some embodiments, immune effector cells (e.g., T cells or NK cells) described herein may comprise at least one CAR. Accordingly, in some embodiments, an immune effector cell comprising at least one CAR comprises: (a) an extracellular domain (e.g., an extracellular domain described herein), (b) a transmembrane domain (e.g., a transmembrane domain described herein), and (c) an intracellular domain (e.g., an intracellular domain described herein).

In some embodiments, a CAR comprises an antigen binding domain that binds to an antigen, for example, on a target cell. In some embodiments, a tumor antigen comprises CD19; CD123: CD22: CD30; CD171; CS-1: C-type lectin-like molecule-1 (CLL-1 or CLECL1): CD33; epidermal growth factor receptor variant III (EGFRvIII): TNF receptor family member B cell maturation (BCMA): prostate-specific membrane antigen (PSMA); Receptor tyrosine kinase-like orphan receptor 1 (ROR1): Fms-Like Tyrosine Kinase 3 (FLT3): CD38: CD44v6; Carcinoembryonic antigen (CEA); Epithelial cell adhesion molecule (EPCAM): B7H3 (CD276); KIT (CD117); Interleukin-13 receptor subunit alpha-2 (IL-13Ra2 or CD213A2); Mesothelin; vascular endothelial growth factor receptor 2 (VEGFR2): Lewis (Y) antigen; or CD24.

In some embodiments, a CAR comprises one or more extracellular leader domains, one or more extracellular hinge domains and/or one or more intracellular co-stimulatory domains. In some embodiments, a CAR comprises a leader sequence at the N-terminus. In some embodiments, a CAR comprises a leader sequence at the N-terminus of the extracellular antigen recognition domain, wherein the leader sequence is optionally cleaved from the antigen recognition domain (e.g., a scFv) during cellular processing and localization of the CAR to the cellular membrane.

In some embodiments, a CAR comprises a transmembrane domain, for example, that connects an extracellular domain to an intracellular domain. In some embodiments, a transmembrane domain is naturally associated with one or more other domains of a CAR. In some embodiments, a transmembrane domain comprises one or more of the following: alpha, beta, or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, CD154. KIRDS2, OX40, ROR1, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, or NKG2C.

In some embodiments, a CAR comprises one or more intracellular domains. In some embodiments, an intracellular domain of a CAR comprises at least one domain responsible for signal activation and/or transduction. In some embodiments, an intracellular domain is or comprises at least one signaling domain. In some embodiments, an intracellular signaling domain comprises or is a functional signaling domain derived from one or more of TCR zeta, FcR gamma, FcR beta, CD3 zeta, CD3 gamma, CD3 delta, CD3 epsilon, CD5, CD22, CD79a, CD79b, and CD66d. In some embodiments, an intracellular signalling domain can further comprise a costimulatory signaling domain. A costimulatory signaling domain refers to a portion of a CAR comprising an intracellular domain of a costimulatory molecule. Examples of such costimulatory molecules include, but are not limited to, CD27, CD28, 4-1BB (CD137), OX40, CD30, CD40, PD-1 (also known as PD1), ICOS, LFA-1, CD2, CD7, LIGHT, NKG2C, and B7-H3.

### KIR3DL3 Inhibitors

The present disclosure, among other things, provides KIR3DL3 inhibitors. In some embodiments, a population of immune effector cells described herein is contacted with at least one KIR3DL3 inhibitor and at least one immune cell activating agent (e.g., prior to or substantially simultaneously with at least one KIR3DL3 inhibitor) to form a population of modified immune effector cells. In some embodiments, at least one KIR3DL3 inhibitor is administered to a subject in combination with a population of modified immune effector cells (e.g., immune effector cells contacted with at least one immune cell activating agent to form a population of modified immune effector cells prior to administering to a subject).

HHLA2 is a B7 family member that modulates NK cell and T cell functions. HHLA2 is broadly expressed in a variety of tumors and antigen presenting cells and has been implicated as both an activating and inhibitory ligand for NK cells and T cells. HHLA2 is a specific ligand for TMIGD2 and the interaction of HHLA2 and TMIGD2 selectively stimulates cell proliferation and cytokine production. HHLA2 also binds KIR3DL3, a receptor on T cells and NK cells, resulting in inhibition of T cell and NK cell activation. The present disclosure provides KIR3DL3 inhibitors for use in combination with immune cell activating agents for treating a variety of cancers, including solid tumors and hematological tumors.

The terms “KIR3DL3” or “Killer cell immunoglobulin-like receptor 3DL3,” as used herein, refer to a member of the Killer cell immunoglobulin-like receptor transmembrane glycoprotein family expressed by NK cells and T cells. KIR3DL3 is also known as KIRC1, CD158Z, KIR3DL7, and KIR44. The killer cell immunoglobulin-like receptor (KIR) genes are polymorphic and highly homologous genes found in a cluster on chromosome 19q13.4 within the 1 Mb leukocyte receptor complex (LRC). The gene content of the KIR gene cluster varies among haplotypes, although several “framework” genes are found in all haplotypes (KIR3DL3, KIR3DP1, KIR3DL4, and KIR3DL2). The KIR proteins are classified by the number of extracellular immunoglobulin domains (2D or 3D) and by whether they have a long (L) or short(S) cytoplasmic domain. KIR proteins with the long cytoplasmic domain transduce inhibitory signals upon ligand binding via an immune tyrosine-based inhibitory motif (ITIM), while KIR proteins with the short cytoplasmic domain lack the ITIM motif and instead associate with the TYRO protein tyrosine kinase binding protein to transduce activating signals. The ligands for several KIR proteins are subsets of HLA class I molecules; thus. KIR proteins are thought to play an important role in regulation of the immune response. The KIR3DL3 protein has an N-terminal signal sequence, 3 Ig domains, a transmembrane region lacking a positively charged residue, and a long cytoplasmic tail containing an ITIM. KIR3DL3 lacks the stalk region found in other KIRs.

The term “KIR3DL3” includes fragments, variants (e.g., allelic variants), and derivatives thereof. Representative human KIR3DL3 cDNA and human KIR3DL3 polypeptide sequences are publicly available from the National Center for Biotechnology Information (NCBI). For example, at least one human KIR3DL3 isoform is known: human KIR3DL3 (NM_153443.4) encoded by the transcript (NP_703144.3). Nucleic acid and polypeptide sequences of KIR3DL3 orthologs in organisms other than humans are also known including, but not limited to, chimpanzee KIR3DL3 (XM_003316679.3 and XP_003316727.3), Rhesus monkey KIR3DL3 (NM_001104552.2 and NP_001098022.1), mouse KIR3DL3 (NM_001310690.1 and NP_001297619.1, NM_177749.4 and NP_808417.2, NM_177748.2 and NP_808416.1), and rat KIR3DL3 (NM_181479.2 and NP_852144.1).

In some embodiments, a KIR3DL3 inhibitor exhibits binding affinity for KIR3DL3 or a fragment thereof (e.g., as assessed in a diagnostic assay, such as immunohistochemical (IHC), Western blot, intercellular flow, or ELISA). In some embodiments, a KIR3DL3 inhibitor exhibits the ability to inhibit KIR3DL3 binding to HHLA2.

### Anti-KIR3DL3 Antibodies and Antigen-Binding Fragments

Disclosed herein are methods, compositions, and formulations that include inhibitors of KIR3DL3, e.g., an anti-KIR3DL3 antibody or an antigen-binding fragment thereof. For example, an anti-KIR3DL3 antibody or fragment thereof may bind specifically to an epitope on KIR3DL3.

In some embodiments, an anti-KIR3DL3 antibody or antigen-binding fragment thereof comprises or is a monoclonal antibody. In some embodiments, an anti-KIR3DL3 antibody or antigen-binding fragment thereof comprises or is a full length antibody, e.g., comprising an immunoglobulin Fc region. In some embodiments, an anti-KIR3DL3 antibody or antigen-binding fragment thereof comprises or is a multispecific antibody, e.g., comprising a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, an anti-KIR3DL3 antibody or antigen-binding fragment thereof comprises or is a bispecific antibody molecule. In some embodiments, an antibody or antigen-binding fragment thereof is or has been affinity matured.

An anti-KIR3DL3 antibody or antigen-binding fragment thereof can include a heavy (H) chain variable domain sequence (VH), and a light (L) chain variable domain sequence (VL). In some embodiments, an anti-KIR3DL3 antibody or antigen-binding fragment thereof comprises or is a heavy chain and a light chain (a half antibody). In some embodiments, an anti-KIR3DL3 antibody or antigen-binding fragment thereof comprises or is two heavy (H) chain variable domain sequences and two light (L) chain variable domain sequence, thereby forming two antigen binding sites, such as Fab, Fab′, F(ab)2, Fc, Fd, Fd′, Fv, single chain antibodies (scFv), single variable domain antibodies, diabodies (Dab) (bivalent and bispecific), and chimeric (e.g., humanized) antibodies, which may be produced by the modification of whole antibodies or those synthesized de novo using recombinant DNA technologies. Such functional antibody fragments can retain the ability to selectively bind with KIR3DL3.

Examples of antigen-binding fragments of an anti-KIR3DL3 antibody can include: (i) a Fab fragment, a monovalent fragment consisting of the VL, VH, CL, and CH1 domains: (ii) a F(ab′)2 fragment, a bivalent fragment comprising two Fab fragments linked by a disulfide bridge at the hinge region: (iii) a Fd fragment consisting of the VH and CH1 domains: (iv) a Fv fragment consisting of the VL and VH domains of a single arm of an antibody, (v) a diabody (dAb) fragment, which consists of a VH domain: (vi) a camelid or camelized variable domain: (vii) a scFv; and (viii) a single domain antibody. Antibody fragments can be obtained using conventional techniques known to those of skill in the art, and the fragments are screened for utility in the same manner as intact antibodies. Anti-KIR3DL3 antibodies and antibody fragments can be from any class of antibodies including, but not limited to, IgG, IgA, IgM, IgD, and IgE, and from any subclass (e.g., IgG1, IgG2, IgG3, and IgG4) of antibodies. Preparation of an anti-KIR3DL3 antibody or antigen-binding fragment thereof can be monoclonal or polyclonal. An anti-KIR3DL3 antibody or antigen-binding fragment thereof can also be a human, humanized, CDR-grafted, or in vitro generated. An anti-KIR3DL3 antibody or fragment can have a heavy chain constant region chosen from, e.g., IgG1, IgG2, IgG3, or IgG4. An anti-KIR3DL3 antibody or antigen-binding fragment can also have a light chain chosen from, e.g., kappa or lambda. The term “immunoglobulin” (Ig) is used interchangeably with the term “antibody” herein.

VH and VL regions can be subdivided into regions of hypervariability, termed “complementarity determining regions” (CDRs), interspersed with regions that are more conserved, termed “framework regions” (FR or FW). The terms “complementarity determining region.” and “CDR.” as used herein, refer to the sequences of amino acids within antibody variable regions which confer antigen specificity and binding affinity. In general, there are three CDRs in each heavy chain variable region (HCDR1, HCDR2, and HCDR3) and three CDRs in each light chain variable region (LCDR1, LCDR2, and LCDR3). The extent of the framework region and CDRs can be precisely defined using a number of well-known schemes (see, e.g., Kabat, E. A., et al. (1991) Sequences of Proteins of Immunological Interest, Fifth Edition, U.S. Department of Health and Human Services, NIH Publication No. 91-3242: Chothia, C. et al. (1987) J. Mol. Biol. 196:901-917; and the AbM definition used by Oxford Molecular's AbM antibody modeling software, each of which is hereby incorporated by reference in its entirety).

In some embodiments, an anti-KIR3DL3 antibody or an antigen-binding fragment thereof comprises: (a) a heavy chain variable region (VH) comprising one, two, or three VH CDR sequences each with at least about 80%, 85%, 90%, 91%, 92%, 93%, 94%, 95%, 96%, 97%, 98%, 99%, 99.5% or more identity to a VH CDR in Table 1; and/or (b) a light chain variable region (VL) comprising one, two, or three VL CDR sequences each with at least about 80%, 85%, 90%, 91%, 92%, 93%, 94%, 95%, 96%, 97%, 98%, 99%, 99.5% or more identity to a VL CDR in Table 1. In some embodiments, an anti-KIR3DL3 antibody or an antigen-binding fragment thereof comprises: (a) a VH with at least about 80%, 85%, 90%, 91%, 92%, 93%, 94%, 95%, 96%, 97%, 98%, 99%, 99.5% or more identity to a VH in Table 1; and/or (a) a VL with at least about 80%, 85%, 90%, 91%, 92%, 93%, 94%, 95%, 96%, 97%, 98%, 99%, 99.5% or more identity to a VL in Table 1. In some embodiments, an anti-KIR3DL3 antibody or an antigen-binding fragment thereof comprises: (a) a heavy chain with at least about 80%, 85%, 90%, 91%, 92%, 93%, 94%, 95%, 96%, 97%, 98%, 99%, 99.5% or more identity to a heavy chain in Table 1; and/or (a) a light chain with at least about 80%, 85%, 90%, 91%, 92%, 93%, 94%, 95%, 96%, 97%, 98%, 99%, 99.5% or more identity to a light chain in Table 1.

In some embodiments, an anti-KIR3DL3 antibody or an antigen-binding fragment thereof described herein comprises: (a) a VH comprising a VH CDR1 amino acid sequence of SEQ ID NO: 1, a VH CDR2 amino acid sequence of SEQ ID NO: 2, and a VH CDR3 amino acid sequence of SEQ ID NO: 3; and (b) a VL comprising a VL CDR1 amino acid sequence of SEQ ID NO: 14, a VL CDR2 amino acid sequence of SEQ ID NO: 15, and a VL CDR3 amino acid sequence of SEQ ID NO: 16. In some embodiments, an anti-KIR3DL3 antibody or an antigen-binding fragment thereof described herein comprises: (a) a VH comprising a VH CDR1 amino acid sequence of SEQ ID NO: 4, a VH CDR2 amino acid sequence of SEQ ID NO: 5, and a VH CDR3 amino acid sequence of SEQ ID NO: 6; and (b) a VL comprising a VL CDR1 amino acid sequence of SEQ ID NO: 17, a VL CDR2 amino acid sequence of SEQ ID NO: 18, and a VL CDR3 amino acid sequence of SEQ ID NO: 19, each disclosed in Table 1. In some embodiments, an anti-KIR3DL3 antibody or an antigen-binding fragment thereof described herein comprises: (a) a VH comprising a VH CDR1 amino acid sequence of SEQ ID NO: 7, a VH CDR2 amino acid sequence of SEQ ID NO: 8, and a VH CDR3 amino acid sequence of SEQ ID NO: 9; and (b) a VL comprising a VL CDR1 amino acid sequence of SEQ ID NO: 20, a VL CDR2 amino acid sequence of SEQ ID NO: 21, and a VL CDR3 amino acid sequence of SEQ ID NO: 22, each disclosed in Table 1.

In some embodiments, an anti-KIR3DL3 antibody or an antigen-binding fragment thereof described herein comprises a VH comprising an amino acid sequence of SEQ ID NO: 10, or an amino acid sequence at least 85%, 90%, 95%, or 99% identical or higher to SEQ ID NO: 10. In some embodiments, an anti-KIR3DL3 antibody or an antigen-binding fragment thereof described herein comprises a VL comprising an amino acid sequence of SEQ ID NO: 23, or an amino acid sequence at least 85%, 90%, 95%, or 99% identical or higher to SEQ ID NO: 23. In some embodiments, an anti-KIR3DL3 antibody or an antigen-binding fragment thereof described herein comprises a VH comprising an amino acid sequence of SEQ ID NO: 10 and a VL comprising an amino acid sequence of SEQ ID NO: 23.

In some embodiments, an anti-KIR3DL3 antibody or an antigen-binding fragment thereof described herein comprises a heavy chain comprising an amino acid sequence of SEQ ID NO: 12, or an amino acid sequence at least 85%, 90%, 95%, or 99% identical or higher to SEQ ID NO: 12. In some embodiments, an anti-KIR3DL3 antibody or an antigen-binding fragment thereof described herein comprises a light chain comprising an amino acid sequence of SEQ ID NO: 25, or an amino acid sequence at least 85%, 90%, 95%, or 99% identical or higher to SEQ ID NO: 25. In some embodiments, an anti-KIR3DL3 antibody or an antigen-binding fragment thereof described herein comprises a heavy chain comprising an amino acid sequence of SEQ ID NO: 12 and a light chain comprising an amino acid sequence of SEQ ID NO: 25.

In some embodiments, an anti-KIR3DL3 antibody or an antigen-binding fragment thereof described herein comprises: (a) a VH comprising a VH CDR1 amino acid sequence of SEQ ID NO: 27, a VH CDR2 amino acid sequence of SEQ ID NO: 28, and a VH CDR3 amino acid sequence of SEQ ID NO: 29; and (b) a VL comprising a VL CDR1 amino acid sequence of SEQ ID NO: 40, a VL CDR2 amino acid sequence of SEQ ID NO: 41, and a VL CDR3 amino acid sequence of SEQ ID NO: 42. In some embodiments, an anti-KIR3DL3 antibody or an antigen-binding fragment thereof described herein comprises: (a) a VH comprising a VH CDR1 amino acid sequence of SEQ ID NO: 30, a VH CDR2 amino acid sequence of SEQ ID NO: 31, and a VH CDR3 amino acid sequence of SEQ ID NO: 32; and (b) a VL comprising a VL CDR1 amino acid sequence of SEQ ID NO: 43, a VL CDR2 amino acid sequence of SEQ ID NO: 44, and a VL CDR3 amino acid sequence of SEQ ID NO: 45. In some embodiments, an anti-KIR3DL3 antibody or an antigen-binding fragment thereof described herein comprises: (a) a VH comprising a VH CDR1 amino acid sequence of SEQ ID NO: 33, a VH CDR2 amino acid sequence of SEQ ID NO: 34, and a VH CDR3 amino acid sequence of SEQ ID NO: 35; and (b) a VL comprising a VL CDR1 amino acid sequence of SEQ ID NO: 46, a VL CDR2 amino acid sequence of SEQ ID NO: 47, and a VL CDR3 amino acid sequence of SEQ ID NO: 48.

In some embodiments, an anti-KIR3DL3 antibody or an antigen-binding fragment thereof described herein comprises a VH comprising an amino acid sequence of SEQ ID NO: 36, or an amino acid sequence at least 85%, 90%, 95%, or 99% identical or higher to SEQ ID NO: 36. In some embodiments, an anti-KIR3DL3 antibody or an antigen-binding fragment thereof described herein comprises a VL comprising an amino acid sequence of SEQ ID NO: 49, or an amino acid sequence at least 85%, 90%, 95%, or 99% identical or higher to SEQ ID NO: 49. In some embodiments, an anti-KIR3DL3 antibody or an antigen-binding fragment thereof described herein comprises a VH comprising an amino acid sequence of SEQ ID NO: 36 and a VL comprising an amino acid sequence of SEQ ID NO: 49.

In some embodiments, an anti-KIR3DL3 antibody or an antigen-binding fragment thereof described herein comprises a heavy chain comprising an amino acid sequence of SEQ ID NO: 38, or an amino acid sequence at least 85%, 90%, 95%, or 99% identical or higher to SEQ ID NO: 38. In some embodiments, an anti-KIR3DL3 antibody or an antigen-binding fragment thereof described herein comprises a light chain comprising an amino acid sequence of SEQ ID NO: 51, or an amino acid sequence at least 85%, 90%, 95%, or 99% identical or higher to SEQ ID NO: 51. In some embodiments, an anti-KIR3DL3 antibody or an antigen-binding fragment thereof described herein comprises a heavy chain comprising an amino acid sequence of SEQ ID NO: 38, and a light chain comprising an amino acid sequence of SEQ ID NO: 51.

In some embodiments, an anti-KIR3DL3 antibody or an antigen-binding fragment thereof described herein comprises: (a) a VH comprising a VH CDR1 amino acid sequence of SEQ ID NO: 53, a VH CDR2 amino acid sequence of SEQ ID NO: 54, and a VH CDR3 amino acid sequence of SEQ ID NO: 55; and (b) a VL comprising a VL CDR1 amino acid sequence of SEQ ID NO: 66, a VL CDR2 amino acid sequence of SEQ ID NO: 67, and a VL CDR3 amino acid sequence of SEQ ID NO: 68. In some embodiments, an anti-KIR3DL3 antibody or an antigen-binding fragment thereof described herein comprises: (a) a VH comprising a VH CDR1 amino acid sequence of SEQ ID NO: 56, a VH CDR2 amino acid sequence of SEQ ID NO: 57, and a VH CDR3 amino acid sequence of SEQ ID NO: 58; and (b) a VL comprising a VL CDR1 amino acid sequence of SEQ ID NO: 69, a VL CDR2 amino acid sequence of SEQ ID NO: 70, and a VL CDR3 amino acid sequence of SEQ ID NO: 71. In some embodiments, an anti-KIR3DL3 antibody or an antigen-binding fragment thereof described herein comprises: (a) a VH comprising a VH CDR1 amino acid sequence of SEQ ID NO: 59, a VH CDR2 amino acid sequence of SEQ ID NO: 60, and a VH CDR3 amino acid sequence of SEQ ID NO: 61; and (b) a VL comprising a VL CDR1 amino acid sequence of SEQ ID NO: 72, a VL CDR2 amino acid sequence of SEQ ID NO: 73, and a VL CDR3 amino acid sequence of SEQ ID NO: 74.

In some embodiments, an anti-KIR3DL3 antibody or an antigen-binding fragment thereof described herein comprises a VH comprising an amino acid sequence of SEQ ID NO: 62, or an amino acid sequence at least 85%, 90%, 95%, or 99% identical or higher to SEQ ID NO: 62. In some embodiments, an anti-KIR3DL3 antibody or an antigen-binding fragment thereof described herein comprises a VL comprising an amino acid sequence of SEQ ID NO: 75, or an amino acid sequence at least 85%, 90%, 95%, or 99% identical or higher to SEQ ID NO: 75. In some embodiments, an anti-KIR3DL3 antibody or an antigen-binding fragment thereof described herein comprises a VH comprising an amino acid sequence of SEQ ID NO: 62 and a VL comprising an amino acid sequence of SEQ ID NO: 75.

In some embodiments, an anti-KIR3DL3 antibody or an antigen-binding fragment thereof described herein comprises a heavy chain comprising an amino acid sequence of SEQ ID NO: 64, or an amino acid sequence at least 85%, 90%, 95%, or 99% identical or higher to SEQ ID NO: 64. In some embodiments, an anti-KIR3DL3 antibody or an antigen-binding fragment thereof described herein comprises a light chain comprising an amino acid sequence of SEQ ID NO: 77, or an amino acid sequence at least 85%, 90%, 95%, or 99% identical or higher to SEQ ID NO: 77. In some embodiments, an anti-KIR3DL3 antibody or an antigen-binding fragment thereof described herein comprises a heavy chain comprising an amino acid sequence of SEQ ID NO: 64, and a light chain comprising an amino acid sequence of SEQ ID NO: 77.

TABLE 1
Amino acid and nucleotide sequences
of exemplary anti-KIR3DL3 antibodies.

| | | SEQ ID |
|---|---|---|
| Clone | Sequences | NO |
| Hu26E | Heavy Chain | |
| 10z7p7 | HCDR1 (IMGT) | SEQ ID |
| (NPX267) | GYTFTSYN | NO: 1 |
| | HCDR2 (IMGT) | SEQ ID |
| | IYPGDAVT | NO: 2 |
| | HCDR3 (IMGT) | SEQ ID |
| | ARSGNYGNYEGFAY | NO: 3 |
| | HCDR1 (Kabat) | SEQ ID |
| | SYNIH | NO: 4 |
| | HCDR2 (Kabat) | SEQ ID |
| | YIYPGDAVTNYNQKFKG | NO: 5 |
| | HCDR3 (Kabat) | SEQ ID |
| | SGNYGNYEGFAY | NO: 6 |
| | HCDR1 (Combined Kabat/IMGT) | SEQ ID |
| | GYTFTSYNIH | NO: 7 |
| | HCDR2 (Combined Kabat/IMGT) | SEQ ID |
| | YIYPGDAVTNYNQKFKG | NO: 8 |
| | HCDR3 (Combined Kabat/IMGT) | SEQ ID |
| | SGNYGNYEGFAY | NO: 9 |
| | VH-Amino acid sequence | SEQ ID |
| | QAQLVQSGAEVKKPGASVKVSCKASGYTFTSYNIHWVRQAPG | NO: 10 |
| | QGLEWMGYIYPGDAVTNYNQKFKGRVTMTADTSTSTVYMELS | |
| | SLRSEDTAVYYCARSGNYGNYEGFAYWGQGTLVTVSS | |
| | VH-DNA sequence | SEQ ID |
| | CAGGCGCAGCTGGTGCAGAGCGGCGCGGAAGTGAAAAAACC | NO: 11 |
| | GGGCGCGAGCGTGAAAGTGAGCTGCAAAGCGAGCGGCTATA | |
| | CCTTTACCAGCTATAACATTCATTGGGTGCGCCAGGCGCCGG | |
| | GCCAGGGCCTGGAATGGATGGGCTATATTTATCCGGGCGAT | |
| | GCGGTGACCAACTATAACCAGAAATTTAAAGGCCGCGTGAC | |
| | CATGACCGCGGATACCAGCACCAGCACCGTGTATATGGAAC | |
| | TGAGCAGCCTGCGCAGCGAAGATACCGCGGTGTATTATTGC | |
| | GCGCGCAGCGGCAACTATGGCAACTATGAAGGCTTTGCGTA | |
| | TTGGGGCCAGGGCACCCTGGTGACCGTGAGCAGC | |
| | Heavy chain-Amino acid sequence | SEQ ID |
| | QAQLVQSGAEVKKPGASVKVSCKASGYTFTSYNIHWVRQAPG | NO: 12 |
| | QGLEWMGYIYPGDAVTNYNQKFKGRVTMTADTSTSTVYMELS | |
| | SLRSEDTAVYYCARSGNYGNYEGFAYWGQGTLVTVSSASTKG | |
| | PSVFPLAPCSRSTSESTAALGCLVKDYFPEPVTVSWNSGALTSG | |
| | VHTFPAVLQSSGLYSLSSVVTVPSSSLGTKTYTCNVDHKPSNTK | |
| | VDKRVESKYGPPCPPCPAPEFLGGPSVFLFPPKPKDTLMISRTPE | |
| | VTCVVVDVSQEDPEVQFNWYVDGVEVHNAKTKPREEQFNSTY | |
| | RVVSVLTVLHQDWLNGKEYKCKVSNKGLPSSIEKTISKAKGQP | |
| | REPQVYTLPPSQEEMTKNQVSLTCLVKGFYPSDIAVEWESNGQ | |
| | PENNYKTTPPVLDSDGSFFLYSRLTVDKSRWQEGNVFSCSVMH | |
| | EALHNHYTQKSLSLSLGK | |
| | Heavy chain-DNA sequence | SEQ ID |
| | CAGGCGCAGCTGGTGCAGAGCGGCGCGGAAGTGAAAAAACC | NO: 13 |
| | GGGCGCGAGCGTGAAAGTGAGCTGCAAAGCGAGCGGCTATA | |
| | CCTTTACCAGCTATAACATTCATTGGGTGCGCCAGGCGCCGG | |
| | GCCAGGGCCTGGAATGGATGGGCTATATTTATCCGGGCGAT | |
| | GCGGTGACCAACTATAACCAGAAATTTAAAGGCCGCGTGAC | |
| | CATGACCGCGGATACCAGCACCAGCACCGTGTATATGGAAC | |
| | TGAGCAGCCTGCGCAGCGAAGATACCGCGGTGTATTATTGC | |
| | GCGCGCAGCGGCAACTATGGCAACTATGAAGGCTTTGCGTA | |
| | TTGGGGCCAGGGCACCCTGGTGACCGTGAGCAGCGCGAGCA | |
| | CCAAAGGCCCGAGCGTGTTTCCGCTGGCGCCGTGCAGCCGC | |
| | AGCACCAGCGAAAGCACCGCGGCGCTGGGCTGCCTGGTGAA | |
| | AGATTATTTTCCGGAACCGGTGACCGTGAGCTGGAACAGCG | |
| | GCGCGCTGACCAGCGGCGTGCATACCTTTCCGGCGGTGCTGC | |
| | AGAGCAGCGGCCTGTATAGCCTGAGCAGCGTGGTGACCGTG | |
| | CCGAGCAGCAGCCTGGGCACCAAAACCTATACCTGCAACGT | |
| | GGATCATAAACCGAGCAACACCAAAGTGGATAAACGCGTGG | |
| | AAAGCAAATATGGCCCGCCGTGCCCGCCGTGCCCGGCGCCG | |
| | GAATTTCTGGGCGGCCCGAGCGTGTTTCTGTTTCCGCCGAAA | |
| | CCGAAAGATACCCTGATGATTAGCCGCACCCCGGAAGTGAC | |
| | CTGCGTGGTGGTGGATGTGAGCCAGGAAGATCCGGAAGTGC | |
| | AGTTTAACTGGTATGTGGATGGCGTGGAAGTGCATAACGCG | |
| | AAAACCAAACCGCGCGAAGAACAGTTTAACAGCACCTATCG | |
| | CGTGGTGAGCGTGCTGACCGTGCTGCATCAGGATTGGCTGAA | |
| | CGGCAAAGAATATAAATGCAAAGTGAGCAACAAAGGCCTGC | |
| | CGAGCAGCATTGAAAAAACCATTAGCAAAGCGAAAGGCCAG | |
| | CCGCGCGAACCGCAGGTGTATACCCTGCCGCCGAGCCAGGA | |
| | AGAAATGACCAAAAACCAGGTGAGCCTGACCTGCCTGGTGA | |
| | AAGGCTTTTATCCGAGCGATATTGCGGTGGAATGGGAAAGC | |
| | AACGGCCAGCCGGAAAACAACTATAAAACCACCCCGCCGGT | |
| | GCTGGATAGCGATGGCAGCTTTTTTCTGTATAGCCGCCTGAC | |
| | CGTGGATAAAAGCCGCTGGCAGGAAGGCAACGTGTTTAGCT | |
| | GCAGCGTGATGCATGAAGCGCTGCATAACCATTATACCCAG | |
| | AAAAGCCTGAGCCTGAGCCTGGGCAAA | |
| | Light Chain | |
| | LCDR1 (IMGT) | SEQ ID |
| | QDTQSY | NO: 14 |
| | LCDR2 (IMGT) | SEQ ID |
| | RA | NO: 15 |
| | LCDR3 (IMGT) | SEQ ID |
| | LQYDEFPYT | NO: 16 |
| | LCDR1 (Kabat) | SEQ ID |
| | KASQDTQSYLS | NO: 17 |
| | LCDR2 (Kabat) | SEQ ID |
| | RANRLVD | NO: 18 |
| | LCDR3 (Kabat) | SEQ ID |
| | LQYDEFPYT | NO: 19 |
| | LCDR1 (Combined Kabat/IMGT) | SEQ ID |
| | KASQDTQSYLS | NO: 20 |
| | LCDR2 (Combined Kabat/IMGT) | SEQ ID |
| | RANRLVD | NO: 21 |
| | LCDR3 (Combined Kabat/IMGT) | SEQ ID |
| | LQYDEFPYT | NO: 22 |
| | VL-Amino acid sequence | SEQ ID |
| | DIQMTQSPSSLSASVGDRVTITCKASQDTQSYLSWFQQKPGKAP | NO: 23 |
| | KTLIYRANRLVDGVPSRFSGSGSGTDFTLTISSLQPEDFATYYCL | |
| | QYDEFPYTFGGGTKVEIK | |
| | VL-DNA sequence | SEQ ID |
| | GATATTCAGATGACCCAGAGCCCGAGCAGCCTGAGCGCGAG | NO: 24 |
| | CGTGGGCGATCGCGTGACCATTACCTGCAAAGCGAGCCAGG | |
| | ATACCCAGAGCTATCTGAGCTGGTTTCAGCAGAAACCGGGC | |
| | AAAGCGCCGAAAACCCTGATTTATCGCGCGAACCGCCTGGT | |
| | GGATGGCGTGCCGAGCCGCTTTAGCGGCAGCGGCAGCGGCA | |
| | CCGATTTTACCCTGACCATTAGCAGCCTGCAGCCGGAAGATT | |
| | TTGCGACCTATTATTGCCTGCAGTATGATGAATTTCCGTATA | |
| | CCTTTGGCGGCGGCACCAAAGTGGAAATTAAA | |
| | Light chain-Amino acid sequence | SEQ ID |
| | DIQMTQSPSSLSASVGDRVTITCKASQDTQSYLSWFQQKPGKAP | NO: 25 |
| | KTLIYRANRLVDGVPSRFSGSGSGTDFTLTISSLQPEDFATYYCL | |
| | QYDEFPYTFGGGTKVEIKRTVAAPSVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFNRGEC | |
| | Light chain-DNA sequence | SEQ ID |
| | GATATTCAGATGACCCAGAGCCCGAGCAGCCTGAGCGCGAG | NO: 26 |
| | CGTGGGCGATCGCGTGACCATTACCTGCAAAGCGAGCCAGG | |
| | ATACCCAGAGCTATCTGAGCTGGTTTCAGCAGAAACCGGGC | |
| | AAAGCGCCGAAAACCCTGATTTATCGCGCGAACCGCCTGGT | |
| | GGATGGCGTGCCGAGCCGCTTTAGCGGCAGCGGCAGCGGCA | |
| | CCGATTTTACCCTGACCATTAGCAGCCTGCAGCCGGAAGATT | |
| | TTGCGACCTATTATTGCCTGCAGTATGATGAATTTCCGTATA | |
| | CCTTTGGCGGCGGCACCAAAGTGGAAATTAAACGCACCGTG | |
| | GCGGCGCCGAGCGTGTTTATTTTTCCGCCGAGCGATGAACAG | |
| | CTGAAAAGCGGCACCGCGAGCGTGGTGTGCCTGCTGAACAA | |
| | CTTTTATCCGCGCGAAGCGAAAGTGCAGTGGAAAGTGGATA | |
| | ACGCGCTGCAGAGCGGCAACAGCCAGGAAAGCGTGACCGAA | |
| | CAGGATAGCAAAGATAGCACCTATAGCCTGAGCAGCACCCT | |
| | GACCCTGAGCAAAGCGGATTATGAAAAACATAAAGTGTATG | |
| | CGTGCGAAGTGACCCATCAGGGCCTGAGCAGCCCGGTGACC | |
| | AAAAGCTTTAACCGCGGCGAATGC | |
| Hu12A | Heavy Chain | |
| 10z4p7 | HCDR1 (IMGT) | SEQ ID |
| | GYTFTSYN | NO: 27 |
| | HCDR2 (IMGT) | SEQ ID |
| | IFPGDAGT | NO: 28 |
| | HCDR3 (IMGT) | SEQ ID |
| | ARSGYYGNYEGFAY | NO: 29 |
| | HCDR1 (Kabat) | SEQ ID |
| | SYNMH | NO: 30 |
| | HCDR2 (Kabat) | SEQ ID |
| | YIFPGDAGTNYNQKFKG | NO: 31 |
| | HCDR3 (Kabat) | SEQ ID |
| | SGYYGNYEGFAY | NO: 32 |
| | HCDR1 (Combined Kabat/IMGT) | SEQ ID |
| | GYTFTSYNMH | NO: 33 |
| | HCDR2 (Combined Kabat/IMGT) | SEQ ID |
| | YIFPGDAGTNYNQKFKG | NO: 34 |
| | HCDR3 (Combined Kabat/IMGT) | SEQ ID |
| | SGYYGNYEGFAY | NO: 35 |
| | VH-Amino acid sequence | SEQ ID |
| | QVQLVQSGAEVKKPGASVKVSCKASGYTFTSYNMHWVRQAP | NO: 36 |
| | GQGLEWMGYIFPGDAGTNYNQKFKGRVTMTADTSTSTVYMEL | |
| | SSLRSEDTAVYYCARSGYYGNYEGFAYWGQGTLVTVSS | |
| | VH-DNA sequence | SEQ ID |
| | CAGGTGCAGCTGGTGCAGAGCGGCGCGGAAGTGAAAAAACC | NO: 37 |
| | GGGCGCGAGCGTGAAAGTGAGCTGCAAAGCGAGCGGCTATA | |
| | CCTTTACCAGCTATAACATGCATTGGGTGCGCCAGGCGCCGG | |
| | GCCAGGGCCTGGAATGGATGGGCTATATTTTTCCGGGCGATG | |
| | CGGGCACCAACTATAACCAGAAATTTAAAGGCCGCGTGACC | |
| | ATGACCGCGGATACCAGCACCAGCACCGTGTATATGGAACT | |
| | GAGCAGCCTGCGCAGCGAAGATACCGCGGTGTATTATTGCG | |
| | CGCGCAGCGGCTATTATGGCAACTATGAAGGCTTTGCGTATT | |
| | GGGGCCAGGGCACCCTGGTGACCGTGAGCAGC | |
| | Heavy chain-Amino acid sequence | SEQ ID |
| | QVQLVQSGAEVKKPGASVKVSCKASGYTFTSYNMHWVRQAP | NO: 38 |
| | GQGLEWMGYIFPGDAGTNYNQKFKGRVTMTADTSTSTVYMEL | |
| | SSLRSEDTAVYYCARSGYYGNYEGFAYWGQGTLVTVSSASTK | |
| | GPSVFPLAPCSRSTSESTAALGCLVKDYFPEPVTVSWNSGALTS | |
| | GVHTFPAVLQSSGLYSLSSVVTVPSSSLGTKTYTCNVDHKPSNT | |
| | KVDKRVESKYGPPCPPCPAPEFLGGPSVFLFPPKPKDTLMISRTP | |
| | EVTCVVVDVSQEDPEVQFNWYVDGVEVHNAKTKPREEQFNST | |
| | YRVVSVLTVLHQDWLNGKEYKCKVSNKGLPSSIEKTISKAKGQ | |
| | PREPQVYTLPPSQEEMTKNQVSLTCLVKGFYPSDIAVEWESNG | |
| | QPENNYKTTPPVLDSDGSFFLYSRLTVDKSRWQEGNVFSCSVM | |
| | HEALHNHYTQKSLSLSLGK | |
| | Heavy chain-DNA sequence | SEQ ID |
| | CAGGTGCAGCTGGTGCAGAGCGGCGCGGAAGTGAAAAAACC | NO: 39 |
| | GGGCGCGAGCGTGAAAGTGAGCTGCAAAGCGAGCGGCTATA | |
| | CCTTTACCAGCTATAACATGCATTGGGTGCGCCAGGCGCCGG | |
| | GCCAGGGCCTGGAATGGATGGGCTATATTTTTCCGGGCGATG | |
| | CGGGCACCAACTATAACCAGAAATTTAAAGGCCGCGTGACC | |
| | ATGACCGCGGATACCAGCACCAGCACCGTGTATATGGAACT | |
| | GAGCAGCCTGCGCAGCGAAGATACCGCGGTGTATTATTGCG | |
| | CGCGCAGCGGCTATTATGGCAACTATGAAGGCTTTGCGTATT | |
| | GGGGCCAGGGCACCCTGGTGACCGTGAGCAGCGCGAGCACC | |
| | AAAGGCCCGAGCGTGTTTCCGCTGGCGCCGTGCAGCCGCAG | |
| | CACCAGCGAAAGCACCGCGGCGCTGGGCTGCCTGGTGAAAG | |
| | ATTATTTTCCGGAACCGGTGACCGTGAGCTGGAACAGCGGC | |
| | GCGCTGACCAGCGGCGTGCATACCTTTCCGGCGGTGCTGCAG | |
| | AGCAGCGGCCTGTATAGCCTGAGCAGCGTGGTGACCGTGCC | |
| | GAGCAGCAGCCTGGGCACCAAAACCTATACCTGCAACGTGG | |
| | ATCATAAACCGAGCAACACCAAAGTGGATAAACGCGTGGAA | |
| | AGCAAATATGGCCCGCCGTGCCCGCCGTGCCCGGCGCCGGA | |
| | ATTTCTGGGCGGCCCGAGCGTGTTTCTGTTTCCGCCGAAACC | |
| | GAAAGATACCCTGATGATTAGCCGCACCCCGGAAGTGACCT | |
| | GCGTGGTGGTGGATGTGAGCCAGGAAGATCCGGAAGTGCAG | |
| | TTTAACTGGTATGTGGATGGCGTGGAAGTGCATAACGCGAA | |
| | AACCAAACCGCGCGAAGAACAGTTTAACAGCACCTATCGCG | |
| | TGGTGAGCGTGCTGACCGTGCTGCATCAGGATTGGCTGAACG | |
| | GCAAAGAATATAAATGCAAAGTGAGCAACAAAGGCCTGCCG | |
| | AGCAGCATTGAAAAAACCATTAGCAAAGCGAAAGGCCAGCC | |
| | GCGCGAACCGCAGGTGTATACCCTGCCGCCGAGCCAGGAAG | |
| | AAATGACCAAAAACCAGGTGAGCCTGACCTGCCTGGTGAAA | |
| | GGCTTTTATCCGAGCGATATTGCGGTGGAATGGGAAAGCAA | |
| | CGGCCAGCCGGAAAACAACTATAAAACCACCCCGCCGGTGC | |
| | TGGATAGCGATGGCAGCTTTTTTCTGTATAGCCGCCTGACCG | |
| | TGGATAAAAGCCGCTGGCAGGAAGGCAACGTGTTTAGCTGC | |
| | AGCGTGATGCATGAAGCGCTGCATAACCATTATACCCAGAA | |
| | AAGCCTGAGCCTGAGCCTGGGCAAA | |
| | Light Chain | |
| | LCDR1 (IMGT) | SEQ ID |
| | QDIQSY | NO: 40 |
| | LCDR2 (IMGT) | SEQ ID |
| | RA | NO: 41 |
| | LCDR3 (IMGT) | SEQ ID |
| | LHYAEFPYT | NO: 42 |
| | LCDR1 (Kabat) | SEQ ID |
| | KASQDIQSYLS | NO: 43 |
| | LCDR2 (Kabat) | SEQ ID |
| | RANRLVD | NO: 44 |
| | LCDR3 (Kabat) | SEQ ID |
| | LHYAEFPYT | NO: 45 |
| | LCDR1 (Combined Kabat/IMGT) | SEQ ID |
| | KASQDIQSYLS | NO: 46 |
| | LCDR2 (Combined Kabat/IMGT) | SEQ ID |
| | RANRLVD | NO: 47 |
| | LCDR3 (Combined Kabat/IMGT) | SEQ ID |
| | LHYAEFPYT | NO: 48 |
| | VL-Amino acid sequence | SEQ ID |
| | DIQMTQSPSSLSASVGDRVTITCKASQDIQSYLSWFQQKPGKAP | NO: 49 |
| | KTLIYRANRLVDGVPSRFSGSGSGTDFTLTISSLQPEDFATYYCL | |
| | HYAEFPYTFGGGTKVEIK | |
| | VL-DNA sequence | SEQ ID |
| | GATATTCAGATGACCCAGAGCCCGAGCAGCCTGAGCGCGAG | NO: 50 |
| | CGTGGGCGATCGCGTGACCATTACCTGCAAAGCGAGCCAGG | |
| | ATATTCAGAGCTATCTGAGCTGGTTTCAGCAGAAACCGGGCA | |
| | AAGCGCCGAAAACCCTGATTTATCGCGCGAACCGCCTGGTG | |
| | GATGGCGTGCCGAGCCGCTTTAGCGGCAGCGGCAGCGGCAC | |
| | CGATTTTACCCTGACCATTAGCAGCCTGCAGCCGGAAGATTT | |
| | TGCGACCTATTATTGCCTGCATTATGCGGAATTTCCGTATAC | |
| | CTTTGGCGGCGGCACCAAAGTGGAAATTAAA | |
| | Light chain-Amino acid sequence | SEQ ID |
| | DIQMTQSPSSLSASVGDRVTITCKASQDIQSYLSWFQQKPGKAP | NO: 51 |
| | KTLIYRANRLVDGVPSRFSGSGSGTDFTLTISSLQPEDFATYYCL | |
| | HYAEFPYTFGGGTKVEIKRTVAAPSVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFNRGEC | |
| | Light chain-DNA sequence | SEQ ID |
| | GATATTCAGATGACCCAGAGCCCGAGCAGCCTGAGCGCGAG | NO: 52 |
| | CGTGGGCGATCGCGTGACCATTACCTGCAAAGCGAGCCAGG | |
| | ATATTCAGAGCTATCTGAGCTGGTTTCAGCAGAAACCGGGCA | |
| | AAGCGCCGAAAACCCTGATTTATCGCGCGAACCGCCTGGTG | |
| | GATGGCGTGCCGAGCCGCTTTAGCGGCAGCGGCAGCGGCAC | |
| | CGATTTTACCCTGACCATTAGCAGCCTGCAGCCGGAAGATTT | |
| | TGCGACCTATTATTGCCTGCATTATGCGGAATTTCCGTATAC | |
| | CTTTGGCGGCGGCACCAAAGTGGAAATTAAACGCACCGTGG | |
| | CGGCGCCGAGCGTGTTTATTTTTCCGCCGAGCGATGAACAGC | |
| | TGAAAAGCGGCACCGCGAGCGTGGTGTGCCTGCTGAACAAC | |
| | TTTTATCCGCGCGAAGCGAAAGTGCAGTGGAAAGTGGATAA | |
| | CGCGCTGCAGAGCGGCAACAGCCAGGAAAGCGTGACCGAAC | |
| | AGGATAGCAAAGATAGCACCTATAGCCTGAGCAGCACCCTG | |
| | ACCCTGAGCAAAGCGGATTATGAAAAACATAAAGTGTATGC | |
| | GTGCGAAGTGACCCATCAGGGCCTGAGCAGCCCGGTGACCA | |
| | AAAGCTTTAACCGCGGCGAATGC | |
| Hu1G7 | Heavy Chain | |
| z9 | HCDR1 (IMGT) | SEQ ID |
| | GFTFSNYW | NO: 53 |
| | HCDR2 (IMGT) | SEQ ID |
| | IRLKSNNYAT | NO: 54 |
| | HCDR3 (IMGT) | SEQ ID |
| | TPFYGSSFPY | NO: 55 |
| | HCDR1 (Kabat) | SEQ ID |
| | NYWMN | NO: 56 |
| | HCDR2 (Kabat) | SEQ ID |
| | EIRLKSNNYATHYAESVKG | NO: 57 |
| | HCDR3 (Kabat) | SEQ ID |
| | FYGSSFPY | NO: 58 |
| | HCDR1 (Combined Kabat/IMGT) | SEQ ID |
| | GFTFSNYWMN | NO: 59 |
| | HCDR2 (Combined Kabat/IMGT) | SEQ ID |
| | EIRLKSNNYATHYAESVKG | NO: 60 |
| | HCDR3 (Combined Kabat/IMGT) | SEQ ID |
| | FYGSSFPY | NO: 61 |
| | VH-Amino acid sequence | SEQ ID |
| | ELQLVESGGGLVQPGGSLRLSCAASGFTFSNYWMNWVRQAPG | NO: 62 |
| | KGLEWVAEIRLKSNNYATHYAESVKGRFTISRDNAKNSLYLQM | |
| | NSLRAEDTAVYYCTPFYGSSFPYWGQGTLVTVSS | |
| | VH-DNA sequence | SEQ ID |
| | GAACTGCAGCTGGTGGAAAGCGGCGGCGGCCTGGTGCAGCC | NO: 63 |
| | GGGCGGCAGCCTGCGCCTGAGCTGCGCGGCGAGCGGCTTTA | |
| | CCTTTAGCAACTATTGGATGAACTGGGTGCGCCAGGCGCCGG | |
| | GCAAAGGCCTGGAATGGGTGGCGGAAATTCGCCTGAAAAGC | |
| | AACAACTATGCGACCCATTATGCGGAAAGCGTGAAAGGCCG | |
| | CTTTACCATTAGCCGCGATAACGCGAAAAACAGCCTGTATCT | |
| | GCAGATGAACAGCCTGCGCGCGGAAGATACCGCGGTGTATT | |
| | ATTGCACCCCGTTTTATGGCAGCAGCTTTCCGTATTGGGGCC | |
| | AGGGCACCCTGGTGACCGTGAGCAGC | |
| | Heavy chain-Amino acid sequence | SEQ ID |
| | ELQLVESGGGLVQPGGSLRLSCAASGFTFSNYWMNWVRQAPG | NO: 64 |
| | KGLEWVAEIRLKSNNYATHYAESVKGRFTISRDNAKNSLYLQM | |
| | NSLRAEDTAVYYCTPFYGSSFPYWGQGTLVTVSSASTKGPSVFP | |
| | LAPCSRSTSESTAALGCLVKDYFPEPVTVSWNSGALTSGVHTFP | |
| | AVLQSSGLYSLSSVVTVPSSSLGTKTYTCNVDHKPSNTKVDKR | |
| | VESKYGPPCPPCPAPEFLGGPSVFLFPPKPKDTLMISRTPEVTCV | |
| | VVDVSQEDPEVQFNWYVDGVEVHNAKTKPREEQFNSTYRVVS | |
| | VLTVLHQDWLNGKEYKCKVSNKGLPSSIEKTISKAKGQPREPQ | |
| | VYTLPPSQEEMTKNQVSLTCLVKGFYPSDIAVEWESNGQPENN | |
| | YKTTPPVLDSDGSFFLYSRLTVDKSRWQEGNVFSCSVMHEALH | |
| | NHYTQKSLSLSLGK | |
| | Heavy chain-DNA sequence | SEQ ID |
| | GAACTGCAGCTGGTGGAAAGCGGCGGCGGCCTGGTGCAGCC | NO: 65 |
| | GGGCGGCAGCCTGCGCCTGAGCTGCGCGGCGAGCGGCTTTA | |
| | CCTTTAGCAACTATTGGATGAACTGGGTGCGCCAGGCGCCGG | |
| | GCAAAGGCCTGGAATGGGTGGCGGAAATTCGCCTGAAAAGC | |
| | AACAACTATGCGACCCATTATGCGGAAAGCGTGAAAGGCCG | |
| | CTTTACCATTAGCCGCGATAACGCGAAAAACAGCCTGTATCT | |
| | GCAGATGAACAGCCTGCGCGCGGAAGATACCGCGGTGTATT | |
| | ATTGCACCCCGTTTTATGGCAGCAGCTTTCCGTATTGGGGCC | |
| | AGGGCACCCTGGTGACCGTGAGCAGCGCGAGCACCAAAGGC | |
| | CCGAGCGTGTTTCCGCTGGCGCCGTGCAGCCGCAGCACCAG | |
| | CGAAAGCACCGCGGCGCTGGGCTGCCTGGTGAAAGATTATT | |
| | TTCCGGAACCGGTGACCGTGAGCTGGAACAGCGGCGCGCTG | |
| | ACCAGCGGCGTGCATACCTTTCCGGCGGTGCTGCAGAGCAG | |
| | CGGCCTGTATAGCCTGAGCAGCGTGGTGACCGTGCCGAGCA | |
| | GCAGCCTGGGCACCAAAACCTATACCTGCAACGTGGATCAT | |
| | AAACCGAGCAACACCAAAGTGGATAAACGCGTGGAAAGCA | |
| | AATATGGCCCGCCGTGCCCGCCGTGCCCGGCGCCGGAATTTC | |
| | TGGGCGGCCCGAGCGTGTTTCTGTTTCCGCCGAAACCGAAAG | |
| | ATACCCTGATGATTAGCCGCACCCCGGAAGTGACCTGCGTGG | |
| | TGGTGGATGTGAGCCAGGAAGATCCGGAAGTGCAGTTTAAC | |
| | TGGTATGTGGATGGCGTGGAAGTGCATAACGCGAAAACCAA | |
| | ACCGCGCGAAGAACAGTTTAACAGCACCTATCGCGTGGTGA | |
| | GCGTGCTGACCGTGCTGCATCAGGATTGGCTGAACGGCAAA | |
| | GAATATAAATGCAAAGTGAGCAACAAAGGCCTGCCGAGCAG | |
| | CATTGAAAAAACCATTAGCAAAGCGAAAGGCCAGCCGCGCG | |
| | AACCGCAGGTGTATACCCTGCCGCCGAGCCAGGAAGAAATG | |
| | ACCAAAAACCAGGTGAGCCTGACCTGCCTGGTGAAAGGCTT | |
| | TTATCCGAGCGATATTGCGGTGGAATGGGAAAGCAACGGCC | |
| | AGCCGGAAAACAACTATAAAACCACCCCGCCGGTGCTGGAT | |
| | AGCGATGGCAGCTTTTTTCTGTATAGCCGCCTGACCGTGGAT | |
| | AAAAGCCGCTGGCAGGAAGGCAACGTGTTTAGCTGCAGCGT | |
| | GATGCATGAAGCGCTGCATAACCATTATACCCAGAAAAGCC | |
| | TGAGCCTGAGCCTGGGCAAA | |
| | Light Chain | |
| | LCDR1 (IMGT) | SEQ ID |
| | QDISNY | NO: 66 |
| | LCDR2 (IMGT) | SEQ ID |
| | YT | NO: 67 |
| | LCDR3 (IMGT) | SEQ ID |
| | QQGNALPFT | NO: 68 |
| | LCDR1 (Kabat) | SEQ ID |
| | RASQDISNYLN | NO: 69 |
| | LCDR2 (Kabat) | SEQ ID |
| | YTSILQL | NO: 70 |
| | LCDR3 (Kabat) | SEQ ID |
| | QQGNALPFT | NO: 71 |
| | LCDR1 (Combined Kabat/IMGT) | SEQ ID |
| | RASQDISNYLN | NO: 72 |
| | LCDR2 (Combined Kabat/IMGT) | SEQ ID |
| | YTSILQL | NO: 73 |
| | LCDR3 (Combined Kabat/IMGT) | SEQ ID |
| | QQGNALPFT | NO: 74 |
| | VL-Amino acid sequence | SEQ ID |
| | DIQMTQSPSSLSASVGDRVTITCRASQDISNYLNWYQQKPGKVP | NO: 75 |
| | KLLIYYTSILQLGVPSRFSGSGSGTDYTLTISSLQPEDVATYYCQ | |
| | QGNALPFTFGGGTKVEIK | |
| | VL-DNA sequence | SEQ ID |
| | GATATTCAGATGACCCAGAGCCCGAGCAGCCTGAGCGCGAG | NO: 76 |
| | CGTGGGCGATCGCGTGACCATTACCTGCCGCGCGAGCCAGG | |
| | ATATTAGCAACTATCTGAACTGGTATCAGCAGAAACCGGGC | |
| | AAAGTGCCGAAACTGCTGATTTATTATACCAGCATTCTGCAG | |
| | CTGGGCGTGCCGAGCCGCTTTAGCGGCAGCGGCAGCGGCAC | |
| | CGATTATACCCTGACCATTAGCAGCCTGCAGCCGGAAGATGT | |
| | GGCGACCTATTATTGCCAGCAGGGCAACGCGCTGCCGTTTAC | |
| | CTTTGGCGGCGGCACCAAAGTGGAAATTAAA | |
| | Light chain-Amino acid sequence | SEQ ID |
| | DIQMTQSPSSLSASVGDRVTITCRASQDISNYLNWYQQKPGKVP | NO: 77 |
| | KLLIYYTSILQLGVPSRFSGSGSGTDYTLTISSLQPEDVATYYCQ | |
| | QGNALPFTFGGGTKVEIKRTVAAPSVFIFPPSDEQLKSGTASVV | |
| | CLLNNFYPREAKVQWKVDNALQSGNSQESVTEQDSKDSTYSLS | |
| | STLTLSKADYEKHKVYACEVTHQGLSSPVTKSFNRGEC | |
| | Light chain-DNA sequence | SEQ ID |
| | GATATTCAGATGACCCAGAGCCCGAGCAGCCTGAGCGCGAG | NO: 78 |
| | CGTGGGCGATCGCGTGACCATTACCTGCCGCGCGAGCCAGG | |
| | ATATTAGCAACTATCTGAACTGGTATCAGCAGAAACCGGGC | |
| | AAAGTGCCGAAACTGCTGATTTATTATACCAGCATTCTGCAG | |
| | CTGGGCGTGCCGAGCCGCTTTAGCGGCAGCGGCAGCGGCAC | |
| | CGATTATACCCTGACCATTAGCAGCCTGCAGCCGGAAGATGT | |
| | GGCGACCTATTATTGCCAGCAGGGCAACGCGCTGCCGTTTAC | |
| | CTTTGGCGGCGGCACCAAAGTGGAAATTAAACGCACCGTGG | |
| | CGGCGCCGAGCGTGTTTATTTTTCCGCCGAGCGATGAACAGC | |
| | TGAAAAGCGGCACCGCGAGCGTGGTGTGCCTGCTGAACAAC | |
| | TTTTATCCGCGCGAAGCGAAAGTGCAGTGGAAAGTGGATAA | |
| | CGCGCTGCAGAGCGGCAACAGCCAGGAAAGCGTGACCGAAC | |
| | AGGATAGCAAAGATAGCACCTATAGCCTGAGCAGCACCCTG | |
| | ACCCTGAGCAAAGCGGATTATGAAAAACATAAAGTGTATGC | |
| | GTGCGAAGTGACCCATCAGGGCCTGAGCAGCCCGGTGACCA | |
| | AAAGCTTTAACCGCGGCGAATGC | |

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibody Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, each of which his hereby incorporated by reference in its entirety).

By way of example, anti-KIR3DL3 antibodies suitable for detecting KIR3DL3 protein are known in the art and include, e.g., antibodies Cat #: FAB8919R, MAB8919, FAB8919G, FAB8919N, FAB8919S, FAB8919T, FAB8919U, and FAB8919V (R&D systems); antibody AP52374PU-N(Origene): antibody PA5-26178 (ThermoFisher Scientific); antibodies OAAB05761, OAAF08125, OAAN04122, OACA09134, OACA09135, OACD04988; and OASG01190 (Aviva Systems Biology).

### Antigen-Binding Fragments

The present disclosure, among other things, provides anti-KIR3DL3 antigen-binding fragments. As used herein, an “anti-KIR3DL3 antigen-binding fragment” comprises or is any protein or peptide-containing molecule comprising at least a portion of an immunoglobulin molecule containing at least one complementarity determining region (CDR) of a VH or a VL or a KIR3DL3 binding portion derived from any of the antibodies described herein. Antibody fragments can be obtained using conventional techniques known to those of skill in the art, and the fragments are screened for utility in the same manner as intact antibodies. Such functional antibody fragments can retain the ability to selectively bind with KIR3DL3.

Examples of anti-KIR3DL3 antigen-binding fragments described herein can include: (i) a Fab fragment, a monovalent fragment comprising VL, VH, CL, and CH1 domains: (ii) a F(ab′)2 fragment, a bivalent fragment comprising two Fab fragments linked by a disulfide bridge at a hinge region: (iii) a Fd fragment comprising VH and CH1 domains; (iv) a Fv fragment comprising VL and VH domains of a single arm of an antibody, (v) a diabody (dAb) fragment comprising a VH domain: (vi) a camelid or camelized variable domain: (vii) a scFv, a fusion protein of VH and VL regions: or (viii) a single domain antibody. In some embodiments, an anti-KIR3DL3 antigen-binding fragment thereof described herein comprises or is a heavy chain and a light chain (e.g., a half antibody).

### Methods of Making

The present disclosure, among other things, provides methods of making anti-KIR3DL3 antibodies or antigen-binding fragments thereof described herein. In some embodiments, an anti-KIR3DL3 antibody or an antigen-binding fragment thereof described herein is identified using a display technology, such as yeast display, phage display, or ribosome display. In some embodiments, an anti-KIR3DL3 antibody or an antigen-binding fragment thereof described herein is identified using a hybridoma library (e.g., a mammalian hybridoma library, e.g., a mouse hybridoma library), followed by supernatant screening.

Combinatorial methods for generating antibodies are known in the art (as described in, e.g., Ladner et al. U.S. Pat. No. 5,223,409; Kang et al. International Publication No. WO 92/18619: Dower et al. International Publication No. WO 91/17271; Winter et al. International Publication WO 92/20791: Markland et al. International Publication No. WO 92/15679: Breitling et al. International Publication WO 93/01288; McCafferty et al. International Publication No. WO 92/01047: Garrard et al. International Publication No. WO 92/09690; Ladner et al. International Publication No. WO 90/02809; Fuchs et al. (1991) Bio/Technology 9:1370-1372; Hay et al. (1992) Hum Antibody Hybridomas 3:81-85: Huse et al. (1989) Science 246:1275-1281: Griffths et al. (1993) EMBO J 12:725-734: Hawkins et al. (1992) J Mol Biol 226:889-896; Clackson et al. (1991) Nature 352:624-628: Gram et al. (1992) PNAS 89:3576-3580; Garrad et al. (1991) Bio/Technology 9:1373-1377: Hoogenboom et al. (1991) Nuc Acid Res 19:4133-4137; and Barbas et al. (1991) PNAS 88:7978-7982, each of which his hereby incorporated by reference in its entirety).

In some embodiments, an anti-KIR3DL3 antibody or antigen-binding fragment thereof described herein may be derived from other species. A humanized antibody is an antibody produced by recombinant DNA technology, in which some or all amino acids of a human immunoglobulin light chain or heavy chain that are not required for antigen binding (e.g., constant regions and/or framework regions of variable domains) are used to substitute for the corresponding amino acids from light chain or heavy chain of the cognate, nonhuman antibody. By way of example, a humanized version of a murine antibody to a given antigen has on both heavy and light chains: (1) constant regions of a human antibody; (2) FRs from the variable domains of a human antibody; and (3) CDRs from the murine antibody. Human FRs may be selected based on their highest sequence homology to mouse FR sequence. When necessary, one or more residues in human FRs can be changed to residues at corresponding positions in a murine antibody so as to preserve binding affinity of the humanized antibody to a target. This change is sometimes called “back mutation.” Similarly, forward mutations may be made to revert back to murine sequence for a desired reason, e.g. stability or affinity to a target. Humanized antibodies generally are less likely to elicit an immune response in humans as compared to chimeric human antibodies because the former contain considerably fewer non-human components.

Methods for humanizing non-human antibodies are well known in the art. Suitable methods for making humanized antibodies in accordance with the present disclosure are described in, e.g., Winter EP 0 239 400; Jones et al., Nature 321:522-525 (1986); Riechmann et al., Nature 332:323-327 (1988): Verhoeven et al., Science 239:1534-1536 (1988): Queen et al., Proc. Nat. Acad. Sci. USA 86:10029 (1989): U.S. Pat. No. 6,180,370; and Orlandi et al., Proc. Natl. Acad. Sd. USA 86:3833 (1989): the disclosures of each of which are incorporated herein by reference in their entireties. Generally, transplantation of non-human (e.g., murine) CDRs onto a human antibody is achieved as follows. cDNAs encoding VH and VL are isolated from a hybridoma, and nucleic acid sequences encoding VH and VL including CDRs are determined by sequencing. Nucleic acid sequences encoding CDRs are inserted into corresponding regions of a human antibody VH or VL coding sequences and attached to human constant region gene segments of a desired isotype (e.g., γ1 for CH and κ for CL). Humanized heavy and light chain genes are co-expressed in mammalian host cells (e.g., CHO or NSO cells) to produce soluble humanized antibody. To facilitate large-scale production of antibodies, it is often desirable to select for a high expressor using, for example, a DHFR gene or GS gene in the producer line.

In some embodiments, an anti-KIR3DL3 antibody or antigen-binding fragment thereof described herein comprises or is a human antibody. Completely human antibodies may be particularly desirable for therapeutic treatment of human subjects. Human antibodies can be made by a variety of methods known in the art including phage display methods described above using antibody libraries derived from human immunoglobulin sequences (see, e.g., U.S. Pat. Nos. 4,444,887 and 4,716,111; and PCT publications WO 98/46645, WO 98/60433, WO 98/24893, WO 98/16664, WO 96/34096, WO 96/33735, and WO 91/10741: each of which is incorporated herein by reference in its entirety). Techniques are also available for the preparation of human monoclonal antibodies in, e.g., Cole et al., Monoclonal Antibodies and Cancer Therapy, Alan R. Riss, (1985); and Boerner et al., J. Immunol., 147 (1): 86-95. (1991), each of which is incorporated herein by reference in its entirety

### Nucleic Acids

The present disclosure, among other things, provides nucleic acids encoding KIR3DL3 binding agents described herein (e.g., anti-KIR3DL3 antibodies or antigen-binding fragments thereof). The present disclosure includes nucleic acids encoding one or more heavy chains, VH domains, heavy chain FRs, heavy chain CDRs, heavy chain constant domains, light chains, VL domains, light chain FRs, light chain CDRs, light chain constant domains, or other immunoglobulin-like sequences, antibodies, or antigen-binding fragments thereof disclosed herein. Such nucleic acids may be present in a vector. Such nucleic acids may be present in the genome of a cell, e.g., a cell of a subject in need of treatment or a cell for production of an antibody, e.g., a mammalian cell for production of an anti-KIR3DL3 antibodies or antigen-binding fragments thereof described herein.

Nucleic acids encoding KIR3DL3 binding agents described herein (e.g., an anti-KIR3DL3 antibody or antigen-binding fragment thereof) may be modified to include codons that are optimized for expression in a particular cell type or organism. Codon optimized sequences are synthetic sequences, and preferably encode an identical polypeptide (or biologically active fragment of a full length polypeptide which has substantially the same activity as the full length polypeptide) encoded by a non-codon optimized parent polynucleotide. In some embodiments, a coding region of a nucleic acids encoding KIR3DL3 binding agents described herein, in whole or in part, may include an altered sequence to optimize codon usage for a particular cell type (e.g., a eukaryotic or prokaryotic cell). For example, a coding sequence for a humanized heavy (or light) chain variable region described herein may be optimized for expression in a bacterial cells. Alternatively, the coding sequence may be optimized for expression in a mammalian cell (e.g., a CHO cell). Such a sequence may be described as a codon-optimized sequence.

Nucleic acid constructs of the present disclosure may be inserted into an expression vector or viral vector by methods known to the art, and nucleic acids may be operably linked to an expression control sequence. A vector comprising any nucleic acids or fragments thereof described herein is further provided by the present disclosure. Any nucleic acids or fragments thereof described herein can be cloned into any suitable vector and can be used to transform or transfect any suitable host. Selection of vectors and methods to construct them are commonly known to persons of ordinary skill in the art (see, e.g., “Recombinant DNA Part D,” Methods in Enzymology, Vol. 153, Wu and Grossman, eds., Academic Press (1987)).

Conventionally used techniques including, for example, electrophoresis, calcium phosphate precipitation, DEAE-dextran transfection, or lipofection, may be used to introduce a foreign nucleic acid (e.g., DNA or RNA) into a prokaryotic or eukaryotic host cell. Desirably, a vector may include regulatory sequences, such as transcription and/or translation initiation and/or termination codons, which are specific to the type of host (e.g., bacterium, fungus, plant, or animal) into which a vector is to be introduced, as appropriate and taking into consideration whether a vector is DNA or RNA. In some embodiments, a vector comprises regulatory sequences that are specific to a genus of a host cell. In some embodiments, a vector comprises regulatory sequences that are specific to a species of a host.

In addition to a replication system and an inserted nucleic acid, a nucleic acid construct can include one or more marker genes, which allow for selection of transformed or transfected hosts. Exemplary marker genes include, e.g., biocide resistance (e.g., resistance to antibiotics or heavy metals) or complementation in an auxotrophic host to provide prototrophy.

An expression vector can comprise a native or nonnative promoter operably linked to an isolated or purified nucleic acid as described above. Selection of promoters, e.g., strong, weak, inducible, tissue-specific, and/or developmental-specific, is within the skill of one in the art. Similarly, combining a nucleic acid as described above with a promoter is also within the skill of one in the art.

Suitable vectors include those designed for propagation and expansion and/or for expression. For example, a cloning vector may be selected from the pUC series, the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). Bacteriophage vectors, such as ΔGT10, ΔGT11, ΔZapII (Stratagene), ΔEMBL4, and ΔNM1149, may be used. Examples of plant expression vectors that can be used include pBI110, pBI101.2, pBI101.3, pBI121, or pBIN19 (Clontech). Examples of animal expression vectors that can be used include pEUK-C1, pMAM, or pMAMneo (Clontech). The TOPO cloning system (Invitrogen, Carlsbad, Calif.) also can be used in accordance with the manufacturer's recommendations.

Additional sequences can be added to such cloning and/or expression sequences to optimize their function in cloning and/or expression, to aid in isolation of a nucleic acid encoding an KIR3DL3 binding agent described herein, or to improve introduction of a nucleic acid into a cell. Use of cloning vectors, expression vectors, adapters, and linkers is well known in the art (see, e.g., Sambrook et al., Molecular Cloning, a Laboratory Manual, 2d edition, Cold Spring Harbor Press, Cold Spring Harbor, N.Y. (1989); and Ausubel et al., Current Protocols in Molecular Biology, Greene Publishing Associates and John Wiley & Sons, New York, N.Y. (1994), each of which is hereby incorporated by reference in its entirety).

In some embodiments, nucleic acids and vectors of the present disclosure are isolated and/or purified. The present disclosure also provides a composition comprising an isolated or purified nucleic acid, optionally in the form of a vector. Isolated nucleic acids and vectors may be prepared using standard techniques known in the art including, for example, alkali/SDS treatment, CsCl binding, column chromatography, agarose gel electrophoresis, and/or other techniques well known in the art. The composition can comprise other components as described further herein.

Any method known to one skilled in the art for the insertion of nucleic acids into a vector may be used to construct expression vectors encoding an anti-human KIR3DL3 antibody or antigen-binding fragment thereof described herein under control of transcriptional and/or translational control signals. These methods may include in vitro recombinant DNA and synthetic techniques and in vivo recombination (see, e.g., Ausubel, supra: or Sambrook, supra).

### Antibodies That Bind to the Same Epitope

In some embodiments, anti-KIR3DL3 antibodies or antigen-binding fragments thereof described herein include antibodies and antibody fragments that bind to the same epitope as the KIR3DL3-binding antibodies shown in Table 1 described herein. Additional antibodies and antibody fragments can therefore be identified based on their ability to cross-compete (e.g., to competitively inhibit the binding of, in a statistically significant manner) with other antibodies described herein in KIR3DL3 binding assays. The ability of a test antibody to inhibit the binding of antibodies and antibody fragments described herein to a KIR3DL3 protein (e.g., human KIR3DL3) demonstrates that the test antibody can compete with that antibody or antibody fragment for binding to KIR3DL3; such an antibody may, according to non-limiting theory, bind to the same or a related (e.g., a structurally similar or spatially proximal) epitope on the KIR3DL3 protein as the antibody or antibody fragment with which it competes. In some embodiment, an antibody that binds to the same epitope on KIR3DL3 as an anti-KIR3DL3 antibody or antigen-binding fragment thereof described herein is a human or humanized monoclonal antibody. Such human or humanized monoclonal antibodies can be prepared and isolated described herein.

### KIR3DL3 Gene Expression Modulators

Disclosed herein are methods, compositions, and formulations that include KIR3DL3 inhibitors that modulate gene expression, e.g., miRNA, shRNA, siRNA, CRISPR/Cas guide system, TALEN, or ZFN.

In some embodiments, a KIR3DL3 inhibitor comprises or is a gene expression modulator. Gene expression modulators can include RNAi molecules (e.g., double-stranded RNAs (dsRNAs), single-stranded RNAs (ssRNAs), micro RNAs (miRNAs), short interfering RNAs (siRNAs), short hairpin RNAs (shRNAs)) and triplex-forming oligonucleotides (TFOs)). Gene expression modulators may also include modified versions of any of the foregoing RNA molecules and, thus, include synthetic chemically modified RNAs.

In some embodiments, an RNAi can be a miRNA that reduces a level of KIR3DL3 in a cell (e.g., an NK cell or a T cell). In some embodiments, an RNAi can be a shRNA that reduces a level of KIR3DL3 in a cell (e.g., an NK cell or a T cell). In some embodiments, an RNAi can be a siRNA that reduces a level of KIR3DL3 in a cell (e.g., an NK cell or a T cell). Regulation of KIR3DL3 expression using miRNA and siRNA is described in, e.g., Nutalai et al., Genes (Basel). 2019; 10(8): 603, which is hereby incorporated by reference in its entirety.

Commercially available siRNAs and shRNAs for reducing KIR3DL3 expression include shRNA products #TF303684, TR303684, TG303684, TL303684, and TL303684V (HexaBiogen Groupe CliniSciences); shRNA products #sc-60892-SH and sc-60892-V (Santa Cruz. Biotechnology, Inc.); siRNA product #SR314516 (HexaBiogen Groupe CliniSciences); and siRNA product #sc-60892 (Santa Cruz Biotechnology, Inc.).

In some embodiments, a KIR3DL3 inhibitor comprises or is an endonuclease. Endonucleases can create breaks in double-stranded DNA at desired locations in a genome and use the host cell's mechanisms to repair the break using, for example, homologous recombination or nonhomologous end-joining. Classes of endonucleases that can be used for gene editing include, but are not limited to, clustered regularly interspaced short palindromic repeat(s) (CRISPR), transcription activator-like effector nucleases (TALENs) (see, e.g., U.S. Pat. No. 8,697,853: as well as U.S. Publication Nos. 20150118216, 20150079064, and 20140087426), zinc-finger nucleases (ZFNs) (see, e.g., U.S. Pat. Nos. 8,956,828; 8,921,112; 8,846,578; 8,569,253), meganucleases (see, e.g., U.S. Pat. Nos. 8,802,437, 8,445,251 and 8,338,157; and U.S. Publication Nos. 20130224863, 20110113509 and 20110033935), and homing endonucleases (see, e.g., U.S. Publication No. 20150166969; and U.S. Pat. No. 9,005,973).

In some embodiments, a KIR3DL3 inhibitor comprises or is a CRISPR/Cas system. The terms “CRISPR” or “CRISPR to KIR3DL3” or “CRISPR to inhibit KIR3DL3,” as used herein, refer to a set of clustered regularly interspaced short palindromic repeats or a system comprising such a set of repeats. “Cas”, as used herein, refers to a CRISPR-associated protein. A “CRISPR/Cas system” refers to a system derived from CRISPR and Cas, which can be used to silence or mutate a KIR3DL3 gene in an immune effector cell described herein, e.g., an NK cell or a T cell.

Commercially available CRISPR constructs for modulating KIR3DL3 expression include CRISPR products #KN224383, KN224383BN, KN224383RB, and KN224383LP (Origene Technologies): CRISPR products #K1151421, K1151401, K1151402, K1151403, K1151404, K1151405, K1151406, K1151407, K1151408, and K1151411 (Applied Biological Materials); and CRISPR products #sc-406227, sc-406227-KO-2, sc-406227-HDR-2, sc-406227-NIC, and sc-406227-NIC-2 (Santa Cruz Biotechnology).

In some embodiments, a KIR3DL3 inhibitor comprises or is a TALEN. The terms “TALEN” or “TALEN to KIR3DL3” or “TALEN to inhibit KIR3DL3.” as used herein, refer to a transcription activator-like effector nuclease, an artificial nuclease which can be used to edit a KIR3DL3 gene in an immune effector cell as described, e.g., an NK cell or a T cell.

In some embodiments, a KIR3DL3 inhibitor comprises or is a ZFN. The terms “ZFN” or “Zinc Finger Nuclease” or “ZFN to KIR3DL3” or “ZFN to inhibit KIR3DL3,” as used herein, refer to a zinc finger nuclease, an artificial nuclease which can be used to edit the KIR3DL3 gene in an immune effector cell described herein, e.g., an NK cell or a T cell.

### Demethylating Agents

In some embodiments, a KIR3DL3 inhibitor comprises or is a demethylating agent. A “demethylating agent,” as used herein, refers to a chemical substance that can inhibit methylation. In some embodiments, a demethylating agent comprises or is 5-aza-2-deoxycytidine (Aza), e.g., as described in Trundley et al, Immunogenetics (2006) 57:904-916, which is hereby incorporated by reference in its entirety. In some embodiments, a demethylating agent comprises or is 5-azacytidine. In some embodiments, a demethylating agent comprises or is 1-β-D-arabinofuranosil-5-azacytosine. In some embodiments, a demethylating agent comprises or is dihydro-5-azacytidine.

### Immune Cell Activating Agents

Disclosed herein are methods, compositions, and formulations that include at least one immune cell activating agent, e.g., cytokine agent, costimulatory antibody or antigen binding fragment thereof, polypeptides, glycoproteins, or exogenous cells.

As used herein, the term “immune cell activating agent” refers to an agent that activates an immune effector cell described herein (e.g., an NK cell or a T cell), thereby modifying the immune effector cell (e.g., by increasing proliferation and/or endogenous expression of an interleukin). An immune cell activating agent can include, but is not limited to, a cytokine agent (e.g., an interleukin, e.g., a cytokine (e.g., IL-2, IL-15, IL-12, IL-17, and/or IL-18)), an antibody or fragment thereof (e.g., a costimulatory antibody or fragment thereof), a polypeptide, a glycoprotein, an exogenous cell (e.g., an artificial antigen presenting cell), a nucleic acid, an antibiotic, an anti-inflammatory agent, a chimeric antigen receptor, a growth factor, an enzyme, a fusion protein, a synthetic molecule, an organic molecule (e.g., a small molecule), a carbohydrate, a lipid, a hormone, a microsome, a derivative or a variation thereof, and any combinations thereof. Immune cell activating agents may be endogenously expressed or exogenous to an immune effector cell described herein. An immune cell activating agent may bind any cell moiety, such as a receptor, an antigenic determinant, or other binding site present on an immune effector cell described herein. An immune cell activating agent may diffuse or be transported into a cell, where it may act intracellularly. An immune activating agent can be an antibody or antigen binding fragment thereof, or small molecule that decreases or blocks the inhibitory activity of one or more of a checkpoint protein (e.g., 4-1BB, CD40, CD28, OX40, GITR, PD-1, PD-L1, PD-L2, TIM-3, TGF-β, or LAG-3), an enzyme (e.g., CD39 or CD73), and/or a receptor (e.g., CTLA-4 or CD3).

In some embodiments, contacting an immune effector cell described herein with an immune cell activating agent described herein increases proliferation and/or increases endogenous expression of at least one interleukin (e.g., a cytokine) by immune effector cells. In some embodiments, contacting an immune effector cell described herein with at least one immune cell activating agent increases proliferation, e.g., relative to an immune effector cell not contacted with at least one immune cell activating agent. In some embodiments, contacting an immune effector cell described herein with at least one immune cell activating agent increases endogenous expression of at least one interleukin described herein, e.g., relative to an immune effector cell not contacted with at least one immune cell activating agent.

In some embodiments, an immune cell activating agent comprises or is a cytokine agent. In some embodiments, an immune cell activating agent comprises or is an interleukin. In some embodiments, a cytokine agent is or comprises IL-2, IL-15, IL-12, IL-17, IL-18, IL-21, IFNγ, or TNFα. In some embodiments, a cytokine agent is or comprises IL-2. In some embodiments, a cytokine agent is or comprises IL-15. In some embodiments, a cytokine agent is or comprises IL-12. In some embodiments, a cytokine agent is or comprises IL-17. In some embodiments, a cytokine agent is or comprises IL-18. In some embodiments, a cytokine agent is or comprises IL-21.

IL-2 is a member of a cytokine family including IL-4, IL-7, IL-9, IL-15 and IL-21, in which each member of this family has a four alpha helix bundle. IL-2 can be a T cell growth factor and is endogenously secreted by both CD4+ helper T cells and CD8+ T cells in vivo. IL-2 signals through the IL-2 receptor complex consisting of three chains, IL-2Rα (CD25), IL-2Rβ (CD122), and IL-2R (CD132). In some embodiments, IL-2 binds to IL-2Rα (CD25), IL-2Rβ (CD122), and/or IL-2R (CD132) to activate an immune effector cell or proliferation of an immune effector cell described herein. In some embodiments, IL-2 expands T cells (e.g., CD4+ helper T cells and/or CD8+ T cells). In some embodiments, IL-2 does not substantially expand Tregs. In some embodiments, IL-2 enhances cytotoxicity and/or expands NK cells.

In some embodiments, a cytokine agent comprises or is an inhibitor of a suppressor (e.g., an endogenous suppressor) of cytokine signaling. In some embodiments, a cytokine agent comprises or is a suppressor of cytokine signaling (SOCS) protein.

In some embodiments, the immune cell activating agent comprises or is a costimulatory antibody or antigen binding fragment thereof, or a costimulatory small molecule. In some embodiments, a costimulatory antibody or antigen binding fragment thereof binds to CD3, 4-1BB, CD40, CD28, OX40, GITR, CTLA-4, PD-1, PD-L1, PD-L2, TIM-3, TGF-β or LAG-3. In some embodiments, a costimulatory small molecule binds to CD3, 4-1BB, CD40, CD28, OX40, GITR, CTLA-4, PD-1, PD-L1, PD-L2, TIM-3, TGF-β, LAG-3, CD39, or CD73.

In some embodiments, a costimulatory antibody or antigen binding fragment thereof comprises or is an anti-CD3 antibody or fragment thereof. In some embodiments, an anti-CD3 antibody comprises or is OKT3 (also known as Muromonab-CD3 or Orthoclone OKT3). In some embodiments, an anti-CD3 antibody or fragment thereof binds to a T cell molecule, T3, associated with a T cell antigen receptor resulting in activation of T cells. In some embodiments, contacting an immune effector cell described herein with an anti-CD3 antibody or fragment thereof increases cytokine production by the immune effector cell, e.g., relative to an immune effector cell not contacted with an anti-CD3 antibody or fragment thereof. In some embodiments, contacting an immune effector cell described herein with an anti-CD3 antibody or fragment thereof enhances proliferation of an immune effector cell, e.g., relative to an immune effector cell not contacted with an anti-CD3 antibody or fragment thereof.

In some embodiments, a costimulatory antibody or antigen binding fragment thereof comprises or is an anti-4-1BB antibody or fragment thereof. In some embodiments, a costimulatory antibody or antigen binding fragment thereof comprises or is an anti-CD40 antibody or fragment thereof. In some embodiments, a costimulatory antibody or antigen binding fragment thereof comprises or is an anti-CD28 antibody or fragment thereof. In some embodiments, a costimulatory antibody or antigen binding fragment thereof comprises or is an anti-OX40 antibody or fragment thereof. In some embodiments, a costimulatory antibody or antigen binding fragment thereof comprises or is an anti-GITR antibody or fragment thereof. In some embodiments, a costimulatory antibody or antigen fragment thereof binding comprises or is an anti-CTLA-4 antibody or fragment thereof. In some embodiments, a costimulatory antibody or antigen binding fragment thereof comprises or is an anti-PD-1 antibody or fragment thereof. In some embodiments, a costimulatory antibody or antigen binding fragment thereof comprises or is an anti-PD-L1 antibody or fragment thereof. In some embodiments, a costimulatory antibody or antigen binding fragment thereof comprises or is an anti-PD-L2 antibody or fragment thereof. In some embodiments, a costimulatory antibody or antigen binding fragment thereof comprise or is an anti-TIM-3 antibody or fragment thereof. In some embodiments, a costimulatory antibody or antigen binding fragment thereof comprise or is an anti-TGF-β antibody or fragment thereof. In some embodiments, a costimulatory antibody or antigen binding fragment thereof comprise or is an anti-LAG-3 antibody or fragment thereof.

In some embodiments, the immune cell activating agent comprises or is a costimulatory polypeptide. In some embodiments, a costimulatory polypeptide comprises or is a soluble HHLA2 Fc fusion polypeptide. In some embodiments, a soluble HHLA2 Fc fusion polypeptide comprises an extracellular domain of human HHLA2. In some embodiments, a soluble HHLA2 Fc fusion polypeptide comprises a human IgG Fc region. In some embodiments, a soluble HHLA2 Fc fusion polypeptide comprises a human IgM Fc region. In some embodiments, an IgG is an IgG1. In some embodiments, a soluble HHLA2 Fc fusion polypeptide binds KIR3DL3 in an immune effector cell described herein. In some embodiments, a soluble HHLA2 Fc fusion polypeptide blocks an interaction between KIR3DL3 and HHLA2 in an immune effector cell described herein. A soluble HHLA2 Fc fusion polypeptide is described in WO2014/133728, which is hereby incorporated by reference in its entirety.

In some embodiments, an immune cell activating agent comprises or is a glycoprotein. In some embodiments, a costimulatory glycoprotein comprises or is a fibronectin protein or fragment thereof. Fibronectin is an endogenously expressed high-molecular weight (˜440 kDa) glycoprotein of the extracellular matrix known to bind to membrane-spanning integrins. In some embodiments, an immune cell activating agent comprises or is a recombinant human fibronectin protein or fragment thereof. In some embodiments, a recombinant human fibronectin fragment comprises a central cell-binding domain, a heparin-binding domain II, and a CS1-sequence. In some embodiments, a fibronectin protein or fragment thereof comprises or is RetroNectin® (Takara Bio Inc.). In some embodiments, contacting an immune effector cell described herein with a fibronectin protein or fragment thereof (e.g., RetroNectin®) increase proliferation of an immune effector cell described herein, e.g., relative to an immune effector cell not contacted with a fibronectin protein or fragment thereof.

In some embodiments, an immune cell activating agent comprises or is a costimulatory exogenous cell. In some embodiments, a costimulatory exogenous cell comprises or is an artificial antigen presenting cell (aAPC). In some embodiments, an aAPC comprises or is a K562-based aAPC. In some embodiments, contacting an immune effector cell described herein with an aAPC enhances cytokine production (e.g., IL-2 production) of an immune effector cell to enhance immune effector cell stimulation, e.g., relative to an immune effector cell not contacted with an aAPC. In some embodiments, contacting an immune effector cell described herein with an aAPC enhances proliferation of an immune effector cell, e.g., relative to an immune effector cell not contacted with an aAPC.

### Methods of Treatment

The present disclosure, among other things, provides methods of treating a disease, disorder or condition (e.g., a disease, disorder or condition described herein) in a subject comprising administering a pharmaceutical composition comprising at least one KIR3DL3 inhibitor described herein. In some embodiments, the at least one KIR3DL3 inhibitor is or comprises one or more anti-KIR3DL3 antibody or antigen-binding fragment thereof described herein.

In some embodiments, the disclosure provides at least one anti-KIR3DL3 antibody or antigen-binding fragment thereof described herein comprising amino acid sequences found in Table 1 for use as a medicament. In some embodiments, the disclosure provides at least one anti-KIR3DL3 antibody or antigen-binding fragment thereof described herein comprising amino acid sequences found in Table 1 for use in the treatment of a disease, disorder, or condition described herein. In some embodiments, the disclosure provides use of at least one anti-KIR3DL3 antibody or antigen-binding fragment thereof described herein comprising amino acid sequences found in Table 1 for the manufacture of a medicament for the treatment of a disease, disorder, or condition described herein.

The present disclosure, among other things, provides methods of treating a disease, disorder or condition (e.g., a disease, disorder or condition described herein) in a subject comprising administering a pharmaceutical composition comprising a population of modified immune effector cells described herein. In some embodiments, prior to administering, a population of immune effector cells described herein was contacted with at least one immune cell activating agent described herein and at least one KIR3DL3 inhibitor (e.g., an anti-KIR3DL3 antibody or antigen-binding fragment thereof) described herein, thereby forming a population of modified immune effector cells.

The present disclosure, among other things, also provides methods of treating a disease, disorder or condition (e.g., a disease, disorder or condition described herein) in a subject comprising delivering a pharmaceutical composition comprising a population of modified immune effector cells described herein and administering a pharmaceutical composition comprising at least one KIR3DL3 inhibitor described herein (e.g., an anti-KIR3DL3 antibody or antigen-binding fragment thereof described herein) to a subject. In some embodiments, prior to administering, a population of immune effector cells were contacted with at least one immune cell activating agent, thereby forming a population of modified immune effector cells.

In some embodiments, a therapeutically effective amount of at least one pharmaceutical composition described herein is administered to a subject having a disease, disorder, or condition. Pharmaceutical compositions described herein can be for use in the manufacture of a medicament for treating a disease, disorder, or condition in a subject or stimulating an immune response in a subject.

In some embodiments, a pharmaceutical composition comprising an anti-KIR3DL3 antibody or antigen-binding fragment thereof described herein is administered to a subject. In some embodiments, a pharmaceutical composition comprising a population of modified immune effector cells described herein is administered to a subject prior to administration of a pharmaceutical composition comprising at least one KIR3DL3 inhibitor described herein (e.g., an anti-KIR3DL3 antibody or antigen-binding fragment thereof described herein) to a subject. In some embodiments, a pharmaceutical composition comprising a population of modified immune effector cells described herein is administered to a subject after administration of a pharmaceutical composition comprising at least one KIR3DL3 inhibitor described herein (e.g., an anti-KIR3DL3 antibody or antigen-binding fragment thereof described herein) to a subject. In some embodiments, a pharmaceutical composition comprising a population of modified immune effector cells described herein is administered substantially simultaneously (e.g., co-administration by injection) with a pharmaceutical composition comprising at least one KIR3DL3 inhibitor described herein (e.g., an anti-KIR3DL3 antibody or antigen-binding fragment thereof described herein) to a subject. In some embodiments, a pharmaceutical composition comprising a population of modified immune effector cells and at least one KIR3DL3 inhibitor described herein (e.g., an anti-KIR3DL3 antibody or antigen-binding fragment thereof described herein) is administered to a subject (e.g., by injection).

In some embodiments, a pharmaceutical composition comprising a population of modified immune effector cells described herein is administered to a subject within less than about 3 hours of contacting the immune effector cells with at least one KIR3DL3 inhibitor described herein (e.g., an anti-KIR3DL3 antibody or antigen-binding fragment thereof described herein). In some embodiments, a pharmaceutical composition comprising a population of modified immune effector cells described herein is administered to a subject within less than about 1 minute, about 2 minutes, about 3 minutes, about 4 minutes, about 5 minutes, about 10 minutes, about 30 minutes, or about 45 minutes, about 1 hour, about 1.5 hours, about 2 hours, about 2.5 hours, or about 3 hours of contacting the immune effector cells with at least one KIR3DL3 inhibitor described herein (e.g., an anti-KIR3DL3 antibody or antigen-binding fragment thereof described herein).

A subject to be treated with methods described herein can be a mammal, e.g., a primate, e.g., a human (e.g., a patient having, or at risk of having, a disease, disorder or condition described herein). In some embodiments, immune effector cells (e.g., NK cells or T cells) may be autologous, allogeneic, or xenogeneic with respect to a subject. Pharmaceutical compositions described herein can be administered to a subject in accordance with a dosage regimen described herein, alone or in combination with one or more therapeutic agents, procedures, or modalities.

A method of treating (e.g., one or more of reducing, inhibiting, or delaying progression of) a cancer or a tumor in a subject with a pharmaceutical composition comprising immune cells described herein (e.g., NK cells or T cells) and/or a pharmaceutical composition comprising at least one KIR3DL3 inhibitor (e.g., an anti-KIR3DL3 antibody or antigen-binding fragment thereof described herein) is provided. A subject can have an adult or pediatric form of cancer. A cancer may be at an early, intermediate, or late stage, or a metastatic cancer. In some embodiments, a subject has a cancer that is resistant to a therapeutic agent, e.g., comprising a cytokine agent described herein.

A method of treating (e.g., one or more of reducing, inhibiting, or delaying progression of) a sign or symptom of cancer in a subject with a pharmaceutical composition comprising immune cells described herein (e.g., NK cells or T cells) and/or a pharmaceutical composition comprising at least one KIR3DL3 inhibitor (e.g., an anti-KIR3DL3 antibody or antigen-binding fragment thereof described herein) is provided. In some embodiments, pharmaceutical composition described herein are useful to delay the onset of, slow the progression of, or ameliorate one or more signs or symptoms of cancer. In some embodiments, a physiological sign or symptom of cancer comprises or is an increase in tumor volume, an increase in number of cancer cells, an increase in number of metastases, a decrease in life expectancy, an increase in cancer cell proliferation, and/or an increase in cancer cell survival. In some embodiments, a physical sign or symptom of cancer comprises or is a skin lesion (e.g., a lump or mole), weight loss, digestive problems, discomfort, fatigue, pain, trouble swallowing, cough, unusual bleeding and/or discharge, changes in bowel and/or bladder habits, and/or mental confusion.

A cancer can include, but is not limited to, a solid tumor, a hematological cancer (e.g., leukemia, lymphoma, or myeloma, e.g., multiple myeloma), or a metastatic lesion. Examples of solid tumors include malignancies, e.g., sarcomas and carcinomas, e.g., adenocarcinomas of the various organ systems, such as those affecting the lung, breast, ovarian, lymphoid, gastrointestinal (e.g., colon), anal, genitals and genitourinary tract (e.g., renal, urothelial, bladder cells, prostate), pharynx, CNS (e.g., brain, neural or glial cells), head and neck, skin (e.g., melanoma, e.g., a cutaneous melanoma), pancreas, and bones (e.g., a chordoma).

In some embodiments, a cancer is chosen from a lung cancer (e.g., a non-small cell lung cancer (NSCLC) (e.g., a non-small cell lung cancer (NSCLC) with squamous and/or non-squamous histology, or a NSCLC adenocarcinoma), or a small cell lung cancer (SCLC)), a skin cancer (e.g., a Merkel cell carcinoma or a melanoma (e.g., an advanced melanoma)), an ovarian cancer, a mesothelioma, a bladder cancer, a soft tissue sarcoma (e.g., a hemangiopericytoma (HPC)), a bone cancer (a bone sarcoma), a kidney cancer (e.g., a renal cancer (e.g., a renal cell carcinoma)), a liver cancer (e.g., a hepatocellular carcinoma), a cholangiocarcinoma, a sarcoma, a myelodysplastic syndrome (MDS), a prostate cancer, a breast cancer (e.g., a breast cancer that does not express one, two or all of estrogen receptor, progesterone receptor, or Her2/neu, e.g., a triple negative breast cancer), a colorectal cancer (e.g., a relapsed colorectal cancer or a metastatic colorectal cancer, e.g., a microsatellite unstable colorectal cancer, a microsatellite stable colorectal cancer, a mismatch repair proficient colorectal cancer, or a mismatch repair deficient colorectal cancer), a nasopharyngeal cancer, a duodenal cancer, an endometrial cancer, a pancreatic cancer, a head and neck cancer (e.g., head and neck squamous cell carcinoma (HNSCC)), an anal cancer, a gastro-esophageal cancer, a thyroid cancer (e.g., anaplastic thyroid carcinoma), a cervical cancer (e.g., a squamous cell carcinoma of the cervix), a neuroendocrine tumor (NET) (e.g., an atypical pulmonary carcinoid tumor), a lymphoproliferative disease (e.g., a post-transplant lymphoproliferative disease), a lymphoma (e.g., T-cell lymphoma, B-cell lymphoma, or a non-Hodgkin lymphoma), a myeloma (e.g., a multiple myeloma), or a leukemia (e.g., a myeloid leukemia or a lymphoid leukemia). In some embodiments, a subject has renal cell carcinoma.

In some embodiments, a cancer is a brain tumor, e.g., a glioblastoma, a gliosarcoma, or a recurrent brain tumor. In some embodiments, a cancer is a pancreatic cancer, e.g., an advanced pancreatic cancer. In some embodiments, a cancer is a skin cancer, e.g., a melanoma (e.g., a stage II-IV melanoma, an HLA-A2 positive melanoma, an unresectable melanoma, or a metastatic melanoma), or a Merkel cell carcinoma. In some embodiments, a cancer is a renal cancer, e.g., a renal cell carcinoma (RCC) (e.g., a metastatic renal cell carcinoma). In some embodiments, a cancer is a breast cancer, e.g., a metastatic breast carcinoma or a stage IV breast carcinoma, e.g., a triple negative breast cancer (TNBC). In some embodiments, a cancer is a virus-associated cancer. In some embodiments, a cancer is an anal canal cancer (e.g., a squamous cell carcinoma of the anal canal). In some embodiments, a cancer is a cervical cancer (e.g., a squamous cell carcinoma of the cervix). In some embodiments, a cancer is a gastric cancer (e.g., an Epstein Barr Virus (EBV) positive gastric cancer, or a gastric or gastro-esophageal junction carcinoma). In some embodiments, a cancer is a head and neck cancer (e.g., an HPV positive and negative squamous cell cancer of the head and neck (SCCHN)). In some embodiments, a cancer is a nasopharyngeal cancer (NPC). In some embodiments, a cancer is a colorectal cancer, e.g., a relapsed colorectal cancer, a metastatic colorectal cancer, e.g., a microsatellite unstable colorectal cancer, a microsatellite stable colorectal cancer, a mismatch repair proficient colorectal cancer, or a mismatch repair deficient colorectal cancer.

In some embodiments, a cancer is a hematological cancer. In some embodiments, a cancer is a leukemia, e.g., acute myeloid leukemia, chronic myeloid leukemia, acute lymphoblastic leukemia, chronic lymphocytic leukemia, chronic leukemia, or acute leukemia. In some embodiments, a cancer is a lymphoma, e.g., Hodgkin lymphoma (HL), non-Hodgkin's lymphoma, lymphocytic lymphoma, or diffuse large B cell lymphoma (DLBCL) (e.g., a relapsed or refractory HL or DLBCL). In some embodiments, a cancer is a myeloma, e.g., multiple myeloma.

Administration of pharmaceutical compositions described herein may be carried out in any convenient manner (e.g., injection, ingestion, transfusion, inhalation, implantation, or transplantation). In some embodiments, a pharmaceutical compositions described herein is administered by injection or infusion. Pharmaceutical compositions described herein may be administered to a patient transarterially, subcutaneously, intravenously, intradermally, intratumorally, intranodally, intramedullary, intramuscularly, or intraperitoneally. In some embodiments, a pharmaceutical composition described herein is administered parenterally (e.g., intravenously, subcutaneously, intraperitoneally, or intramuscularly). In some embodiments, a pharmaceutical composition described herein is administered by subcutaneous, intravenous, intramuscular, or intrasternal infusion or injection. In some embodiments, a pharmaceutical composition described herein is administered by intramuscular or subcutaneous injection. Pharmaceutical compositions described herein may be injected directly into a site of inflammation, a local disease site, a lymph node, an organ, a tumor, or site of infection in a subject.

A subject to be treated with methods described herein can be a mammal (e.g., a primate, mouse, humanized mouse, rat, non-human mammal, domestic animal, such as a dog, cat, cow, or horse), and is preferably a human (e.g., a patient having, or at risk of having, a disease, disorder or condition described herein). A subject can be an animal model of cancer, such as a xenograft animal model of a human-derived cancer. In some embodiments, a subject has not undergone treatment, such as chemotherapy, radiation therapy, targeted therapy, and/or immune checkpoint therapy. In another embodiment, a subject has undergone treatment, such as chemotherapy, radiation therapy, targeted therapy, and/or immune checkpoint therapy. In certain embodiments, a subject has had surgery to remove cancerous or precancerous tissue. In other embodiments, a cancerous tissue has not been removed, e.g., the cancerous tissue may be located in an inoperable region of the body, such as in a tissue that is essential for life or in a region where a surgical procedure would cause considerable risk of harm to a subject.

### Additional Agents

The present disclosure, among other things, provides one or more additional agents (e.g., 2, 3, 4, 5, or more additional agents) that can be administered with an anti-KIR3DL3 antibody or an antigen-binding fragment thereof described herein in a combination therapy. As used herein, an additional agent may be or comprise any known treatment for a particular disease, disorder, or condition (e.g., a cancer). For example, an additional agent can be or comprise one or more of: a chemotherapeutic agent, immune checkpoint inhibitor, a gene expression modulators, an immunomodulatory interleukin, an immunomodulatory chemokine, a hormone therapy, a cell-based therapy, a cancer vaccine, an epigenetic modifiers (e.g., histone deacetylase (HDAC) modifier, an immunomodulatory drug, immunomodulatory antibody, a nutritional supplement, treatment with hyperthermia, a photodynamic therapy, surgery, radiation, or a transplant.

In some embodiments, a chemotherapeutic agent comprises or is one or more anthracyclines, one or more cytoskeletal disruptors (e.g. microtubule targeting agents such as taxanes, maytansine, and analogs thereof), one or more epothilones, one or more histone deacetylase inhibitors (HDACs), one or more topoisomerase inhibitors (e.g., one or more of inhibitors of topoisomerase I or topoisomerase II), one or more kinase inhibitors, one or more nucleotide analogs or nucleotide precursor analogs, one or more peptide antibiotics, one or more platinum-based agents, one or more retinoids, one or more vinca alkaloids, or a combination thereof.

In some embodiments, a chemotherapeutic agent comprises or is one or more of: Actinomycin, All-trans retinoic acid, an Auristatin, Azacitidine, Azathioprine, Bleomycin, Bortezomib, Carboplatin, Capecitabine, Cisplatin, Chlorambucil, Cyclophosphamide, Curcumin, Cytarabine, Daunorubicin, Docetaxel, Doxifluridine, Doxorubicin, Epirubicin, Epothilone, Etoposide, Fludarabine, Fluorouracil, Gemcitabine, Hydroxyurea, Idarubicin, Imatinib, Irinotecan, Maytansine and/or analogs thereof (e.g. DM1), Mechlorethamine, Mercaptopurine, Methotrexate, Mitoxantrone, a Maytansinoid, Oxaliplatin, Paclitaxel, Pemetrexed, Rituxan, Teniposide, Tioguanine, Topotecan, Valrubicin, Vinblastine, Vincristine, Vindesine, or Vinorelbine. In some embodiments, a chemotherapeutic agent comprises or is an antibody-drug conjugate (ADC). In some embodiments, an ADC comprises or is hLL1-doxorubicin, hRS7-SN-38, hMN-14-SN-38, hLL2-SN-38, hA20-SN-38, hPAM4-SN-38, hLL1-SN-38, hRS7-Pro-2-P-Dox, hMN-14-Pro-2-P-Dox, hLL2-Pro-2-P-Dox, hA20-Pro-2-P-Dox, hPAM4-Pro-2-P-Dox, hLL1-Pro-2-P-Dox, P4/D10-doxorubicin, gemtuzumab ozogamicin, brentuximab vedotin, trastuzumab emtansine, inotuzumab ozogamicin, glembatumomab vedotin, SAR3419, SAR566658, BIIB015, BT062, SGN-75, SGN-CD19A, AMG-172, AMG-595, BAY-94-9343, ASG-5ME, ASG-22ME, ASG-16M8F, MDX-1203, MLN-0264, anti-PSMA ADC, RG-7450, RG-7458, RG-7593, RG-7596, RG-7598, RG-7599, RG-7600, RG-7636, ABT-414, IMGN-853, IMGN-529, vorsetuzumab mafodotin, lorvotuzumab mertansine, or a combination thereof.

In some embodiments, an immune checkpoint inhibitor comprises or is an agent that targets one or more of: CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, HHLA2, TMIDG2, KIR3DL3, and A2aR.

In some embodiments, gene expression modulators comprise or are one or more of an inhibitory nucleic acid, a CRISPR/Cas guide system, TALEN, or ZFN. Inhibitory nucleic acids can include RNAi molecules (e.g., double-stranded RNAs (dsRNAs), single-stranded RNAs (ssRNAs), micro RNAs (miRNAs), short interfering RNAs (siRNAs), short hairpin RNAs (shRNAs)) and triplex-forming oligonucleotides (TFOs)). Gene expression modulators may also include modified versions of any of the foregoing RNA molecules and, thus, include synthetic chemically modified RNAs. In some embodiments, a CRISPR/Cas guide system can comprise Cas9, Cas 12 (e.g., Cas12a), or Cas 13.

In some embodiments, immunomodulatory interleukins comprise or are one or more of: IL-2, IL-6, IL-7, IL-12, IL-17, or IL-23. In some embodiments, immunomodulatory chemokines comprise or are one or more of: CCL3, CCL26, and CXCL7.

In some embodiments, immunomodulatory drugs comprise or are immunocytostatic drugs, glucocorticoids, cytostatics, immunophilins and modulators thereof (e.g., rapamycin, a calcineurin inhibitor, tacrolimus, ciclosporin (cyclosporin), pimecrolimus, abetimus, gusperimus, ridaforolimus, everolimus, temsirolimus, or zotarolimus), hydrocortisone (cortisol), cortisone acetate, prednisone, prednisolone, methylprednisolone, dexamethasone, betamethasone, triamcinolone, beclometasone, fludrocortisone acetate, deoxy corticosterone acetate (doca) aldosterone, a non-glucocorticoid steroid, a pyrimidine synthesis inhibitor, leflunomide, teriflunomide, a folic acid analog, methotrexate, anti-thymocyte globulin, anti-lymphocyte globulin, thalidomide, lenalidomide, pentoxifylline, bupropion, curcumin, catechin, an opioid, an IMPDH inhibitor, mycophenolic acid, myriocin, fingolimod, an NF-xB inhibitor, raloxifene, drotrecogin alfa, denosumab, an NF-kB signaling cascade inhibitor, disulfiram, olmesartan, dithiocarbamate, a proteasome inhibitor, bortezomib, MG132, Prol, NPI-0052, curcumin, genistein, resveratrol, parthenolide, thalidomide, lenalidomide, flavopiridol, non-steroidal anti-inflammatory drugs (NSAIDs), arsenic trioxide, dehydroxymethylepoxyquinomycin (DHMEQ), 13C (indole-3-carbinol)/DIM (di-indolmethane) (13C/DIM), Bay 11-7082, luteolin, cell permeable peptide SN-50, IKBa.-super repressor overexpression, NFKB decoy oligodeoxynucleotide (ODN), or a derivative or analog of any of the foregoing.

In some embodiments, an immunomodulatory antibody comprises or is one or more of: antibodies that bind to CD40. Toll-like receptor (TLR), OX40, GITR, CD27, or 4-1BB, T-cell bispecific antibodies, an anti-IL-2 receptor antibody, an anti-CD3 antibody, OKT3 (muromonab), otelixizumab, teplizumab, visilizumab, an anti-CD4 antibody, clenoliximab, keliximab, zanolimumab, an anti-CD11 antibody, efalizumab, an anti-CD18 antibody, erlizumab, rovelizumab, an anti-CD20 antibody, afutuzumab, ocrelizumab, ofatumumab, pascolizumab, rituximab, an anti-CD23 antibody, lumiliximab, an anti-CD40) antibody, teneliximab, toralizumab, an anti-CD40L antibody, ruplizumab, an anti-CD62L antibody, aselizumab, an anti-CD80 antibody, galiximab, an anti-CD147 antibody, gavilimomab, a B-Lymphocyte stimulator (BLyS) inhibiting antibody, belimumab, an CTLA4-Ig fusion protein, abatacept, belatacept, an anti-CTLA4 antibody, ipilimumab, tremelimumab, an anti-cotaxin 1 antibody, bertilimumab, an anti-α4-integrin antibody, natalizumab, an anti-IL-6R antibody, tocilizumab, an anti-LFA-1 antibody, odulimomab, an anti-CD25 antibody, basiliximab, daclizumab, inolimomab, an anti-CD5 antibody, zolimomab, an anti-CD2 antibody, siplizumab, nerelimomab, faralimomab, atlizumab, atorolimumab, cedelizumab, dorlimomab aritox, dorlixizumab, fontolizumab, gantenerumab, gomiliximab, lebrilizumab, maslimomab, morolimumab, pexelizumab, reslizumab, rovelizumab, talizumab, telimomab aritox, vapaliximab, vepalimomab, aflibercept, alefacept, rilonacept, an IL-1 receptor antagonist, anakinra, an anti-IL-5 antibody, mepolizumab, an IgE inhibitor, omalizumab, an IL12 inhibitor, an IL23 inhibitor, or ustekinumab.

In some embodiments, a hormone therapy may be or comprise tamoxifen, raloxifene, leuprolide, bicaluatmide, granisetron, flutamide, or a combination thereof. In some embodiments, a cell-based therapy comprises or is chimeric antigen receptor T (CAR-T) cells, CAR-NK cells, TCR-transduced T cells, dendritic cells, tumor infiltrating lymphocytes (TILs), natural killer (NK) cells, Irradiated autologous or allogeneic tumor cells, or a combination thereof. In some embodiments, treatment with hyperthermia comprises or is local hyperthermia (e.g., external, intraluminal, or interstitial hyperthermia), regional hyperthermia (e.g., deep tissue hyperthermia, regional perfusion, or (continuous hyperthermic peritoneal perfusion), or whole-body hyperthermia. In some embodiments, a photodynamic therapy comprises or is administration of photosensitizers, such as hematoporphyrin and its derivatives, Verteporfin (BPD-MA), phthalocyanine, photosensitizer Pc4, demethoxy-hypocrellin A, 2BA-2-DMHA, or a combination thereof. In some embodiments, surgery comprises or is surgery to remove cancerous or precancerous tissue. In some embodiments, a transplant comprises or is a stem cell transplant or an organ transplant.

In some embodiments, a nutritional supplement comprises or is one or more of vitamin A, vitamin E, vitamin C, and the like (see, for example, U.S. Pat. Nos. 4,981,844 and 5,230,902 and PCT Publ. No. WO 2004/004483).

In some embodiments, an additional agent is administered prior to, substantially simultaneously with, or subsequent to administration of an anti-KIR3DL3 antibody or an antigen-binding fragment thereof described herein. In some embodiments, administration of an anti-KIR3DL3 antibody or an antigen-binding fragment thereof described herein and an additional agent results in an improvement in a disease, disorder, or condition (e.g., cancer) to an extent that is greater than an improvement produced by either the bispecific antibody molecule or antigen-binding fragment thereof described herein or the additional agent alone. The difference between the combined effect and the effect of each agent alone can be a statistically significant difference. In some embodiments, the combined effect can be a synergistic effect. In some embodiments, combined administration of an anti-KIR3DL3 antibody or an antigen-binding fragment thereof described herein and an additional agent allows administration of the additional agent at a reduced dose, a reduced number of doses, and/or a reduced frequency of dosage compared to a standard dosing regimen, e.g., an approved dosing regimen for the additional agent.

### Conjugates to Therapeutic Agents

The present disclosure, among other things, provides anti-KIR3DL3 antibodies or antigen-binding fragments thereof conjugated to one or more therapeutic agents. In some embodiments, a therapeutic agent comprises or is a cytotoxic agent, a drug, and/or a radioisotope. When conjugated to a cytotoxic agent, such conjugates can be referred to as “immunotoxins.” A cytotoxic agent includes any agent that is detrimental to (e.g., can kill) cells. Examples of cytotoxic agents include, but are not limited to, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs of any of the foregoing.

Anti-KIR3DL3 antibodies or antigen-binding fragments thereof described herein can be conjugated to one or more therapeutic agents (e.g., one or more drugs) including, but are not limited to, antimetabolites (e.g., methotrexate, 6-mercaptopurine, 6-thioguanine, cytarabine, and/or 5-fluorouracil decarbazine), topoisomerase I inhibitors (e.g., deruxtecan), alkylating agents (e.g., mechlorethamine, thiotepa chlorambucil, melphalan, carmustine (BSNU), lomustine (CCNU), cyclophosphamide, busulfan, dibromomannitol, streptozotocin, mitomycin C, and/or cis-dichlorodiamine platinum (II) (DDP) cisplatin), anthracyclines (e.g., daunorubicin (formerly daunomycin) and/or doxorubicin), antibiotics (e.g., dactinomycin, bleomycin, mithramycin, and/or anthramycin (AMC)), and anti-mitotic agents (e.g., vincristine and/or vinblastine). Anti-KIR3DL3 antibodies or antigen-binding fragments thereof described herein can be conjugated to one or more radioisotopes (e.g., radioactive iodine) to generate cytotoxic radiopharmaceuticals for treating diseases, disorders, or conditions described herein, such as cancers described herein.

### Pharmaceutical Compositions

The present disclosure, among other things, provides pharmaceutical compositions comprising a population of immune effector cells described herein (e.g., NK cells or T cells) in combination with one or more pharmaceutically or physiologically acceptable carriers, diluents, or excipients.

The present disclosure, among other things, also provides pharmaceutical compositions comprising at least one KIR3DL3 inhibitor in combination with one or more pharmaceutically or physiologically acceptable carriers, diluents, or excipients. In some embodiments, a pharmaceutical composition comprises an anti-KIR3DL3 antibody or antigen-binding fragment thereof in combination with one or more pharmaceutically or physiologically acceptable carriers, diluents, or excipients. In some embodiments, a pharmaceutical composition comprises a population of modified immune effector cells described herein and at least one KIR3DL3 inhibitor described herein.

When “a therapeutically effective amount, “an immunologically effective amount,” “an anti-immune response effective amount,” or “an immune response-inhibiting effective amount” is indicated, a precise amount of a pharmaceutical composition comprising a population of modified immune effector cells described herein (e.g., NK cells or T cells) and/or a pharmaceutical composition comprising at least one KIR3DL3 inhibitor as described herein (e.g., an anti-KIR3DL3 antibody or antigen-binding fragment thereof) can be determined by a physician with consideration of individual differences in age, weight, immune response, and condition of the patient (subject).

Pharmaceutical compositions described herein may comprise buffers including neutral buffered saline or phosphate buffered saline (PBS); carbohydrates, such as glucose, mannose, sucrose, dextrans, or mannitol; proteins, polypeptides, or amino acids (e.g., glycine); antioxidants; chelating agents, such as EDTA or glutathione; adjuvants (e.g., aluminum hydroxide); and preservatives. In some embodiments, a pharmaceutical composition is substantially free of contaminants, e.g., there are no detectable levels of a contaminant (e.g., an endotoxin).

Pharmaceutical compositions described herein may be administered in a manner appropriate to the disease, disorder, or condition to be treated or prevented. Quantity and frequency of administration will be determined by such factors as condition of a patient, and type and severity of a patient's disease, disorder, or condition, although appropriate dosages may be determined by clinical trials.

Pharmaceutical compositions described herein may be in a variety of forms. These include, for example, liquid, semi-solid and solid dosage forms, such as liquid solutions (e.g., injectable and infusible solutions), dispersions or suspensions, liposomes, and suppositories. Preferred compositions may be injectable or infusible solutions. Pharmaceutical compositions described herein can be formulated for administration intravenously, subcutaneously, intradermally, intratumorally, intranodally, intramedullary, intramuscularly, transarterially, or intraperitoneally.

In some embodiments, a pharmaceutical composition described herein is formulated for parenteral (e.g., intravenous, subcutaneous, intraperitoneal, or intramuscular) administration. In some embodiments, a pharmaceutical composition described herein is formulated for subcutaneous, intravenous, intramuscular, or intrasternal injection or infusion. In preferred embodiments, a pharmaceutical composition described herein is formulated for subcutaneous or intravenous injection of infusion. Pharmaceutical compositions described herein can be formulated for administered by using infusion techniques that are commonly known in immunotherapy (See, e.g., Rosenberg et al., New Eng. J. of Med. 319:1676, 1988, which is hereby incorporated by reference in its entirety).

As used herein, the terms “parenteral administration” and “administered parenterally” refer to modes of administration other than enteral and topical administration, usually by injection or infusion, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intratumoral, and intrasternal injection and infusion.

Pharmaceutical compositions comprising modified immune effector cells described herein (e.g., NK cells or T cells) may be administered at a dosage of about 10⁴ to about 10⁹ cells/kg body weight (e.g., about 10⁵ to about 10⁶ cells/kg body weight), including all integer values within those ranges). In some embodiments, a dose of modified immune effector cells described herein (e.g., NK cells or T cells) comprises at least about 1×10⁶, about 1.1×10⁶, about 2×10⁶, about 3.6×10⁶, about 5×10⁶, about 1×10⁷, about 1.8×10⁷, about 2×10⁷, about 5×10⁷, about 1×10⁸, about 2×10⁸, about 5×10⁸, about 1×10⁹, about 2×10⁹, or about 5×10⁹ cells. Pharmaceutical compositions described herein may also be administered multiple times at a certain dosage. An optimal dosage and treatment regime for a particular patient can readily be determined by one skilled in the art by monitoring a patient for signs of a disease, disorder, or condition and adjusting treatment accordingly.

In some embodiments, pharmaceutical compositions described herein are administered in combination with (e.g., before, simultaneously, or following) bone marrow transplantation or lymphocyte ablative therapy using a chemotherapy agent (e.g., fludarabine, external-beam radiation therapy (XRT), cyclophosphamide, or Rituxan). In certain embodiments, subjects undergo standard treatment with high dose chemotherapy followed by peripheral blood stem cell transplantation. In certain embodiments, following transplant, subjects receive an infusion of one or more pharmaceutical compositions described herein. In some embodiments, pharmaceutical compositions described herein may be administered before or following surgery.

A dosage of any aforementioned therapy to be administered to a subject will vary with a disease, disorder, or condition being treated and based on a specific subject. Scaling of dosages for human administration can be performed according to art-accepted practices.

### Kits

The present disclosure, among other things, provides kits comprising at least one KIR3DL3 inhibitor described herein, and instructions for use and/or administration. In some embodiments, kits may include one or more containers comprising a pharmaceutical composition that comprises at least one anti-KIR3DL3 antibody or antigen-binding fragment thereof described herein, and instructions for use and/or administration.

The present disclosure, among other things, provides kits comprising at least one immune cell activating agent described herein, at least one KIR3DL3 inhibitor described herein, and instructions for use and/or administration. Such kits may include one or more containers comprising a first pharmaceutical composition that comprises at least one immune cell activating agent described herein, and a pharmaceutically acceptable carrier, and a second pharmaceutical composition that comprises at least one KIR3DL3 inhibitor described herein and a pharmaceutically acceptable carrier. In some embodiments, the kits described herein comprise one or more anti-KIR3DL3 antibody or antigen-binding fragment thereof, described herein, and instructions for use and/or administration.

The present disclosure, among other things, provides kits comprising a population of modified immune effector cells and at least one KIR3DL3 inhibitor, and instructions for use and/or administration. Such kits may include one or more containers comprising a first pharmaceutical composition that comprises a population of modified immune effector cells described herein, and a pharmaceutically acceptable carrier, and a second pharmaceutical composition that comprises at least one KIR3DL3 inhibitor described herein and a pharmaceutically acceptable carrier. In some embodiments, such kits comprise at least one KIR3DL3 antibody or antigen-binding fragment thereof described herein and a pharmaceutically acceptable carrier.

In some embodiments, a kit comprises instructions for use in any method described herein. Instructions can comprise a description of administration of the first and second pharmaceutical compositions to a subject to achieve the intended activity in a subject. The kit may further comprise a description of selecting a subject suitable for treatment based on identifying whether the subject is in need of the treatment. In some embodiments, the instructions comprise a description of administering the first and second pharmaceutical compositions to a subject who is in need of the treatment.

The instructions relating to the first and second pharmaceutical compositions described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease, disorder or condition in a subject.

The kits provided herein are in suitable packaging. Suitable packaging includes, but is not limited to, vials, bottles, jars, flexible packaging, and the like. Also contemplated are packages for use in combination with a specific device, such as an infusion device. A kit may have a sterile access port (for example, the container may be an intravenous solution bag or a vial having a stopper pierce able by a hypodermic injection needle). The container may also have a sterile access port.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### INCORPORATION BY REFERENCE

All publications, patent applications, patents, and other references mentioned herein, including GenBank Accession Numbers, are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLE

The disclosure is further illustrated by the following example. An example is provided for illustrative purposes only. It is not to be construed as limiting the scope or content of the disclosure in any way.

### Example 1: First-In-Class Monoclonal Antibody that Binds Human KIR3DL3

To explore the therapeutic potential of HHLA2-KIR3DL3 blockade, we generated a first-in-class, monoclonal antibody called NPX267 (Hu26E10z7p7 in Table 1) that binds with high affinity to human killer cell immunoglobulin like receptor, three Ig domains and long cytoplasmic tail 3 (KIR3DL3). KIR3DL3 is a member of the killer cell Ig-like (KIR) receptor family and is expressed by both NK and T cells. KIR3DL3 has recently been shown to be a coinhibitory receptor for the B7 ligand, human endogenous retrovirus H long terminal repeat-associating protein 2 (HHLA2) (see Bhatt et al, Cancer Immunol Res 2021; 9: 156-169 and Wei et al. Science Immunol. 2021; 6: eab9792; each of which is hereby incorporated by reference in its entirety).

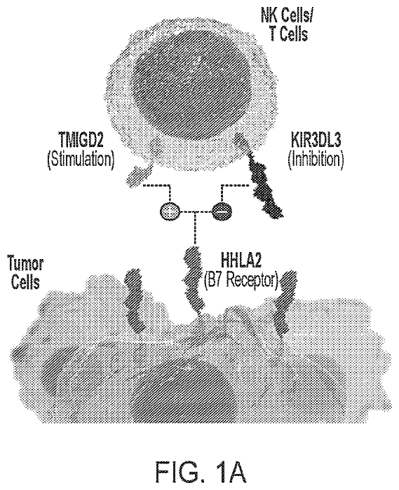

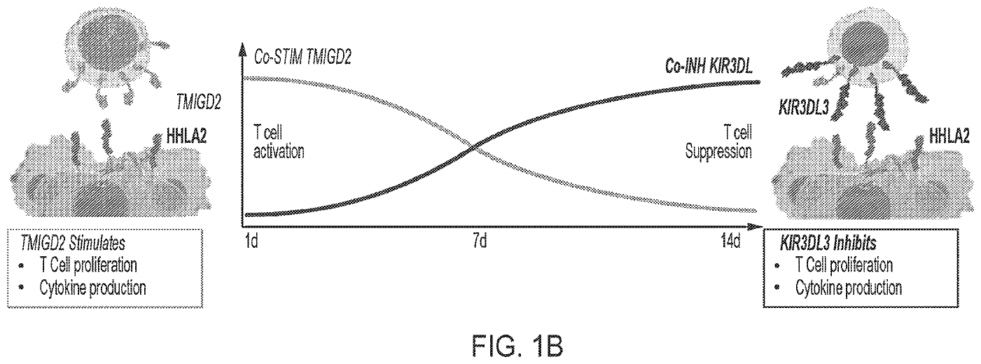

KIR3DL3, expressed on T and NK cells in the tumor microenvironment, suppresses immune responses following engagement with HHLA2 (FIGS. 1A and 1B). Upon HHLA2-induced KIR3DL3 activation, SHP-1 and SHP-2 are recruited to KIR3DL3's cytoplasmic immunoreceptor tyrosine-based inhibitory motif (ITIM) and downstream activation signals are blunted. As a result, T and NK cell activity is suppressed. HHLA2 has limited expression in normal tissues, but is highly expressed in many cancers and is often associated with poor patient outcomes. In renal cell carcinoma (RCC), HHLA2 expression is often not co-expressed with PD-L1. However, co-expression of HHLA2 and PD-L1 in tumors from patients with RCC is associated with worse progression free survival that those with tumors exclusively expressing PD-L1. Thus, the KIR3DL3-HHLA2 axis represents a novel immune checkpoint pathway and blockade of KIR3DL3 signaling may be a promising strategy to promote antitumor immunity.

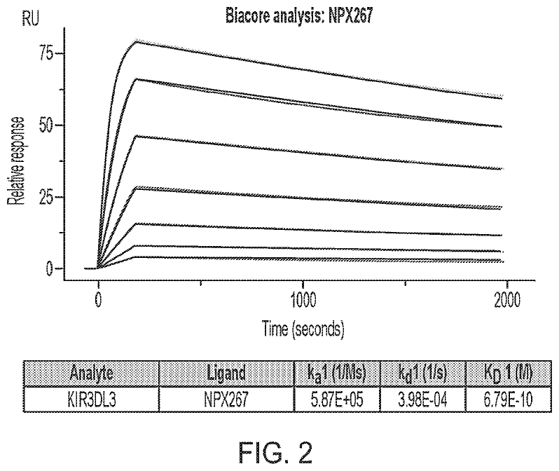

NPX267 is a humanized IgG4 monoclonal antibody that was shown to bind specifically to KIR3DL3 (FIG. 2). The monovalent binding affinity of NPX267 to recombinant KIR3DL3 protein was determined by SPR using a Biacore instrument; KIR3DL3 protein was immobilized on a CM5 chip and NPX267 was run as the analyte. The affinity of NPX267 for human KIR3DL3 was 679 pM. No off-target binding was found in a screen of greater than 5800 plasma membrane proteins.

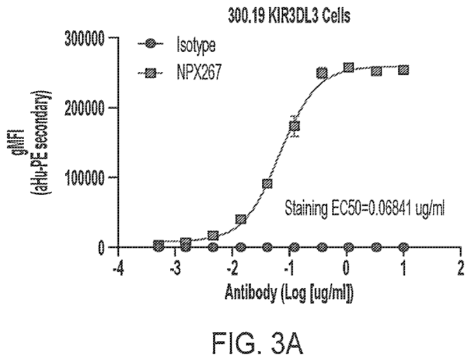

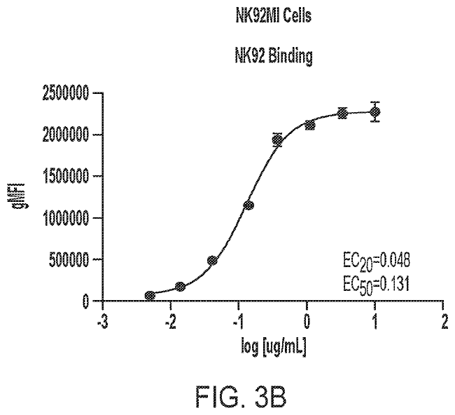

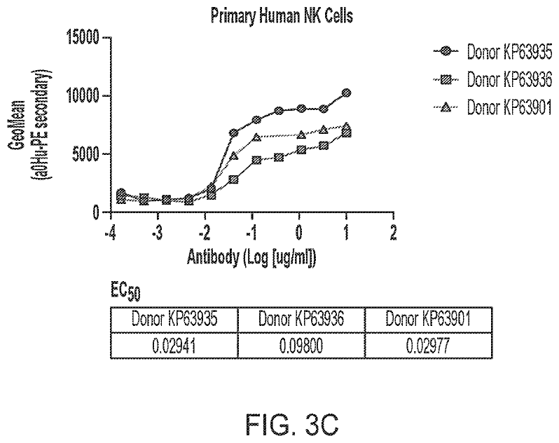

NPX267 was shown to specifically bind to KIR3DL3 expressed on 300.19-KIR3DL3 cells, NK92MI cells, and primary human NK cells (FIGS. 3A-3C). 300.19-KIR3DL3 cells were treated with NPX267 or IgG4 isotype control antibody at concentrations ranging from 10 mg/mL to 0.0005 mg/mL for 30 minutes (FIG. 3A). Flow cytometric analysis of primary antibody binding was detected using an anti-human PE secondary antibody. The mean and standard deviation of duplicate data points are shown. Data performed in duplicate was entered into GraphPad Prism software and the EC₅₀ was determined using a 4-parameter nonlinear regression equation.

NK92 MI cells were treated with NPX267 at concentrations ranging from 10 mg/mL to 0.0005 mg/mL for 30 minutes (FIG. 3B). Flow cytometric analysis of primary antibody binding was detected using an anti-human PE secondary antibody. The mean and standard deviation of triplicate data points are shown.

For primary human NK cells, human whole blood cells were treated with NPX267 at concentrations ranging from 10 mg/mL to 0.00016351 mg/mL for 30 minutes (FIG. 3C). Red blood cells were lysed and cells were stained with a cocktail of anti-human CD3, Anti-human CD56 and anti-human IgG4 PE. Flow cytometric analysis of primary antibody binding was detected using an anti-human PE secondary antibody.

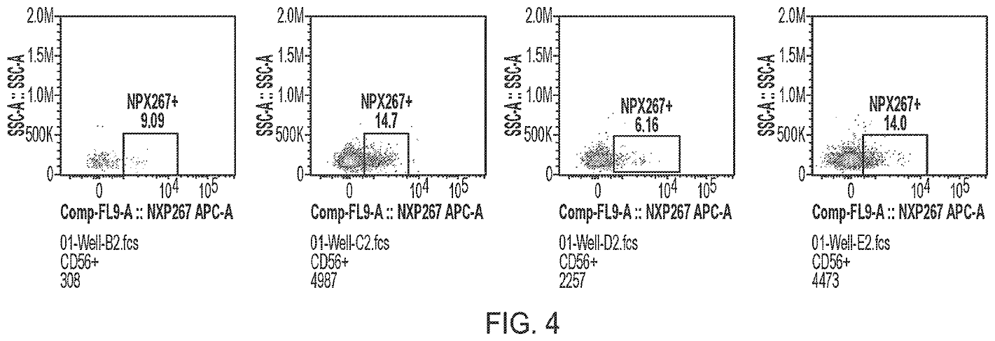

NPX267 was shown to bind to KIR3DL3 on tumor infiltrating CD56+ NK cells (FIG. 4). Cryopreserved human dissociated tumor cells were thawed and split into NPX276 and fluorescence-minus-one (FMO) conditions. Cells were stained with an antibody cocktail of Viability dye, CD45-BV421, EpCAM-FITC and CD56-PE for 30 minutes+/−NPX267-APC. Samples were acquired immediately on a flow cytometer. NPX267+ gates were determined from FMO sample for each donor. KIR3DL3 expression ranged from 6 to 15% of infiltrating CD56+ NK cells.

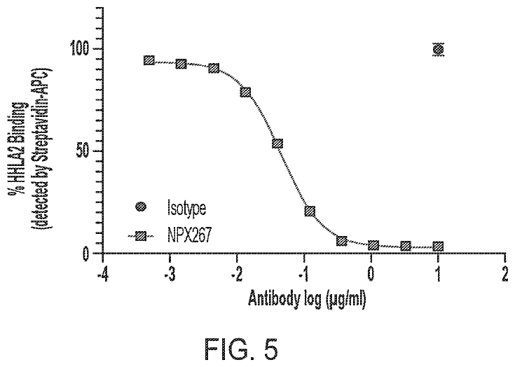

NPX267 was shown to block binding of KIR3DL3 to HHLA2 (FIG. 5). 300.19-KIR3DL3 cells were treated with NPX267 or IgG4 isotype control antibody at concentrations ranging from 10 mg/mL to 0.0005 mg/mL for 30 minutes. Recombinant biotinylated HHLA2 at 5 mg/mL was added to the cells and incubated for 30 minutes. The cells were treated with 1:250 of APC-streptavidin for 30 minutes to detect recombinant biotinylated HHLA2 bound to cells. The gMFI of APC-streptavidin on 300.19-KIR3DL3 cells was measured by flow cytometry and percent inhibition was calculated. Samples treated with FACS buffer only, recombinant biotinylated HHLA2 protein, and APC-streptavidin were used to establish the maximum binding signal (0% inhibition), while samples treated with biotinylated HHLA2 without addition of APC-streptavidin were used to set the minimum binding signal (100% inhibition). NPX267 blocked recombinant HHLA2-Fc from binding to its inhibitory receptor KIR3DL3 in a dose dependent manner.

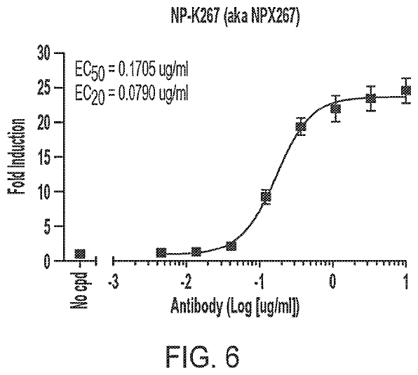

NPX267 was shown to block HHLA2-mediated suppressive activity in a T cell reporter assay (FIG. 6). HHLA2/TCR/CHO cells were seeded into white, clear-bottom 96-well plates and allowed to adhere overnight. The next day Jurkat/IL-2/KIR3DL3 cells were incubated with NPX267 for 1 hour. Tissue culture medium was removed from the HHLA2/TCR/CHO cells, and Jurkat/IL-2/KIR3DL3 cells pre-complexed with experimental antibodies and an anti-CD28 agonist antibody were added for 5-6 hours. The One-Step Luciferase Assay System was used to perform the luciferase assay. Luminescence was measured using a luminometer (BioTek Synergy™ 2 microplate reader). All treatment conditions were performed in triplicate. The luminescence intensity data were analyzed using GraphPad Prism software. In the absence of the compound, the luminescence intensity (Lt) in each data set was defined as 1. The fold induction of luminescence in the presence of each compound was calculated according to the following equation: fold induction=(L−Lb)/(Lt−Lb), where L=the luminescence intensity in the presence of the compound, Lb=the luminescence intensity in the absence of cells, and Lt=the luminescence intensity in the absence of the compound. Fold induction values were plotted in GraphPad Prism software and IC₅₀ was determined using a 4-parameter nonlinear regression equation.

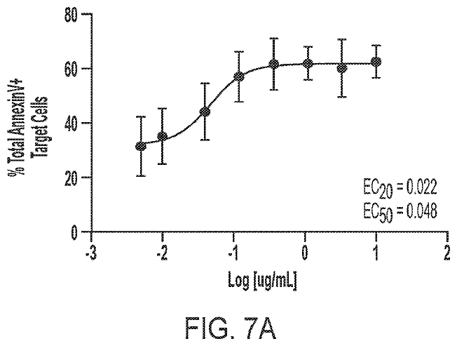

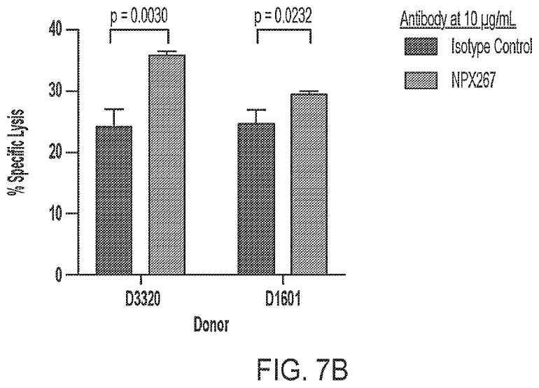

NPX267 was shown to enhance NK cell killing of HHLA2 expressing tumor cells (FIGS. 7A and 7B). NK92MI effector cells were plated in a round bottom 96-well plate and treated with 10 mg/mL NPX267 for 30 minutes. Following treatment, NK92MI cells were mixed with K562 cells engineered to express HHLA2 at a 1:1 effector to target ratio and incubated at 37C and 5% CO2 for 3 hours. Cells were then stained with the apoptosis marker Annexin V and analyzed via flow cytometry to assess target cell death. NPX267 increased target cell death by a maximum of two-fold in a dose dependent manner over an IgG4 isotype control (FIG. 7A).

KIR3DL3+ human NK cells were incubated with 10 mg/mL of NPX267 or IgG4 isotype control for 30 minutes then added to CellTrace Violet labelled HCC827 cells at a ratio of 5 NK cells to 1 HCC827 cell for 6 hours. Cells were stained with 7-AAD. Flow cytometry was used to detect non-viable HCC827 cells (CellTrace Violet+7-AAD+). Specific lysis was calculated by Specific lysis=(CellTrace Violet+7-AAD+ cells)/(CellTrace Violet+ cells)*100. The mean and standard deviation of triplicate data points for each condition are shown. P-values were calculated using a two-sided Student's T-Test. NPX267 increased target cell death in two different donors with varying levels of KIR3DL3 (FIG. 7B).

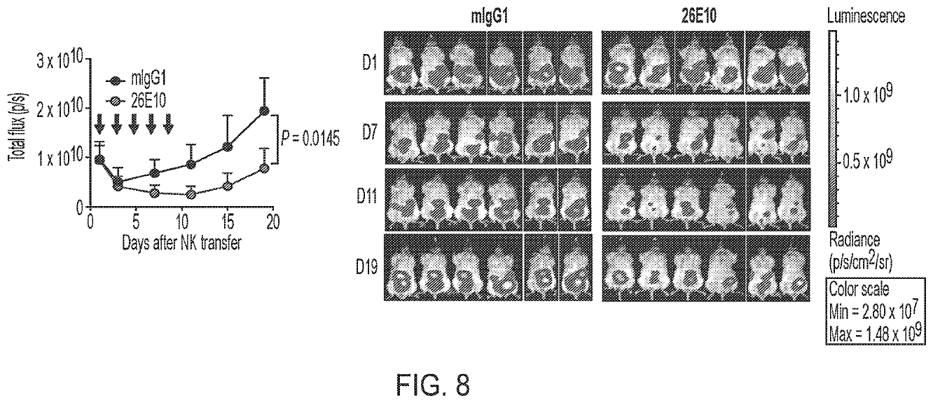

NPX267 is the humanized version of 26E10, which was shown to enhance NK cell-mediated anti-tumor activity in the HCC827 model in vivo (FIG. 8). Luciferase tagged HCC827 cells (4×10⁶) were injected intraperitoneally (ip) into 6-8 week old NSG mice (n=6/group). When tumors were established, mice were injected ip with KIR3DL3+ NK cells (1×10⁷) together with 1 μg of rhIL-2, 1 μg of rhIL-15, and 200 μg of NPX267 (or mIgG1) every other day for a total of 5 injections. Tumor growth was assessed by imaging.

In summary, the data presented herein demonstrate that NPX267 blocked HHLA2 engagement with KIR3DL3 on primary human NK and T cells in a dose dependent fashion. The ability of NPX267 to block KIR3DL3-mediated suppression of T cell activation was assessed with a T cell reporter system and primary CD8+ T cell functional assays. KIR3DL3 blockade with NPX267 inhibited HHLA2-mediated suppression of T cell activation in a dose dependent manner. The anti-tumor activity of KIR3DL3 blockade with NPX267 was also demonstrated in NK-cell mediated cytotoxicity assays. NPX267 treatment augmented the ability of human NK cell lines and primary NK cells to kill HHLA2⁺ tumor cells in vitro. Finally, NPX267 blockade of HHLA2 mediated KIR3DL3 signaling enhanced anti-tumor immunity in humanized mouse models bearing HHLA2⁺ human tumors.

Collectively, these data demonstrate that the KIR3DL3-HHLA2 pathway is a novel immune checkpoint axis that facilitates tumor escape by attenuating both innate and adaptive antitumor immune responses. NPX267, a first-in-class KIR3DL3 blocking antibody, potentiates anti-tumor immunity against HHLA2⁺ tumors and represents a promising approach to treat certain diseases, disorders or conditions, and particularly cancers.

### EQUIVALENTS

It is to be appreciated by those skilled in the art that various alterations, modifications, and improvements to the present disclosure will readily occur to those skilled in the art. Such alterations, modifications, and improvements are intended to be part of the present disclosure, and are intended to be within the spirit and scope of the invention. Accordingly, the foregoing description and drawing are by way of example only and any invention described in the present disclosure if further described in detail by the claims that follow.

Those skilled in the art will appreciate typical standards of deviation or error attributable to values obtained in assays or other processes described herein. The publications, websites and other reference materials referenced herein to describe the background of the invention and to provide additional detail regarding its practice are hereby incorporated by reference in their entireties.

1. A method of treating a subject having a disease, disorder or condition comprising administering a population of modified immune effector cells,
wherein, prior to administering, a population of immune effector cells were contacted with at least one immune cell activating agent and at least one KIR3DL3 inhibitor, thereby forming a population of modified immune effector cells.

2. A method of treating a subject having a disease, disorder, or condition comprising:
(i) administering a population of modified immune effector cells to the subject, wherein a population of immune effector cells were contacted with at least one immune cell activating agent prior to administering, thereby forming a population of modified immune effector cells, and
(ii) administering at least one KIR3DL3 inhibitor to the subject.

3. The method of claim 1 or 2, wherein the at least one KIR3DL3 inhibitor is or comprises an anti-KIR3DL3 antibody or an antigen-binding fragment thereof, a miRNA, a shRNA, a siRNA, a CRISPR/Cas guide system, a TALEN, a ZFN, and/or a demethylating agent.

4. The method of claim 3, wherein the antigen-binding fragment comprises an scFv, Fab, Fab′, F(ab′)2, Fc, or nanobody.

5. The method of claim 3 or 4, wherein the anti-KIR3DL3 antibody or an antigen-binding fragment thereof comprises:
(a) a heavy chain variable region (VH) comprising one, two, or three VH CDR sequences each with at least about 90% identity to a VH CDR of Table 1; and/or
(b) a light chain variable region (VL) comprising one, two, or three VL CDR sequences each with at least about 90% identity to a VL CDR of Table 1.

6. The method of claim 3, wherein the demethylating agent comprises or is 5-aza-2-deoxycytidine (Aza), 5-azacytidine, 1-β-D-arabinofuranosil-5-azacytosine, or dihydro-5-azacytidine.

7. The method of any one of claims 1-6, wherein the immune cell activating agent results in T cell proliferation and/or increased endogenous expression of at least one cytokine.

8. The method of any one of claims 1-7, wherein the immune cell activating agent comprises or is a cytokine agent.

9. The method of claim 8, wherein the cytokine agent is or comprises IL-2, IL-15, IL-12, IL-17, IL-18, and/or IL-21, IFNγ, and/or TNFα.

10. The method of claim 9, wherein the IL-2 binds to IL-2Rα, IL-2Rβ, or IL-2Rγ.

11. The method of claim 9 or 10, wherein the IL-2 expands only T cells and does not substantially expand Tregs.

12. The method of claim 8, wherein the cytokine agent is or comprises an inhibitor of a suppressor of cytokine signaling (SOCS) protein.

13. The method of any one of claims 1-7, wherein the immune cell activating agent comprises or is a costimulatory antibody or antigen binding fragment thereof, small molecule, polypeptide, glycoprotein, or exogenous cell.

14. The method of claim 13, wherein the costimulatory antibody or antigen binding fragment thereof or costimulatory small molecule binds to 4-1BB, CD3, CD40, CD28, OX40, GITR, CTLA-4, PD-1, PD-L1, PD-L2, TIM-3, TGF-β or LAG-3, CD39, or CD73.

15. The method of claim 14, wherein the costimulatory antibody or antigen binding fragment thereof comprises or is OKT3.

16. The method of claim 13, wherein the costimulatory polypeptide comprises or is a soluble HHLA2 Fc fusion polypeptide.

17. The method of claim 13, wherein the costimulatory glycoprotein comprises or is a fibronectin protein or fragment thereof.

18. The method of claim 13, wherein the exogenous cell comprises or is an artificial antigen presenting cell.

19. The method of any one of claims 1-18, wherein the immune effector cells are isolated from peripheral blood mononuclear cells (PBMCs) or tumor cells.

20. The method of any one of claims 1-19, wherein the modified immune effector cells comprise or are NK cells and/or T cells.

21. The method of claim 20, wherein the T cells comprise or are CD4+ T cells and/or CD8+ T cells.

22. The method of any one of claims 1-21, wherein the modified immune effector cells comprise at least one CAR.

23. The method of any one of claims 1-22, wherein the modified immune effector cells are administered to the subject within less than about 3 hours of contacting with the at least one KIR3DL3 inhibitor

24. The method of claim 23, wherein the modified immune effector cells are administered to the subject within less than about 1 minute, about 2 minutes, about 3 minutes, about 4 minutes, about 5 minutes, about 10 minutes, about 30 minutes, or about 45 minutes, about 1 hour, about 1.5 hours, about 2 hours, about 2.5 hours, or about 3 hours of contacting with the at least one KIR3DL3 inhibitor.

25. The method of any one of claims 1-24, wherein the population of modified immune effector cells and/or the KIR3DL3 inhibitor is administered parenterally.

26. The method of claim 25, wherein parenteral administration is or comprises subcutaneous, intravenous, intramuscular, or intrasternal injection or infusion.

27. The method of any one of claims 1-26, wherein the method comprises sequential administration of the population of modified immune effector cells and the at least one KIR3DL3 inhibitor.

28. The method of claim 27, wherein:
(i) the population of modified immune effector cells are administered prior to administration of the at least one KIR3DL3 inhibitor; or
(ii) the population of modified immune effector cells are administered after administration of the at least one KIR3DL3 inhibitor.

29. The method of any one of claims 1-26, comprising co-administration of the population of modified immune effector cells and the at least one KIR3DL3 inhibitor.

30. The method of claim 29, wherein the method comprises co-administration by injection.

31. The method of any one of claims 1-30, wherein the subject has a cancer.

32. The method of claim 31, wherein the subject has a solid tumor.

33. The method of claim 32, wherein the solid tumor is or comprises one or more of: a renal cancer, a bone cancer, a skin cancer, a breast cancer, a cervical cancer, a colorectal cancer, an endometrial cancer, a lung cancer, an ovarian cancer, a liver cancer, cholangiocarcinoma, or a thyroid cancer.

34. The method of claim 31, wherein the subject has a hematological cancer.

35. The method of claim 34, wherein the hematological cancer comprises or is a leukemia or lymphoma.

36. The method of claim 35, wherein the leukemia comprises or is acute lymphocytic leukemia, acute myeloid leukemia, chronic myeloid leukemia, acute lymphoblastic leukemia, chronic lymphocytic leukemia, chronic leukemia, or acute leukemia.

37. The method of claim 35, wherein the lymphoma comprises or is Hodgkin lymphoma (HL), non-Hodgkin's lymphoma, lymphocytic lymphoma, or diffuse large B cell lymphoma (DLBCL).

38. The method of any one of claims 31-37, wherein the subject has a cancer that is resistant to a treatment comprising a cytokine agent.

39. The method of any one of claims 1-38, further comprising determining expression of TMIGD2 and/or KIR3DL3 by the modified immune effector cells.

40. The method of any one of claims 1-39, further comprising determining activation of the immune effector cells.

41. The method of any one of claims 1-40, further comprising determining expression of CD25, CD69, CD137, CD16, CD56, CD96, CD226, KIR2DL5, and/or NKG2D.

42. The method of any one of claims 1-41, further comprising formulating the population of modified immune cells into a composition for administration to a subject.

43. A method of making a population of modified immune effector cells comprising:
(i) contacting a population of immune effector cells with at least one immune cell activating agent, and
(ii) contacting the population of immune effector cells with at least one KIR3DL3 inhibitor, thereby creating a population of modified immune effector cells.

44. A composition comprising a population of modified immune effector cells, at least one immune cell activating agent, and at least one KIR3DL3 inhibitor.

45. A composition comprising a population of modified immune effector cells and at least one KIR3DL3 inhibitor, wherein the immune effector cells were contacted with at least one immune cell activating agent.

46. A kit comprising at least one immune cell activating agent, at least one KIR3DL3 inhibitor, and instructions for use and/or administration.

47. A kit comprising a population of modified immune effector cells and at least one KIR3DL3 inhibitor, and instructions for use and/or administration, wherein the immune effector cells were contacted with at least one immune cell activating agent.

48. An anti-KIR3DL3 antibody or an antigen-binding fragment thereof, which is or comprises:
(a) a heavy chain variable region (VH) comprising one, two, or three VH CDR sequences each with at least about 90% identity to a VH CDR of Table 1; and/or
(b) a light chain variable region (VL) comprising one, two, or three VL CDR sequences each with at least about 90% identity to a VL CDR of Table 1.

49. The anti-KIR3DL3 antibody or antigen-binding fragment thereof of claim 48, which is or comprises:
(a) a VH comprising one, two, or three VH CDR sequences each with at least about 91%, 92%, 93%, 94%, 95%, 96%, 97%, 98%, 99%, 99.5% or higher identity to a VH CDR of Table 1; and/or
(b) a VL comprising one, two, or three VL CDR sequences each with at least about 91%, 92%, 93%, 94%, 95%, 96%, 97%, 98%, 99%, 99.5% or higher identity to a VL CDR of Table 1.

50. The anti-KIR3DL3 antibody or antigen-binding fragment thereof of claim 48 or 49, which is or comprises:
(a) a VH with at least about 90% or more identity to a VH of Table 1; and/or
(b) a VL with at least about 90% or more identity to a VL of Table 1.

51. The anti-KIR3DL3 antibody or antigen-binding fragment thereof of claim 50, which is or comprises:
(a) a VH with at least about 95%, 96%, 97%, 98%, 99%, 99.5% or higher identity to a VH of Table 1; and/or
(b) a VL with at least about 95%, 96%, 97%, 98%, 99%, 99.5% or higher identity to a VL of Table 1.

52. The anti-KIR3DL3 antibody or antigen-binding fragment thereof of any one of claims 48-51, which is or comprises:
(a) a heavy chain with at least about 90% or more identity to a heavy chain of Table 1; and/or
(b) a light chain with at least about 90% or more identity to a light chain of Table 1.

53. The anti-KIR3DL3 antibody or antigen-binding fragment thereof of claim 52, which is or comprises:
(a) a heavy chain with at least about 95%, 96%, 97%, 98%, 99%, 99.5% or higher identity to a heavy chain of Table 1; and/or
(b) a light chain with at least about 95%, 96%, 97%, 98%, 99%, 99.5% or higher identity to a light chain of Table 1.

54. A nucleic acid encoding the anti-KIR3DL3 antibody or antigen-binding fragment thereof of any one of claims 48-53.

55. An expression vector comprising the nucleic acid of claim 54.

56. A host cell comprising or expressing the anti-KIR3DL3 antibody or antigen-binding fragment thereof of any one of claims 48-53, comprising the nucleic acid of claim 54, or comprising the expression vector of claim 55.

57. A pharmaceutical composition comprising at least one anti-KIR3DL3 antibody or antigen-binding fragment thereof of any of the above claims, and a pharmaceutically acceptable carrier, diluent, or excipient.

58. A method of treating a subject having a disease, disorder, or condition comprising: administering a therapeutically effective amount of a pharmaceutical composition of claim 57.

59. A method of modulating an immune response in a subject comprising: administering a therapeutically effective amount of a pharmaceutical composition of claim 57.

60. The method of claim 58 or 59, wherein the subject has or is at risk of developing a cancer.

61. The method of claim 60, wherein the subject has a solid tumor or a hematological cancer.

62. The method of claim 61, wherein the solid tumor is or comprises one or more of: a renal cancer, a bone cancer, a skin cancer, a breast cancer, a cervical cancer, a colorectal cancer, an endometrial cancer, a lung cancer, an ovarian cancer, a liver cancer, cholangiocarcinoma, or a thyroid cancer.

63. The method of claim 61, wherein the hematological cancer comprises or is a leukemia or lymphoma.

64. The method of claim 63, wherein the leukemia comprises or is acute lymphocytic leukemia, acute myeloid leukemia, chronic myeloid leukemia, acute lymphoblastic leukemia, chronic lymphocytic leukemia, chronic leukemia, or acute leukemia.

65. The method of claim 63, wherein the lymphoma comprises or is Hodgkin lymphoma (HL), non-Hodgkin's lymphoma, lymphocytic lymphoma, or diffuse large B cell lymphoma (DLBCL).


SEQUENCE LISTING (78 sequences)

SEQ ID NO: 1 - protein, 8 aa, synthetic construct
GYTFTSYN

SEQ ID NO: 2 - protein, 8 aa, synthetic construct
IYPGDAVT

SEQ ID NO: 3 - protein, 14 aa, synthetic construct
ARSGNYGNYEGFAY

SEQ ID NO: 4 - protein, 5 aa, synthetic construct
SYNIH

SEQ ID NO: 5 - protein, 17 aa, synthetic construct
YIYPGDAVTNYNQKFKG

SEQ ID NO: 6 - protein, 12 aa, synthetic construct
SGNYGNYEGFAY

SEQ ID NO: 7 - protein, 10 aa, synthetic construct
GYTFTSYNIH

SEQ ID NO: 8 - protein, 17 aa, synthetic construct
YIYPGDAVTNYNQKFKG

SEQ ID NO: 9 - protein, 12 aa, synthetic construct
SGNYGNYEGFAY

SEQ ID NO: 10 - protein, 121 aa, synthetic construct
QAQLVQSGAEVKKPGASVKVSCKASGYTFTSYNIHWVRQAPGQGLEWMGYIYPGDAVTNYNQKFKGRVTMTADTSTSTVYMELSSLRSEDTAVYYCARSGNYGNYEGFAYWGQGTLVTVSS

SEQ ID NO: 11 - DNA, 363 bp, synthetic construct
  source 1..363: mol_type = other DNA
caggcgcagctggtgcagagcggcgcggaagtgaaaaaaccgggcgcgagcgtgaaagtgagctgcaaagcgagcggctatacctttaccagctataacattcattgggtgcgccaggcgccgggccagggcctggaatggatgggctatatttatccgggcgatgcggtgaccaactataaccagaaatttaaaggccgcgtgaccatgaccgcggataccagcaccagcaccgtgtatatggaactgagcagcctgcgcagcgaagataccgcggtgtattattgcgcgcgcagcggcaactatggcaactatgaaggctttgcgtattggggccagggcaccctggtgaccgtgagcagc

SEQ ID NO: 12 - protein, 448 aa, synthetic construct
QAQLVQSGAEVKKPGASVKVSCKASGYTFTSYNIHWVRQAPGQGLEWMGYIYPGDAVTNYNQKFKGRVTMTADTSTSTVYMELSSLRSEDTAVYYCARSGNYGNYEGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - DNA, 1344 bp, synthetic construct
  source 1..1344: mol_type = other DNA
caggcgcagctggtgcagagcggcgcggaagtgaaaaaaccgggcgcgagcgtgaaagtgagctgcaaagcgagcggctatacctttaccagctataacattcattgggtgcgccaggcgccgggccagggcctggaatggatgggctatatttatccgggcgatgcggtgaccaactataaccagaaatttaaaggccgcgtgaccatgaccgcggataccagcaccagcaccgtgtatatggaactgagcagcctgcgcagcgaagataccgcggtgtattattgcgcgcgcagcggcaactatggcaactatgaaggctttgcgtattggggccagggcaccctggtgaccgtgagcagcgcgagcaccaaaggcccgagcgtgtttccgctggcgccgtgcagccgcagcaccagcgaaagcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcaccaaaacctatacctgcaacgtggatcataaaccgagcaacaccaaagtggataaacgcgtggaaagcaaatatggcccgccgtgcccgccgtgcccggcgccggaatttctgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttaacagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaa

SEQ ID NO: 14 - protein, 6 aa, synthetic construct
QDTQSY

SEQ ID NO: 15 - skipped

SEQ ID NO: 16 - protein, 9 aa, synthetic construct
LQYDEFPYT

SEQ ID NO: 17 - protein, 11 aa, synthetic construct
KASQDTQSYLS

SEQ ID NO: 18 - protein, 7 aa, synthetic construct
RANRLVD

SEQ ID NO: 19 - protein, 9 aa, synthetic construct
LQYDEFPYT

SEQ ID NO: 20 - protein, 11 aa, synthetic construct
KASQDTQSYLS

SEQ ID NO: 21 - protein, 7 aa, synthetic construct
RANRLVD

SEQ ID NO: 22 - protein, 9 aa, synthetic construct
LQYDEFPYT

SEQ ID NO: 23 - protein, 107 aa, synthetic construct
DIQMTQSPSSLSASVGDRVTITCKASQDTQSYLSWFQQKPGKAPKTLIYRANRLVDGVPSRFSGSGSGTDFTLTISSLQPEDFATYYCLQYDEFPYTFGGGTKVEIK

SEQ ID NO: 24 - DNA, 321 bp, synthetic construct
  source 1..321: mol_type = other DNA
gatattcagatgacccagagcccgagcagcctgagcgcgagcgtgggcgatcgcgtgaccattacctgcaaagcgagccaggatacccagagctatctgagctggtttcagcagaaaccgggcaaagcgccgaaaaccctgatttatcgcgcgaaccgcctggtggatggcgtgccgagccgctttagcggcagcggcagcggcaccgattttaccctgaccattagcagcctgcagccggaagattttgcgacctattattgcctgcagtatgatgaatttccgtatacctttggcggcggcaccaaagtggaaattaaa

SEQ ID NO: 25 - protein, 214 aa, synthetic construct
DIQMTQSPSSLSASVGDRVTITCKASQDTQSYLSWFQQKPGKAPKTLIYRANRLVDGVPSRFSGSGSGTDFTLTISSLQPEDFATYYCLQYDEFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 642 bp, synthetic construct
  source 1..642: mol_type = other DNA
gatattcagatgacccagagcccgagcagcctgagcgcgagcgtgggcgatcgcgtgaccattacctgcaaagcgagccaggatacccagagctatctgagctggtttcagcagaaaccgggcaaagcgccgaaaaccctgatttatcgcgcgaaccgcctggtggatggcgtgccgagccgctttagcggcagcggcagcggcaccgattttaccctgaccattagcagcctgcagccggaagattttgcgacctattattgcctgcagtatgatgaatttccgtatacctttggcggcggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgc

SEQ ID NO: 27 - protein, 8 aa, synthetic construct
GYTFTSYN

SEQ ID NO: 28 - protein, 8 aa, synthetic construct
IFPGDAGT

SEQ ID NO: 29 - protein, 14 aa, synthetic construct
ARSGYYGNYEGFAY

SEQ ID NO: 30 - protein, 5 aa, synthetic construct
SYNMH

SEQ ID NO: 31 - protein, 17 aa, synthetic construct
YIFPGDAGTNYNQKFKG

SEQ ID NO: 32 - protein, 12 aa, synthetic construct
SGYYGNYEGFAY

SEQ ID NO: 33 - protein, 10 aa, synthetic construct
GYTFTSYNMH

SEQ ID NO: 34 - protein, 17 aa, synthetic construct
YIFPGDAGTNYNQKFKG

SEQ ID NO: 35 - protein, 12 aa, synthetic construct
SGYYGNYEGFAY

SEQ ID NO: 36 - protein, 121 aa, synthetic construct
QVQLVQSGAEVKKPGASVKVSCKASGYTFTSYNMHWVRQAPGQGLEWMGYIFPGDAGTNYNQKFKGRVTMTADTSTSTVYMELSSLRSEDTAVYYCARSGYYGNYEGFAYWGQGTLVTVSS

SEQ ID NO: 37 - DNA, 363 bp, synthetic construct
  source 1..363: mol_type = other DNA
caggtgcagctggtgcagagcggcgcggaagtgaaaaaaccgggcgcgagcgtgaaagtgagctgcaaagcgagcggctatacctttaccagctataacatgcattgggtgcgccaggcgccgggccagggcctggaatggatgggctatatttttccgggcgatgcgggcaccaactataaccagaaatttaaaggccgcgtgaccatgaccgcggataccagcaccagcaccgtgtatatggaactgagcagcctgcgcagcgaagataccgcggtgtattattgcgcgcgcagcggctattatggcaactatgaaggctttgcgtattggggccagggcaccctggtgaccgtgagcagc

SEQ ID NO: 38 - protein, 448 aa, synthetic construct
QVQLVQSGAEVKKPGASVKVSCKASGYTFTSYNMHWVRQAPGQGLEWMGYIFPGDAGTNYNQKFKGRVTMTADTSTSTVYMELSSLRSEDTAVYYCARSGYYGNYEGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - DNA, 1344 bp, synthetic construct
  source 1..1344: mol_type = other DNA
caggtgcagctggtgcagagcggcgcggaagtgaaaaaaccgggcgcgagcgtgaaagtgagctgcaaagcgagcggctatacctttaccagctataacatgcattgggtgcgccaggcgccgggccagggcctggaatggatgggctatatttttccgggcgatgcgggcaccaactataaccagaaatttaaaggccgcgtgaccatgaccgcggataccagcaccagcaccgtgtatatggaactgagcagcctgcgcagcgaagataccgcggtgtattattgcgcgcgcagcggctattatggcaactatgaaggctttgcgtattggggccagggcaccctggtgaccgtgagcagcgcgagcaccaaaggcccgagcgtgtttccgctggcgccgtgcagccgcagcaccagcgaaagcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcaccaaaacctatacctgcaacgtggatcataaaccgagcaacaccaaagtggataaacgcgtggaaagcaaatatggcccgccgtgcccgccgtgcccggcgccggaatttctgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttaacagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaa

SEQ ID NO: 40 - protein, 6 aa, synthetic construct
QDIQSY

SEQ ID NO: 41 - skipped

SEQ ID NO: 42 - protein, 9 aa, synthetic construct
LHYAEFPYT

SEQ ID NO: 43 - protein, 11 aa, synthetic construct
KASQDIQSYLS

SEQ ID NO: 44 - protein, 7 aa, synthetic construct
RANRLVD

SEQ ID NO: 45 - protein, 9 aa, synthetic construct
LHYAEFPYT

SEQ ID NO: 46 - protein, 11 aa, synthetic construct
KASQDIQSYLS

SEQ ID NO: 47 - protein, 7 aa, synthetic construct
RANRLVD

SEQ ID NO: 48 - protein, 9 aa, synthetic construct
LHYAEFPYT

SEQ ID NO: 49 - protein, 107 aa, synthetic construct
DIQMTQSPSSLSASVGDRVTITCKASQDIQSYLSWFQQKPGKAPKTLIYRANRLVDGVPSRFSGSGSGTDFTLTISSLQPEDFATYYCLHYAEFPYTFGGGTKVEIK

SEQ ID NO: 50 - DNA, 321 bp, synthetic construct
  source 1..321: mol_type = other DNA
gatattcagatgacccagagcccgagcagcctgagcgcgagcgtgggcgatcgcgtgaccattacctgcaaagcgagccaggatattcagagctatctgagctggtttcagcagaaaccgggcaaagcgccgaaaaccctgatttatcgcgcgaaccgcctggtggatggcgtgccgagccgctttagcggcagcggcagcggcaccgattttaccctgaccattagcagcctgcagccggaagattttgcgacctattattgcctgcattatgcggaatttccgtatacctttggcggcggcaccaaagtggaaattaaa

SEQ ID NO: 51 - protein, 214 aa, synthetic construct
DIQMTQSPSSLSASVGDRVTITCKASQDIQSYLSWFQQKPGKAPKTLIYRANRLVDGVPSRFSGSGSGTDFTLTISSLQPEDFATYYCLHYAEFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - DNA, 642 bp, synthetic construct
  source 1..642: mol_type = other DNA
gatattcagatgacccagagcccgagcagcctgagcgcgagcgtgggcgatcgcgtgaccattacctgcaaagcgagccaggatattcagagctatctgagctggtttcagcagaaaccgggcaaagcgccgaaaaccctgatttatcgcgcgaaccgcctggtggatggcgtgccgagccgctttagcggcagcggcagcggcaccgattttaccctgaccattagcagcctgcagccggaagattttgcgacctattattgcctgcattatgcggaatttccgtatacctttggcggcggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgc

SEQ ID NO: 53 - protein, 8 aa, synthetic construct
GFTFSNYW

SEQ ID NO: 54 - protein, 10 aa, synthetic construct
IRLKSNNYAT

SEQ ID NO: 55 - protein, 10 aa, synthetic construct
TPFYGSSFPY

SEQ ID NO: 56 - protein, 5 aa, synthetic construct
NYWMN

SEQ ID NO: 57 - protein, 19 aa, synthetic construct
EIRLKSNNYATHYAESVKG

SEQ ID NO: 58 - protein, 8 aa, synthetic construct
FYGSSFPY

SEQ ID NO: 59 - protein, 10 aa, synthetic construct
GFTFSNYWMN

SEQ ID NO: 60 - protein, 19 aa, synthetic construct
EIRLKSNNYATHYAESVKG

SEQ ID NO: 61 - protein, 8 aa, synthetic construct
FYGSSFPY

SEQ ID NO: 62 - protein, 119 aa, synthetic construct
ELQLVESGGGLVQPGGSLRLSCAASGFTFSNYWMNWVRQAPGKGLEWVAEIRLKSNNYATHYAESVKGRFTISRDNAKNSLYLQMNSLRAEDTAVYYCTPFYGSSFPYWGQGTLVTVSS

SEQ ID NO: 63 - DNA, 357 bp, synthetic construct
  source 1..357: mol_type = other DNA
gaactgcagctggtggaaagcggcggcggcctggtgcagccgggcggcagcctgcgcctgagctgcgcggcgagcggctttacctttagcaactattggatgaactgggtgcgccaggcgccgggcaaaggcctggaatgggtggcggaaattcgcctgaaaagcaacaactatgcgacccattatgcggaaagcgtgaaaggccgctttaccattagccgcgataacgcgaaaaacagcctgtatctgcagatgaacagcctgcgcgcggaagataccgcggtgtattattgcaccccgttttatggcagcagctttccgtattggggccagggcaccctggtgaccgtgagcagc

SEQ ID NO: 64 - protein, 446 aa, synthetic construct
ELQLVESGGGLVQPGGSLRLSCAASGFTFSNYWMNWVRQAPGKGLEWVAEIRLKSNNYATHYAESVKGRFTISRDNAKNSLYLQMNSLRAEDTAVYYCTPFYGSSFPYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 65 - DNA, 1338 bp, synthetic construct
  source 1..1338: mol_type = other DNA
gaactgcagctggtggaaagcggcggcggcctggtgcagccgggcggcagcctgcgcctgagctgcgcggcgagcggctttacctttagcaactattggatgaactgggtgcgccaggcgccgggcaaaggcctggaatgggtggcggaaattcgcctgaaaagcaacaactatgcgacccattatgcggaaagcgtgaaaggccgctttaccattagccgcgataacgcgaaaaacagcctgtatctgcagatgaacagcctgcgcgcggaagataccgcggtgtattattgcaccccgttttatggcagcagctttccgtattggggccagggcaccctggtgaccgtgagcagcgcgagcaccaaaggcccgagcgtgtttccgctggcgccgtgcagccgcagcaccagcgaaagcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcaccaaaacctatacctgcaacgtggatcataaaccgagcaacaccaaagtggataaacgcgtggaaagcaaatatggcccgccgtgcccgccgtgcccggcgccggaatttctgggcggcccgagcgtgtttctgtttccgccgaaaccgaaagataccctgatgattagccgcaccccggaagtgacctgcgtggtggtggatgtgagccaggaagatccggaagtgcagtttaactggtatgtggatggcgtggaagtgcataacgcgaaaaccaaaccgcgcgaagaacagtttaacagcacctatcgcgtggtgagcgtgctgaccgtgctgcatcaggattggctgaacggcaaagaatataaatgcaaagtgagcaacaaaggcctgccgagcagcattgaaaaaaccattagcaaagcgaaaggccagccgcgcgaaccgcaggtgtataccctgccgccgagccaggaagaaatgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagccgcctgaccgtggataaaagccgctggcaggaaggcaacgtgtttagctgcagcgtgatgcatgaagcgctgcataaccattatacccagaaaagcctgagcctgagcctgggcaaa

SEQ ID NO: 66 - protein, 6 aa, synthetic construct
QDISNY

SEQ ID NO: 67 - skipped

SEQ ID NO: 68 - protein, 9 aa, synthetic construct
QQGNALPFT

SEQ ID NO: 69 - protein, 11 aa, synthetic construct
RASQDISNYLN

SEQ ID NO: 70 - protein, 7 aa, synthetic construct
YTSILQL

SEQ ID NO: 71 - protein, 9 aa, synthetic construct
QQGNALPFT

SEQ ID NO: 72 - protein, 11 aa, synthetic construct
RASQDISNYLN

SEQ ID NO: 73 - protein, 7 aa, synthetic construct
YTSILQL

SEQ ID NO: 74 - protein, 9 aa, synthetic construct
QQGNALPFT

SEQ ID NO: 75 - protein, 107 aa, synthetic construct
DIQMTQSPSSLSASVGDRVTITCRASQDISNYLNWYQQKPGKVPKLLIYYTSILQLGVPSRFSGSGSGTDYTLTISSLQPEDVATYYCQQGNALPFTFGGGTKVEIK

SEQ ID NO: 76 - DNA, 321 bp, synthetic construct
  source 1..321: mol_type = other DNA
gatattcagatgacccagagcccgagcagcctgagcgcgagcgtgggcgatcgcgtgaccattacctgccgcgcgagccaggatattagcaactatctgaactggtatcagcagaaaccgggcaaagtgccgaaactgctgatttattataccagcattctgcagctgggcgtgccgagccgctttagcggcagcggcagcggcaccgattataccctgaccattagcagcctgcagccggaagatgtggcgacctattattgccagcagggcaacgcgctgccgtttacctttggcggcggcaccaaagtggaaattaaa

SEQ ID NO: 77 - protein, 214 aa, synthetic construct
DIQMTQSPSSLSASVGDRVTITCRASQDISNYLNWYQQKPGKVPKLLIYYTSILQLGVPSRFSGSGSGTDYTLTISSLQPEDVATYYCQQGNALPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - DNA, 642 bp, synthetic construct
  source 1..642: mol_type = other DNA
gatattcagatgacccagagcccgagcagcctgagcgcgagcgtgggcgatcgcgtgaccattacctgccgcgcgagccaggatattagcaactatctgaactggtatcagcagaaaccgggcaaagtgccgaaactgctgatttattataccagcattctgcagctgggcgtgccgagccgctttagcggcagcggcagcggcaccgattataccctgaccattagcagcctgcagccggaagatgtggcgacctattattgccagcagggcaacgcgctgccgtttacctttggcggcggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgc